(19) United States
(12) Patent Application Publication
(10) Pub. No.: US 2022/0249151 A1
(43) Pub. Date: Aug. 11, 2022

(54) TREATMENT PLANNING AND MONITORING FOR IRE AND H-FIRE PROTOCOLS

(71) Applicant: AngioDynamics, Inc., Latham, NY (US)

(72) Inventors: HAYLEY FORREST, NATICK, MA (US); NAVID MANUCHEHRABADI, MARLBOROUGH, MA (US)

(73) Assignee: AngioDynamics, Inc., LATHAM, NY (US)

(21) Appl. No.: 17/725,283

(22) Filed: Apr. 20, 2022

Related U.S. Application Data
(63) Continuation-in-part of application No. 17/229,632, filed on Apr. 13, 2021.
(60) Provisional application No. 63/177,007, filed on Apr. 20, 2021.
(60) Provisional application No. 63/031,282, filed on May 28, 2020.
(60) Provisional application No. 63/009,040, filed on Apr. 13, 2020.

(51) Int. Cl. A61B 18/12; A61N 1/32

(52) U.S. Cl. CPC A61B 18/1206; A61B 2018/00577; A61N 1/327

(57) ABSTRACT

Provided are techniques to compare current across treatment plans as well as between various clinician and/or clinic protocols. Further provided are devices and methods to generate a recommended treatment plan for an ablation modality like IRE or H-FIRE to treat a tumor. Current from an ablation therapy treatment can be measured and normalized to compare the normalized current between ablation therapy treatments. An efficacy of a treatment, or a completion of a treatment, can be determined based on a change in the normalized current. Also provided is a database of treatment results including indications of normalized current from the treatments.

### CROSS-REFERENCE TO RELATED APPLICATIONS

This application claims priority to U.S. Provisional Patent Application No. 63/177,007, filed on Apr. 20, 2021, and is a continuation-in-part of U.S. patent application Ser. No. 17/229,632, filed on Apr. 13, 2021, which claims priority to U.S. Provisional Application No. 63/009,040, filed on Apr. 13, 2020, and to U.S. U.S. Provisional Application No. 63/031,282, filed on May 28, 2020. All these applications are incorporated herein by referenced in their entirety.

### BACKGROUND

Irreversible electroporation (IRE) and high-frequency IRE (H-FIRE) are techniques to cause permanent death of tissue cells in a body, thereby ablating the tissue. IRE and H-FIRE are facilitated by application of voltage pulses to tissue to create short but strong electrical fields to form permanent nanopores in cell membranes of the tissue. The nanopores disrupt the cellular homeostasis leading to cell death. Cell death due to IRE or H-FIRE results from apoptosis. Whereas cell death due to other thermal or radiation based ablation techniques results from necrosis. IRE and H-FIRE are often used to ablate tumors in regions where precision and conservation of the extracellular matrix, blood flow and nerves are important.

Several techniques are available to determine the extent of tissue ablation. For example, clinicians using thermal ablation techniques (e.g., cryo, laser, microwave, RF, etc.) typically rely on temperature and a period of exposure time to determine an extent of ablation. However, currently there is not a reliable method to elucidate the impact of temperature for prolonged IRE or H-FIRE procedures. Thus, clinicians cannot borrow methods from thermal ablation techniques to design and plan treatment for IRE or H-FIRE procedures.

### BRIEF SUMMARY

The present disclosure provides systems and techniques that determine when and how to apply IRE and/or H-FIRE based on a tumor (e.g., target tissue) immunoscore and/or a tumor type. For example, invasive and/or noninvasive immunomonitoring technologies can be used to determine an immunoscore for a tumor. The tumor can be classified into one of several types or classes based on the immunoscore. As a specific example, the tumor can be classified as one of a cold tumor, an excluded tumor, an immunosuppressed tumor, or a hot tumor. Given the tumor type and optionally, immunogenicity and/or adjuvanticity information about the tumor, a treatment “intensity” can be determined. Further, the present disclosure provides a system and method to determine when the treatment intensity has been met.

In addition to the above, the present disclosure provides to compare current across treatment plans as well as between various clinician and/or clinic protocols. In general, the present disclosure provides to normalize the current response from a treatment and compare normalized current between treatments. Further, the present disclosure provides to determine an efficacy of a treatment, or a completion of a treatment based on a percentage rise in the normalized current. For example, the intensity of treatment can be determined based on the normalized current response. This provides significant advantages over conventional techniques clinicians use to identify the completion of a treatment. Furthermore, the present disclosure overcomes difficulties associated with comparing different treatments, which have prevented sharing of data between clinicians and clinics.

As such, the present disclosure can be implemented by clinics to form databases or “banks” of tissue specific treatments representing treatments from multiple clinicians and even multiple clinics. This database can include information about normalized current from these procedures, which can be utilized by clinicians or researchers for treatment planning. Furthermore, ablation therapy devices could compare normalized current for an in-process therapy procedure to normalized current from the database to determine an efficacy or the in process therapy procedure, or suggest continued treatment procedure parameters (e.g., additional voltage pulses, or the like).

As noted above, tumors can be categorized into cold, immunosuppressed, excluded or hot tumor types based on an immunoscore. The efficacy, dosage, and type of treatment depends on the immunoscore of the tumor. Often, the goal of new immunotherapies for treating tumors focuses on moving the balance from a pro-tumor to an anti-tumor microenvironment to allow the immune system to maximize an efficient anti-tumor response. Changes in the tumor microenvironment depend on the type and the progression of cell death mechanisms over time before and after treatment. Immunogenicity and adjuvanticity are tumor-intrinsic core characteristics that contribute to the shaping of the tumor-associated T cell landscape. The present disclosure postulates that tumor-associated T cells are strikingly low or absent in cold tumors, although they may contribute to a certain extent to other subtypes.

As such, the present disclosure provides systems and techniques to generate a therapeutic scheme, or said differently, to narrow down the list of possible IRE and/or H-FIRE treatment therapies by excluding potentially inefficient combinations based on the classification of a tumor into one of several categories. For example, the present disclosure provides a system configured to receive an indication of a type of tumor being targeted. As a specific example, the system can be configured to receive an indication that the type of tumor being targeted is one of a cold tumor, an immunosuppressed tumor, an excluded tumor, or a hot tumor. The system can further be configured to receive an indication of an immunogenicity or an adjuvanticity of the tumor. Responsive to receiving the indication of the type of tumor and/or characteristics of the tumor, the system can generate and display a recommended treatment intensity or treatment zone for an IRE and/or H-FIRE procedure aimed at achieving a desired innate and/or adaptive immune response.

The present disclosure can be implemented in conjunction with additional adjunctive or supporting immune treatment therapies, such as, drugs or other treatments. The use of such conjunctive supporting immune treatment therapies may be part of an overall treatment plan. For example, a system configured according to the present disclosure can generate both a recommended IRE and/or H-FIRE intensity and a recommended conjunctive supporting immune treatment therapy based on the type of tumor being treated. For example, the system can be arranged to recommend an ablation modality (e.g., IRE, H-FIRE, or the like) for an ablation therapy, an intensity of the ablation therapy, and a time to implement the ablation therapy with respect to additional immune therapies.

A benefit to the present disclosure is that the complexity of IRE and/or H-FIRE ablation volume can be simplified into “intensity” zones, or said differently, the IRE and/or H-FIRE protocol can be characterized to induce different types of intensity. In some examples, the intensity is categorized into three zones, such as, zone 1, zone 2, and zone 3. In such an example, zone 1 can correspond to the lowest or least intensity while zone 3 can correspond to the highest or greatest intensity or IRE or H-FIRE. The different IRE or H-FIRE intensities corresponding to the different zones affect the tumor microenvironment with different cell death mechanisms.

A further benefit to the present disclosure, or said differently, to representing current using equations given herein and normalizing the current to compare current across treatments is that the present disclosure can be used to simplify the impact of the numerous variables associated with ablation volume in IRE and H-FIRE treatments. A number of additional benefits can be realized from the present disclosure such as, for example: predicting the current response after an increase or decrease in the applied voltage before or during a procedure; avoiding overcurrent, especially at higher voltages, by using typical normalized current increases over a certain number of pulses; accessing and compare data between clinicians or clinics; generating a bank of results for various tissue types to compare treatments with; directly comparing current response between voltages by removing the offsets to better predict the effectiveness of a specific protocol; predicting a current phase of treatment given the normalized current and a derived rate of change; and estimating the dynamic electrical conductivity of tissue.

These and other examples are described in greater detail below. In the following description, numerous specific details such as processor and system configurations are set forth in order to provide a more thorough understanding of the described embodiments. However, the described embodiments may be practiced without such specific details. Additionally, some well-known structures (e.g., circuits, specific treatment protocols, and the like) have not been shown in detail, to avoid unnecessarily obscuring the described embodiments.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1A and FIG. 1B illustrate an ablation therapy system in accordance with at least one embodiment.

FIG. 2A illustrates a graph in accordance with at least one embodiment.

FIG. 2B illustrates another graph in accordance with at least one embodiment.

FIG. 3A illustrates another graph in accordance with at least one embodiment.

FIG. 3B illustrates another graph in accordance with at least one embodiment.

FIG. 4 illustrates feedback system in accordance with at least one embodiment.

FIG. 5A and FIG. 5B illustrate probe pairs in accordance with at least one embodiment.

FIG. 6A and FIG. 6B illustrates a plot of voltage pulses in accordance with at least one embodiment.

FIG. 7 illustrates an ablation therapy device in accordance with at least one embodiment.

FIG. 8 illustrates a routine in accordance with at least one embodiment.

FIG. 9 illustrates another routine in accordance with at least one embodiment.

FIG. 10A illustrates a plot in accordance with at least one embodiment.

FIG. 10B illustrates another plot in accordance with at least one embodiment.

FIG. 11 illustrates another plot in accordance with at least one embodiment.

FIG. 12 illustrates another plot in accordance with at least one embodiment.

FIG. 13 illustrates another routine in accordance with at least one embodiment.

FIG. 14 illustrates a graphical user interface (GUI) in accordance with at least one embodiment.

FIG. 15 illustrates another routine in accordance with at least one embodiment.

FIG. 16 illustrates another routine in accordance with at least one embodiment.

FIG. 17 illustrates another routine in accordance with at least one embodiment.

FIG. 18A illustrates another plot in accordance with at least one embodiment.

FIG. 18B illustrates another plot in accordance with at least one embodiment.

FIG. 19 illustrates another plot in accordance with at least one embodiment.

FIG. 20 illustrates diagram in accordance with at least one embodiment.

FIG. 21A illustrates a flow diagram in accordance with at least one embodiment.

FIG. 21B illustrates another aspect of the subject matter in accordance with one embodiment.

FIG. 22 illustrates another GUI in accordance with at least one embodiment.

FIG. 23 illustrates a flow diagram in accordance with at least one embodiment.

FIG. 24 illustrates another diagram in accordance with at least one embodiment.

FIG. 25 illustrates another diagram in accordance with at least one embodiment.

FIG. 26 illustrates another diagram in accordance with at least one embodiment.

FIG. 27 illustrates another diagram in accordance with at least one embodiment.

FIG. 28 illustrates another diagram in accordance with at least one embodiment.

FIG. 29 illustrates another diagram in accordance with at least one embodiment.

FIG. 30 illustrates another diagram in accordance with at least one embodiment.

FIG. 31 illustrates another diagram in accordance with at least one embodiment.

FIG. 32 illustrates an ablation therapy system in accordance with at least one embodiment.

FIG. 33 illustrates a machine learning training environment in accordance with at least one embodiment.

FIG. 34 illustrates a technique in accordance with at least one embodiment.

FIG. 35 illustrates a computer-readable storage medium in accordance with at least one embodiment.

FIG. 36A illustrates a graphical user interface in accordance with at least one embodiment.

FIG. 36B illustrates another graphical user interface in accordance with at least one embodiment.

FIG. 36C illustrates another graphical user interface in accordance with at least one embodiment.

FIG. 36D illustrates another graphical user interface in accordance with at least one embodiment.

FIG. 37A illustrates another graphical user interface in accordance with at least one embodiment.

FIG. 37B illustrates another graphical user interface in accordance with at least one embodiment.

FIG. 38A illustrates a plot for a graphical user interface in accordance with at least one embodiment.

FIG. 38B illustrates another plot for a graphical user interface in accordance with at least one embodiment.

FIG. 39 illustrates a machine in the form of a computer system in accordance with at least one embodiment.

FIG. 40A illustrates a graphical information element in accordance with at least one embodiment.

FIG. 40B illustrates another graphical information element in accordance with at least one embodiment.

FIG. 40C illustrates another graphical information element in accordance with at least one embodiment.

### DETAILED DESCRIPTION

As noted, IRE and H-FIRE are tissue ablation techniques where voltage pulses are applied to tissue to create an electric field and cause cell death. Such treatments are often used to treat cancerous tissue. Cancerous tissue or cancer cells comprise six biological capabilities acquired during the multistep development of human tumors. These “hallmarks” of cancer constitute an organizing principle for rationalizing the complexities of neoplastic disease. They include sustaining proliferative signaling, evading growth suppressors, resisting cell death, enabling replicative immortality, inducing angiogenesis, and activating invasion and metastasis. Underlying these hallmarks are genome instability, which generates the genetic diversity that expedites their acquisition, and inflammation, which fosters multiple hallmark functions. Conceptual progress in the last decade has added two emerging hallmarks of potential generality to this list, reprogramming of energy metabolism and evading immune destruction. In addition to cancer cells, tumors exhibit another dimension of complexity in that they contain a repertoire of recruited, ostensibly normal cells that contribute to the acquisition of hallmark traits by creating the “tumor microenvironment.” The present disclosure provides treatments aimed at changing this microenvironment to increase an immune response in the tissue being treated. The present disclosure classified tissue to be treated, and tumors to be treated into one of several classes.

For example, the tumors can be classified into cold tumors, excluded tumors, immunosuppressed tumors, and hot tumors. Each of these types of tumors have respective unique characteristics. For example, hot tumors often have: a high degree of T cell and cytotoxic T cell infiltration (resulting in a high immunoscore); checkpoint activation (e.g., programmed cell death protein 1 (PD-1), cytotoxic T lymphocyte-associated antigen 4 (CTLA4), T cell immunoglobulin mucin receptor 3 (TIM3) and lymphocyte activation gene 3 (LAG3)) or otherwise impaired T cell functions (e.g., extracellular potassium-driven T cell suppression). As another example, tumors classified as immunosuppressed tumors often have: poor, albeit not absent, T cell and cytotoxic T cell infiltration (resulting in an intermediate immunoscore); the presence of soluble inhibitory mediators (e.g., transforming growth factor-β (TGFβ), interleukin 10 (IL-10) and vascular endothelial growth factor (VEGF)); the presence of immune suppressive cells (e.g., myeloid-derived suppressor cells and regulatory T cells); the presence of T cell checkpoints (PD-1, CTLA4, TIM3 and LAG3). With yet another example, tumors classified as excluded tumors often have: no T cell infiltration inside the tumor borders (e.g., an invasive margin) resulting in an intermediate immunoscore; activation of oncogenic pathways; epigenetic regulation and reprogramming of the tumor microenvironment; aberrant tumor vasculature and/or stroma; and hypoxia. In a further example, tumors classified as cold tumors often have: an absence of T cells within the tumor and at the tumor edges (resulting in a low immunoscore); failed T cell priming (e.g., low tumor mutational burden, poor antigen presentation and intrinsic insensitivity to T cell killing).

These different types of tumors can be determined, for example, based on an immunoscore of the tissue. For example, illustrative examples of these different types of tumors are known in the art and can be generated, for example, using various cell staining technologies. Such illustrative examples depict the level and spatial distribution of CD3 and CD8T cell infiltration differences between distinct solid tumor phenotypes (e.g., hot (or inflamed); altered, which can be excluded or immunosuppressed; and cold (or non-inflamed). These tumor phenotypes are characterized by high, intermediate and low immunoscores, respectively.

As indicated, the present disclosure provides systems and methods to generate a treatment plan to provoke immunomodulation of the tumor microenvironment to induce an anti-tumor immune response. In situ vaccines result in intratumoral modulation to attract and activate dendritic cells able to present the full antigenic repertoire to tumor-specific T cells able to kill tumor cells. This immunomodulation can occur at different levels or with different modalities of treatment. For example, one such modality can be stimulating the induction of immunogenic cell death with radiotherapy, electrochemotherapy, hyperthermia, photodynamic therapy or oncolytic viruses. Another such modality can be increasing the number and maturation of dendritic cells through the administration of growth factors, cytokines or TLR agonists. Another modality can be to stimulate the priming and activation of T cells through the intratumoral injection of checkpoint inhibitors, cytokines or other immunomodulating agents. In another modality, the direct killing of cancer cells can be promoted through the local administration of STING agonists or checkpoint inhibitors. All of these modalities can be combined in order to induce a robust anti-tumor immune response.

The cytokines/chemokines and enzymes secreted by cancer-associated fibroblasts. The cancer-associated fibroblast-secreted or exported cytokines, chemokines, inhibitory and ECM remodeling molecules together regulate immune cell function. Inhibitory molecules mainly include PD-L1, PD-L2, FASL, TGF-β and kynurenine (Kyn). Cytokines and chemokines include M-CSF1, VEGF, TNF, PGE2, IL-13/26, IL-1β, IL-5/6, IL-10, CXCL1-2, CXCL5-6, CXCL8-10, CXCL12, CCL2, CCL2-3, CCL5, etc. Representative ECM remodeling molecules are matrix metalloproteinase (MMPs), Chi3L1, and hyaluronic acid. Together, these molecules secreted by CAFs suppress NK cell and cytotoxic T cell activity.

The present disclosure provides an ablation therapy system and methods of ablation therapy configured to generate a recommendation and/or treatment protocol with the intent to immunomodulate the tumor microenvironment to induce an anti-tumor immune responses. For example, such a system can recommend a “treatment intensity” or a “treatment zone” with which to treat the tumor.

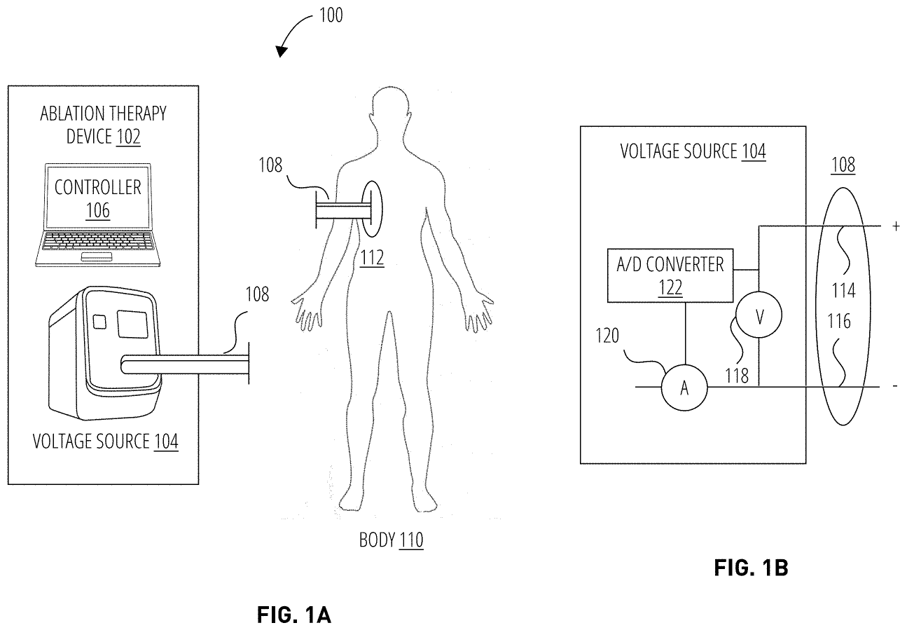

FIG. 1A illustrates an ablation therapy system 100. Ablation therapy system 100 can be the NanoKnife® system available from AngioDynamics®. Ablation therapy system 100 includes an ablation therapy device 102 with which a clinician can apply an ablation therapy procedure to a body 110 of a patient. For example, a clinician can use the ablation therapy device 102 to apply an IRE or H-FIRE ablation therapy to body 110. Ablation therapy device 102 comprises a voltage source 104, a controller 106, and at least one probe 108. The voltage source 104 may comprise a generator configured to generate electrical pulses. The probe 108 may include, but is not limited to, a single monopolar probe with a single electrode (for example, up to 6 monopolar electrode probes may be used for a single IRE and/or H-FIRE treatment); a single bipolar probe comprising at least two electrodes; a single probe with multiple tines and each tine having either a single monopolar electrode or at least one tine having at least two electrodes; and/or a surface electrode with a single electrode. Table 1 below provides non-limiting examples of the various probe 108 an electrode configurations that may be used with the system

TABLE 1

| | |
|---|---|
| Electrode | Parallel plate; 0.1 millimeter (mm) to 70 mm diameter (and larger |
| Type | for applications relating to e.g., whole organ decellularization); |
| | Needle electrode(s): 0.001 mm to 1 centimeter (cm) diameter; |
| | Single probe with embedded disk electrodes: 0.001 mm to 1 cm |
| | diameter; Spherical electrodes: 0.0001 mm to 1 cm diameter; Needle |
| | diameter: 0.001 mm to 1 cm; Electrode length (needle): 0.1 mm to |
| | 30 cm; Electrode separation: 0.1 mm to 5 cm, or even 5 cm to 20 |
| | cm, or 20 cm to 100 cm, and larger (for reversible electroporation, |
| | gene delivery, or positive electrode with ground patch on patient's |
| | exterior.) |
| Probe | Single monopolar probe with a single needle electrode; Multiple |
| Type | monopolar probes (e.g., 2, 4, 6, or more) with a single needed |
| | electrode; Single bipolar probe with a bipolar needle electrode; |
| | Multiple bipolar probes (e.g., 2, 4, 6, or more) with a bipolar needle |
| | electrode; Single catheter probe with multiple electrodes; Single |
| | probe with multiple monopolar electrode tines; A number of surface |
| | electrodes (e.g., 2, 4, 6, or more) with either a single surface |
| | electrode, multiple surface electrodes, or bipolar surface |
| | electrode(s); Catheter comprising a circular loop and/or ring |
| | comprising a series of electrodes; Catheter comprising an balloon |
| | with electrodes placed on a balloon outer surface; or A catheter |
| | comprising an expandable flower shaped distal end with a series of |
| | electrodes |

In general, a clinician can use the ablation therapy device 102 to apply a series of voltage pulses to target tissue 112 of body 110 via the probe 108. The present disclosure can be applied to any type of target tissue 112, such as, for example, liver, prostate, kidney, pancreas, lung, head, neck, heart, brain, or other soft tissue areas of body 110. Although the present disclosure does not attempt to provide exhaustive examples of different ablation therapy protocols, a general ablation therapy is described. In general, an ablation therapy for which the present disclosure can be implemented will include application of a series of voltage pulses to target tissue 112. For example, voltage source 104 can repeatedly energize probe 108 to deliver a series of voltage pulses to target tissue 112.

In one aspect, at least a portion of the probe 108 can be configured for insertion into target tissue 112 of body 110 of a patient. Probe 108 can be any type including but not limited to the probes described in Table 1 above. Although not depicted, probe 108 can comprise a handle, a needle having a proximal end and a distal end, and at least one connector (e.g., to couple to voltage source 104). In some examples, the needle can comprise at least one electrode and a tip positioned at the distal end of the needle. The tip can be a sharp tip capable of piercing tissue of body 110. As used herein, probe 108 can include any number of pairs of probes. Alternatively, ablation therapy device 102 could include multiple probe 108 with which to form probe pairs. The term “probe pair” includes electrodes coupled to voltage source 104 and arranged to deliver a voltage to target tissue 112. It is noted that probe and electrode are often used interchangeably in this disclosure. Further, probe 108 can include multiple probe pairs. For example, probe 108 could include four probes which combined can be used to form 6 different pairs of probes.

As outlined above, IRE is a tissue ablation technique where high voltage electrical pulses are applied to target tissue in a target area or a treatment site. Included in IRE is H-FIRE, which includes short electrical pulses commonly having a biphasic waveform, as described in more detail below. Consequently, current circulation through the target tissue 112 can create an electrical field based on the spatial distribution of electrical properties of tissue that ultimately triggers different cell death mechanisms. In general, an IRE and/or H-FIRE protocols involve delivering a series of short and intense electric pulses through electrodes inserted directly into, or around, target tissue 112 and/or on the surface of a patient's body 110. The pulses are designed to generate an electric field, between the electrodes, capable of inducing a rapid buildup of charge across the plasma membrane of cells. The charge across the plasma membrane of a cell is commonly referred to as a transmembrane potential (TMP).

Once the TMP reaches a critical voltage, it is thought that electrically conductive pores form in the membrane to prevent permanent damage by shunting current and limiting further TMP rise. If the pulse amplitude and duration are tuned to permit pore resealing, and cell viability is maintained following exposure, the process is categorized as reversible electroporation. However, where pore resealing does not take place, cell death occurs, and the process is categorized as IRE.

As known in the art, a target tissue (e.g., target tissue 112) has been successfully irreversibly electroporated when tissue cells are unable to seal the pores formed in the plasma membrane of the cells. A threshold voltage gradient (v/cm) and threshold number of pulses are required to achieve irreversible electroporation.

It is within the conception of this disclosure to provide a user with a system and method to both determine an intensity with which to apply these parameters and also to generate a recommended set of these parameters. For example, the controller 106 can be arranged to receive (e.g., via a graphical user interface, via input devices, etc.) an indication of the type of tumor being targeted (e.g., including, but not limited to, hot tumor, cold tumor, immunosuppressed tumor, and/or excluded tumor). Furthermore, the controller 106 can be arranged to receive an indication of an immunoscore of a patient. Given the tumor type and/or immunoscore, the controller 106 can generate a recommended treatment protocol. In some examples, the treatment protocol can be associated with a treatment zone (described in greater detail below) and can be generated with the intent to ablate the target tumor and achieve a maximum innate and/or adaptive immune response. With some examples, the controller 106 can recommend an overall therapy protocol including additional adjunctive or supporting immune treatment therapies (e.g., drugs or other treatments). The use of such conjunctive supporting immune treatment therapies may be part of the overall treatment plan. For example, the controller 106 may generate a recommended conjunctive supporting immune treatment therapy based on the type of tumor being treatment and/or the patient's immunoscore as well as a respective timeline of when each therapy should be administered.

As the present disclosure provides to generate a recommended treatment intensity or treatment zone based on a tumor type or immunoscore, a detailed description of treatment zones and intensities is provided. The treatment zone or intensity is characterized by a normalized current measurement. Said differently, the normalized current measurement provides clinicians with an indication of what ablation modality the target tissue is undergoing at a particular point in the treatment as well as transitions between ablation modalities.

Electrical conductivity of the target tissue 112 will rise upon the delivery of treatment pulses. The measurement of electrical conductivity is a known indicator for determining the extent of electroporation in tissue and can be used to determine if cells of the target tissue 112 have been successfully irreversibly electroporated. However, direct measurement of the electrical conductivity of a target tissue is not a practical clinical approach as it highly depends not only on the shape factor (extrinsic property) but also on many other intrinsic properties which might vary during an IRE and/or H-FIRE procedure. This makes direct measurements of electrical conductivity, if not impractical, very difficult to be implemented and used. The electrical conductivity measurements for H-FIRE could be more complicated as it is frequency dependent. However, as will be described below in more detail, the normalized current may be analyzed during an IRE and/or H-FIRE treatment to determine an IRE and/or H-FIRE treatment endpoint.

H-FIRE protocols are comprised of bipolar and/or biphasic electrical pulses delivered at a higher repetition rate, as opposed to traditional IRE that uses unipolar and/or monopolar electrical pulses delivered at a lower repetition rate as compared to H-FIRE. Biphasic bursts of an H-FIRE protocol with inter-pulse delays can be used to ablate tissue while minimizing the need for a paralytic to avoid muscle contractions often seen in traditional IRE treatments. Furthermore, high-frequency fields have the potential to overcome impedance barriers posed by low conductivity tissues, which could result in more homogenous and predictable treatment outcomes in heterogeneous systems.

H-FIRE also has potential advantages for use in neurosurgery, including the ability to deliver pulses without inducing muscle contraction, inherent selectivity against malignant cells, and the capability of simultaneously opening the blood-brain barrier surrounding regions of ablation. For example, ablation therapy system 100, which includes voltage source 104 is capable of generating electrical pulse parameters capable of being used in either an IRE and/or H-FIRE treatment to deliver the intensity appropriate for the identified tumor type. Pulse parameters and other treatment parameters can be set as standardized parameters for typical IRE and/or H-FIRE treatments, specifically chosen by an end user prior to treatment, or may be changed by a treatment control module of the system during the treatment.

FIG. 1B illustrates a portion of ablation therapy system 100 shown in FIG. 1A. As noted, ablation therapy system 100 is provided to measure current during an IRE or H-FIRE procedure and to normalize the current (among other examples). As illustrated in FIG. 1B, ablation therapy system 100 includes a probe 108 having a positive electrode 114 and a negative electrode 116. Furthermore, ablation therapy system 100, and particularly, voltage source 104 includes a voltage sensor 118 and a current sensor, or ammeter 120. In general, the voltage sensor 118 can be connected across the positive electrode 114 and the negative electrode 116 (e.g., in parallel with the target tissue 112, or the like) while the ammeter 120 can be connected in series with one of the electrodes (e.g., the negative electrode 116 in this case).

Voltage source 104 can further include an analog to digital (A/D) converter 122 coupled to the voltage sensor 118 and the ammeter 120. The A/D converter 122 can further be coupled to controller 106. Sensed values may be periodically, repeatedly, or continuously received and digitized by the A/D converter 122 and transmitted to controller 106. In some examples, the A/D converter 122 can sample the sensed values at a rate of greater than 100 MHz.

With some examples, voltage sensor 118 can be a voltage divider, such as, comprised of two serially connected resistors, which measures a voltage drop across a known resistance value. The voltage sensor 118 can use resistors that are of much higher resistance than the tissue. As a specific example, the resistors in voltage sensor 118 can be in the kilo-ohm (ku) to mega-ohm (Me) range whereas target tissue 112 may typically have a resistance in the hundreds of ohms (a). As such, the voltage sensor 118 may have a negligible voltage drop relative to the target tissue 112.

In some examples, the ammeter 120 can be a Hall effect sensor positioned around an electrode so as to measure electric current without directly interfering with the voltage pulse. Typically, the ammeter 120 is placed on the negative side (e.g., negative electrode 116) of the pair of electrodes in probe 108. Where more than two electrodes are present, multiple ammeters 120 can be provided.

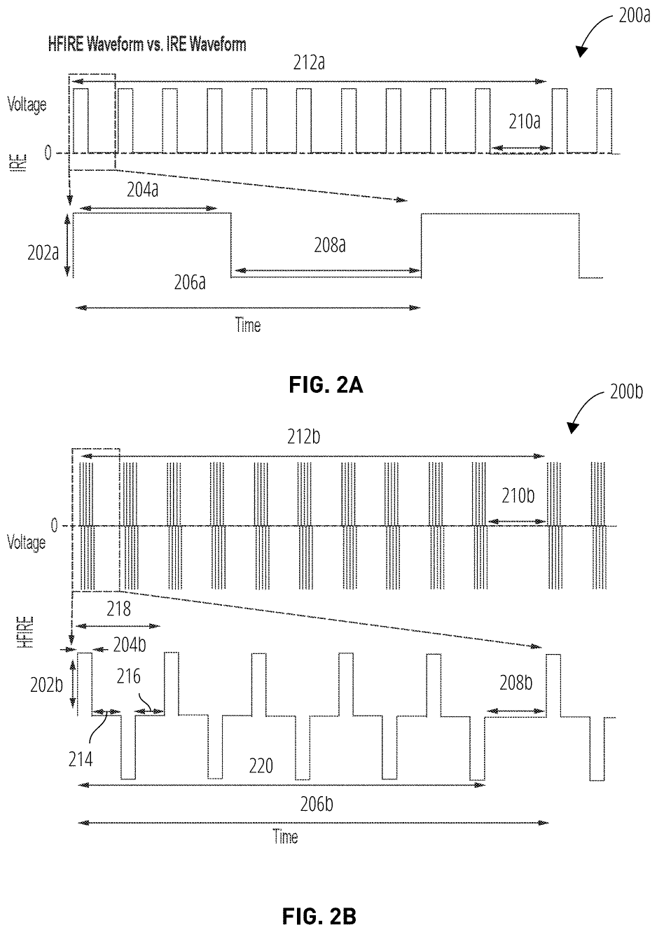

FIG. 2A shows graph 200a while FIG. 2B shows graph 200b. Graphs 200a and 200b depict IRE and HFIRE waveforms, respectively. As shown in graph 200a, a typical IRE waveform comprises a pulse amplitude 202a, a pulse width 204a, a pulse interval 206a, a delay between pulses 208a, a delay between pulse trains 210a, and a pulse time 212a (i.e., the time of one pulse train).

Graph 200b shows a typical H-FIRE waveform that comprises a pulse amplitude 202b, a pulse width 204b, a pulse interval 206b, an intraphase delay 214, an interpulse delay 216, a bipolar pulse period 218, a pulse width 220, a delay between pulses 208b, a delay between pulse trains 210b, and a pulse time 212b. The on time (i.e., the total time of energy delivery per burst) for H-FIRE is typically equivalent to the length of one IRE pulse (e.g., 100 microseconds (μB)). The bursts per minute for H-FIRE are equivalent to pulses per minute (PPM) for IRE (e.g., 90 PPM, or the like). The train is a set of pulses (or bursts), referred to as pulses delivered consecutively before an extended delay.

Table 2 provides a list of electric pulse parameters that can be generated by the voltage source 104 and manipulated during treatment procedures discussed herein to achieve reversible electroporation (RE) ablation, IRE ablation, H-FIRE ablation, or electrolysis within a treatment site.

TABLE 2

| Mechanism | RE | | IRE | | H-FIRE | | Electrolysis | |
|---|---|---|---|---|---|---|---|---|
| Pulsing polarity | Mono/Biphasic | | Monophasic | | Biphasic | | Mono/Biphasic | |
| Number of pulses | 1-50,000 | pulses | 1-50,000 | pulses | 1-50,000 | pulses | 1-50,000 | pulses |
| delivered | | | | | | | | |
| Electric field | <1,000 | V/cm | 700-5,000 | V/cm | 700-5,000 | V/cm | 1,000-100,000 | V/cm |
| density | | | | | | | | |
| Frequency of pulse | 0.001-1,000 | Hz | 0.001-1,000 | Hz | 0.001-1,000 | Hz | 0.001-1,000 | Hz |
| application | | | | | | | | |
| Frequency of pulse | 0-100 | MHz | 0-100 | MHz | 0-100 | MHz | 0-100 | MHz |
| signal | | | | | | | | |

| | | | | |
|---|---|---|---|---|
| Pulse shape/profile | Square, Triangular, | Square, Triangular, | Square, Triangular, | Square, Triangular, |
| | Trapezoidal, | Trapezoidal, | Trapezoidal, | Trapezoidal, |
| | Exponential | Exponential | Exponential | Exponential |
| | decay, Sawtooth, | decay, Sawtooth, | decay, Sawtooth, | decay, Sawtooth, |
| | Sinusoidal, Alternating | Sinusoidal, Alternating | Sinusoidal, Alternating | Sinusoidal, Alternating |
| | polarity. | polarity. | polarity. | polarity. |
| Pulse type | Positive, Negative, | Positive, Negative, | Positive, Negative, | Positive, Negative, |
| | Neutral electrode | Neutral electrode | Neutral electrode | Neutral electrode |
| | charge pulses (changing | charge pulses (changing | charge pulses (changing | charge pulses (changing |
| | polarity within pulse), | polarity within pulse), | polarity within pulse), | polarity within pulse), |
| | Multiple sets of pulse | Multiple sets of pulse | Multiple sets of pulse | Multiple sets of pulse |
| | parameters for a | parameters for a | parameters for a | parameters for a |
| | single treatment | single treatment | single treatment | single treatment |
| | (changing any of the | (changing any of the | (changing any of the | (changing any of the |
| | above parameters within | above parameters within | above parameters within | above parameters within |
| | the same treatment to | the same treatment to | the same treatment to | the same treatment to |
| | specialize outcome). | specialize outcome). | specialize outcome). | specialize outcome). |
| Voltage (or pulse | 1 Volt (V)-3 kV | 1 kV-10 kV | 1 kV-10 kV | 1 kV-100 kV |
| amplitude) applied | | | | |
| Pulse width | 1 microsecond (μs)-100 μs | 10 μs-100 μs | 1 μs-20 μs | 1 μs-1 minute |
| Intraphase delay | 1 μs-10 μs | N/A | 1-10 μsec | 1-10 μsec |
| Interpulse delay | 1 μs-1,000 μs | N/A | 1-1,000 μsec | 1-1,000 μsec |
| Delay between | 1 μs-1,000 seconds (sec) | 1 μs-1,000 sec | 1 μs-1,000 sec | 1 μs-1,000 sec |
| pulse trains | | | | |

Some clinicians use a threshold change in current measurements during a treatment and/or after a treatment has completed to indicate a desired level of conductivity of the target tissue has been achieved, the impact of IRE or H-FIRE on the target tissue, and/or use a rise in current to indicate an end of treatment. However, intrinsic properties and extrinsic properties can affect the endpoint of treatment directly or indirectly. For example, tissue specific properties (intrinsic property) can affect the current response and therefore the end of treatment. As another example, the probe exposure (extrinsic property) has great impact on the tissue ablation volume and the current trend. For clarity, the current measurements used by clinicians currently known in the art are not the normalized current measurement as described herein.

Current can be directly measured by commercial devices and current is a direct output measurement of the tissue being treated. Moreover, measuring current intrinsically includes all of the unique tissue characteristics as well as unique system input parameters (voltage, pulse trains, etc.) for each treatment. It is also known that electrical conductivity of tissue in a treatment site is a useful parameter to be studied before and after IRE and/or H-FIRE treatment in order to determine a treatment endpoint.

It is known in the art to look for a target rise in the measured current during an IRE and/or H-FIRE procedure to indicate complete irreversible electroporation of the tissue within the target site and an end of treatment. However, it should be noted that in a multi-probe configuration, the last 2-3 probe pairs may not achieve this target amp rise in current because of treatment overlap, and effective electroporation has already occurred in tissue proximate to the last probes. Another factor complicating methods of analyzing IRE and H-FIRE treatment protocols is that output current is voltage dependent. Therefore, increasing voltage for a probe pair might result in the desired target rise in current but that is due to the ohmic effect and not due to electroporation. As such, the observed rise in current may not translate to actual ablation volume or treatment efficacy.

Using conductivity changes as an indication of the extent of electroporation during a treatment may be problematic due to the intrinsic and extrinsic factors unique to a particular procedure. Specifically, voltage and the shape factor impact dynamic conductivity measurements. Equation 1 detailed below illustrates the relationship between normalized current and normalized conductivity, where V=voltage, I=current, S=shape factor, CJ=conductivity, and subscript o denotes initial value.

$$\frac{I}{{I}_{o}} = \frac{S \times σ \times V}{{S}_{o} \times {σ}_{o} \times {V}_{o}} = \frac{σ}{{σ}_{o}} \qquad EQUATION 1$$

Thus, when the shape factor (S) and voltage (V) are held constant during a procedure, normalized current and normalized conductivity will be equal.

Voltage source 104 can, in some examples, be arranged to generate a voltage potential of up to 10,000 Volts. It is within the scope of this disclosure that the voltage source 104 is capable of achieving the various ranges of pulse parameters and/or probe embodiments described in Table 2 above. As way of a non-limiting example, voltage source 104 can be arranged to deliver the voltage potential as a series of pulses where each pulse can have a pulse width of up to 100 μsec. Furthermore, a delay or dwell between voltage pulse up to 2,000 ms (actual delay may depend on cardiac synchronization and/or patient pulse rate) and bursts on time can be up to 200 microseconds. Voltage source 104 can be powered by an A/C power source, D/C power source including a battery. The battery can be rechargeable. For example, voltage source 104 can include an A/C power source arranged to power voltage source 104 where access to A/C power (e.g., 110V, 240V, or the like) is available and to charge the battery such that the battery can power the voltage source 104 where access to A/C power is not available.

Controller 106 can be any of a variety of computing devices coupled to voltage source 104. A clinician can configure the ablation therapy device 102 for a particular ablation therapy protocol. For example, controller 106 can receive input from a clinician associated with the number of probes, the probe pair sequence, the desired voltage, the desired number of pulses, or the like and can send control signals to the voltage source 104 to cause the voltage source 104 to apply voltage pulses the target tissue 112 via probe 108. By way of a non-limiting example, U.S. Publication No. US 2016/0354142, filed Aug. 17, 2016 describes a controller system to be used in combination with the systems, devices, and methods described herein and is incorporated herein by reference.

Additionally, controller 106 can receive indications of current produced by application of the voltage pulses to the target tissue 112 by probe 108. Controller 106 can normalize the current and send control signals to the voltage source 104 based on the normalized current. This and other examples are described more fully herein.

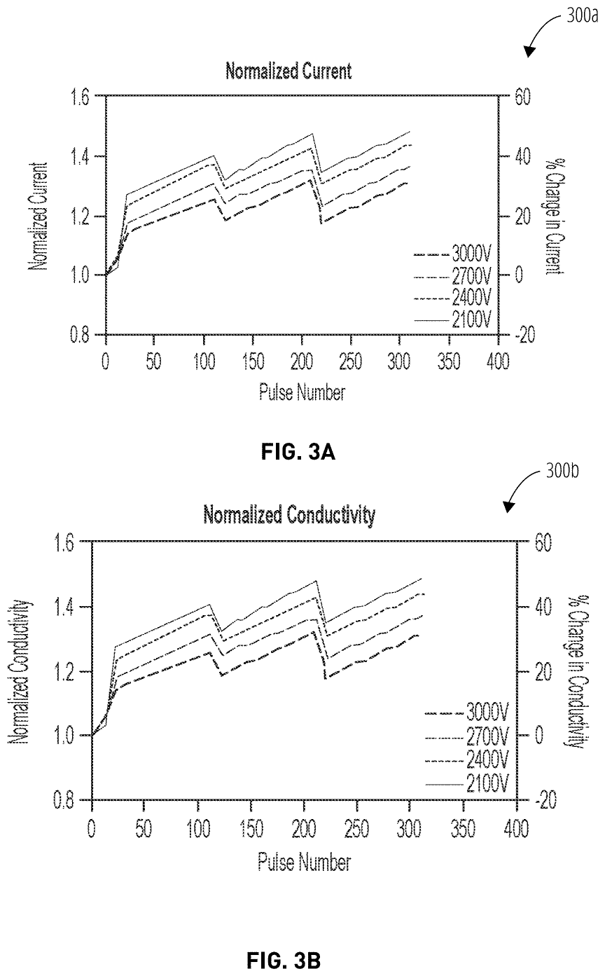

FIG. 3A and FIG. 3B illustrate the relationship between conductivity and current changes for a single procedure. In particular FIG. 3A shows a plot 300a depicting changes in the normalized current while FIG. 3B shows a plot 300b depicting changes in normalized conductivity. Voltage pulses are depicted on the X axis, normalized values on left Y axis and percent change on the right Y axis. Separate trend lines are shown for 3900 V, 2400 V, 2700 V, and 3000 V. The trend lines and individual data points of FIG. 3A and FIG. 3B are identical, illustrating that only when voltage and the shape factor remain constant during a procedure is conductivity and current the same and an accurate indication of tissue changes due to electrical pulses.

Because the shape factor value may vary during the IRE and/or H-FIRE onset (e.g., changes made during a procedure such as probe position, deactivation of a probe pair, and/or modification to exposure length), analysis of the conductivity trends for procedure planning and/or monitoring is not as reliable as an analysis of current trends. This is due to the fact that to be able to estimate the conductivity a set of assumptions are needed, which might not be valid before, during and after the IRE and/or H-FIRE procedure.

An advantage of using the normalized current for treatment planning purposes, as described in more detail herein, is that current inherits the characteristics of extrinsic factors and/or intrinsic factors for individual treatment procedures. For example, as described in more detail below, extrinsic factors and intrinsic factors comprise different patient specific and/or treatment specific parameters. Each individual ablation procedure will comprise a unique set of extrinsic factors and intrinsic factors. These unique extrinsic factors and intrinsic factors will be accounted for as inherent features of measuring the current and then normalizing the current (as described herein). Therefore, each unique extrinsic factor and/or intrinsic factor for an individual treatment procedure will be inherit in the normalization of the current for treatment planning purposes.

Accordingly, this disclosure satisfies a need in the art to create a system and reliable method to determine the efficacy of ablation from IRE and/or H-FIRE and consequently determine a completion to the treatment protocol.

Furthermore, given the number of parameters of an IRE or H-FIRE treatment protocol (e.g., voltage level, number of pulses, pulse polarity, pulse length, delay between pulses, or any of the various pulse parameters in Table 2) it is difficult to compare current output from one treatment to another. These hurdles make comparing results of different treatment protocols difficult as the individual characteristics of the patient coupled with the numerous parameters in an IRE or H-FIRE treatment protocol preclude simply comparing results based on the current measured during the treatment. This difficulty extends to comparing IRE or H-FIRE treatments across clinicians or across treatment centers. These difficulties create problems and inefficiencies in running pilot studies or sharing treatment results between clinicians or clinics. Therefore, a need in the art exists to simplify and accurately compare current output data across various treatments, different treatment protocols, and across different clinicians and/or treatment centers.

For example, the current between two probes may vary based on a number of factors (e.g., voltage, number of pulses, pulse length, delay between pulses, or any of the various pulse parameters in Table 2). Complicating this, current depends on various intrinsic properties of the individual patients (blood perfusion and the extent of vascular structures, thermal properties of tissue, electrical properties of tissue) and extrinsic properties of each treatment (pulsing parameters (IRE or H-FIRE, shape factor, probe placement parameters, etc.).

As will be discussed in more detail below, collecting, analyzing and/or comparing normalized current data during an IRE and/or H-FIRE treatment can be used to provide an end user sufficient information to determine when the cumulative electrical pulses sufficiently result in irreversible electroporation of the tissue within the treatment site. Therefore, normalizing current data may be used to determine a treatment endpoint, the endpoint of a treatment zone, and/or to plan an effective IRE and/or H-FIRE treatment.

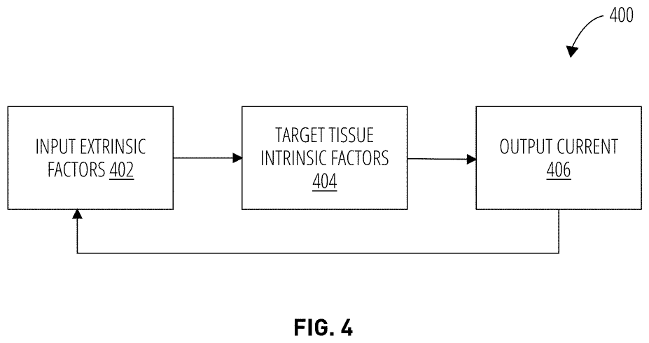

Intrinsic factors are associated with target tissue properties including tissue type, cell size, cell homogeneity, tissue perfusion levels, tissue conductivity and temperature. Extrinsic factors are not related to tissue characteristics but do impact tissue response to the ablation therapy. Extrinsic factors are related to the delivery of electrical fields to the tissue include probe configuration, ablation volume, applied voltage, and specific pulse parameters, among others. The relationship between intrinsic/extrinsic factors and current is conceptually depicted in FIG. 4. In particular, FIG. 4 depicts a feedback system 400 illustrating how extrinsic factors result in the generation of electrical energy. For example, feedback system 400 shows input extrinsic factors 402, which when applied to target tissue (e.g., target tissue 112) having target tissue intrinsic factors 404 results in output current 406. In other words, measured current inherits all the unique and complex intrinsic and extrinsic characteristics that defines a tissue response to electrical energy. Although electrical conductivity is an important property and affects results of an electroporation treatment, it is highly dependent on intrinsic properties which might vary during the procedure. However, current inherits all these complexities and reflects what electrical energy does to tissue without assumptions, error propagation or oversimplification which might be needed for to determine electrical conductivity.

The relationship between current, the intrinsic factors, and the extrinsic factors can be expressed mathematically. IRE using a monophasic waveform, H-FIRE using a biphasic waveform, and or RE using a standard RE waveform is represented by Equation 2. Equation 3 describes current dependence on shape factor and electrical conductivity, which are products of the electrode placement/shape and nature of the target tissue accordingly. Where L=length of probe exposure; r=radius of the probe; V=voltage across the probes; d=distance between probes; t=total timing period; S=shape factor; σ(x,y,z,t)=electrical conductivity of tissue;

k(x,y,z,t)=thermal conductivity of tissue; ω=blood perfusion; Cₚ=specific heat of tissue; X=anisotropic factor; waveform=polarity, pulse width.

I[A]=f(L,V,d,S,t,σ(x,y,z,t),k(x,y,z,t),Cₚ,ω,X,IRE/H-FIRE/RE waveform . . . ) EQUATION 2

I[A]=S(L,d,r,X,etc.)×σ(ω,K,Cₚ,X,etc.)×V EQUATION 3

A benefit to the present disclosure, or said differently, to representing current using equations given herein and normalizing the current to compare current across treatments is that the present disclosure can be used to simplify the impact of these numerous variables associated with ablation volume from IRE and H-FIRE treatments. In one embodiment, the lack of interaction between the current trend for different voltages suggests that the success of IRE is independent of the tested voltages used in treatment planning. In one embodiment, the normalization of current begins with the first pulse of an ablation procedure and upon the initial current which is measured during the first pulse. The normalized current calculation occurs continuously throughout the treatment as the treatment progresses. The normalized current calculation should be treated for individual probe pairs, as each probe pair has its individual characteristics (i.e., exposure, spacing, voltage, etc.).

As discussed herein, the normalization of current inherently accounts for changes to specific extrinsic factors or intrinsic factors for a single procedure, for different procedures of the same patient, or for different procedures for different patients.

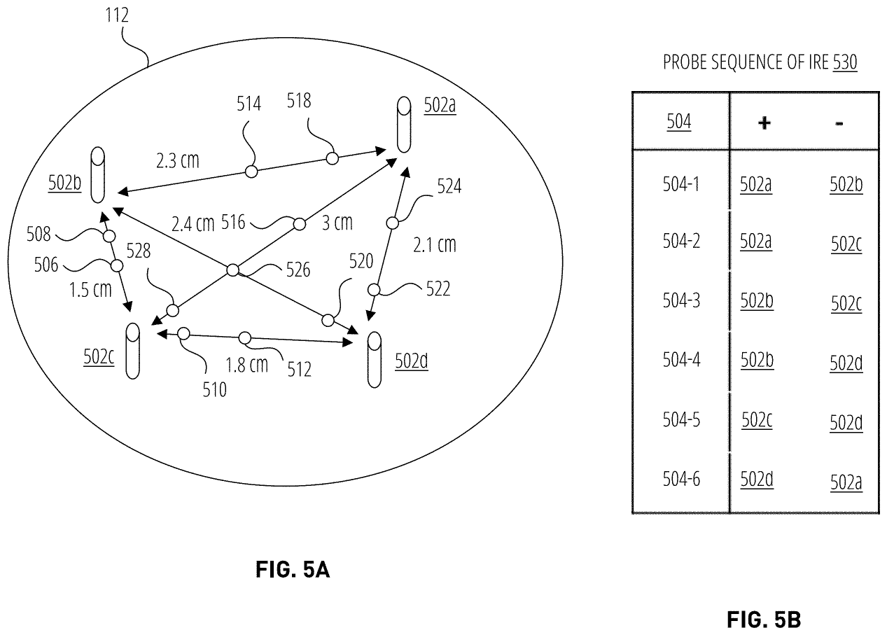

In one embodiment, as shown in FIG. 5A and FIG. 5B, a series of electrical pulses are delivered between a series of four probes. For example, probe 502a, probe 502b, probe 502c, and probe 502d, which can be probe 108 depicted in FIG. 1A and FIG. 1B, and which probes can further be grouped into pair of probes 504. The probes 502a, 502b, 502c, and 502d can be inserted into target tissue 112. The probes can be placed a variety of distances apart. The distances depicted in FIG. 5A are shown for example only. In general, the distance between probes may depend upon the type of target tissue 112, the size or area of target tissue 112, the total number probes used in the therapy. A clinician can place probe 108 into body 110 such that the probes (or electrodes) of probe 108 are inserted into desired locations within or proximate to target tissue 112. The normalization of current can be derived from this specific pulse paradigm (and transferred to a treatment database as described in more detail below) with extrinsic factors or intrinsic factors, such as the number of probes, specific probe placement, electrode exposure, the number of pulses delivered between each probe pair, or specific cycling of probe activation inherent within the normalized current.

In another embodiment, different pulse paradigms may be applied and extrinsic factors and/or intrinsic factors for each of these various pulse paradigms will be inherent in the normalized current. For example, regardless of the specific pulse paradigm used by a physician, the normalization of current will inherently account for the differences between the extrinsic factors and intrinsic factors across the following pulse paradigms: (i) a non-cycled pulse paradigm 100 pulses were delivered per electrode pair for a total number of 600 pulses to the target tissue; (ii) a cycled pulse paradigm (5 pulse cycle, 0 second delay scheme), where 20 pulses were delivered per electrode pair, yield 120 total pulses were delivered per electrode pair, yield 120 total pulses per cycle and, again, a total of 600 pulses to the target region; (iii) a cycled pulse paradigm (5 pulse cycle, 0s delay scheme), where 20 pulses were delivered per electrode pair, yield 120 total pulses per cycle and, again, a total of 600 pulses to the target region with an enhanced electrode pair activation pattern such that no single electrode was activated more than two consecutive times; (iv) a non-cycled pulse Paradigm 100 bursts of pulses were delivered per electrode pair for a total number of 600 bursts of pulses to the target tissue; (v) a cycled pulse paradigm (5 pulse cycle, 0s delay scheme), where 20 burst of pulses were delivered per electrode pair, yield 120 total burst of pulses per cycle and, again, a total of 600 bursts of pulses to the target region; (vi) an asymmetric bipolar waveform and/or monopolar waveform where the positive pulses and/or negative pulses have different durations (each pulse duration ranging between 0.25 μs to 2 μs); or (vii) an asymmetric bipolar waveform where the intrapulse delay varies or where there is no intrapulse delay.

An ablation therapy protocol (or treatment) can include applying a series of voltage pulses via each of the pairs from the pair of probes 210. In some examples, between 10 and 100 voltage pulses can be delivered via each probe pair. With some ablation therapy protocols, voltage pulses are applied via the pair of pair of probes 504 in a sequential order. More particularly, all voltage pulses are applied via the first pair of probes 510-1, followed by the second pair of probes 510-2, etc. Further, applying voltage pulses can be repeated over a number of rounds. For example, a hypothetical ablation therapy protocol could include applying a specific number of voltage pulses having a specific magnitude via the first pair of probes 510-1, applying the specific number of voltage pulses having the specific magnitude via the second pair of probes 510-2, and so forth until the last pair of probes 510-6. This could be referred to as a first round of treatment. A therapy could include multiple rounds. The voltage and the number of pulses need not be the same between rounds. A user may physically move, realign, and/or reposition the placement of the probes 108 within a patient (or for surface electrodes on a patient) between rounds.

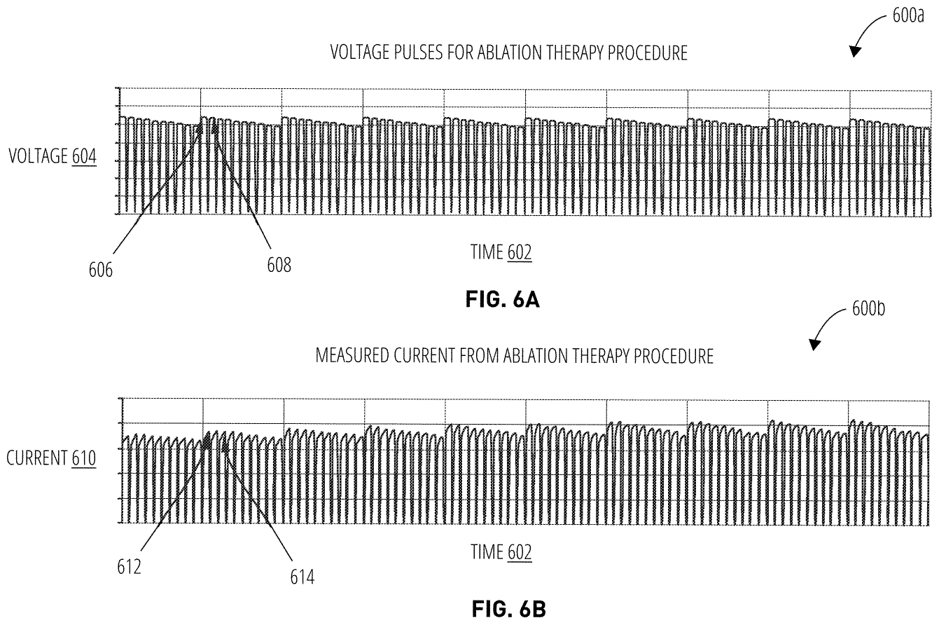

Referring back to FIG. 1A and FIG. 1B, voltage source 104 can be any of a variety of energy sources capable of generating a voltage potential between respective positive and negative electrodes (not shown). Voltage source 104 can apply the generated voltage potential as a series of pulses to target tissue 112 via probe 108. For example, FIG. 6A depicts a plot 600a showing a series of voltage pulses for an ablation therapy procedure where time 602 is represented on the X axis and voltage 604 on the Y axis. It is noted that the time 602 and voltage 604 are not depicted to scale in this figure. The series of voltage pulses include a number of voltage pulses. For example, individual voltage pulse 606 and voltage pulse 608 are called out.

FIG. 3B illustrates a plot 600b showing a series of measured current associated with voltage pulses applied in an ablation therapy where time 602 is represented on the X axis and current 610 on the Y axis. Like in plot 600a of FIG. 6A, time 602 and current 610 are not depicted to scale. As depicted, the series of measured current values associated with voltage pulses applied in an ablation therapy include current for a number of pulses, which each correspond to one of the voltage pulses from the series of voltage pulses depicted in plot 600a. For example, individual current pulse 612 and current pulse 614 are called out. As described more fully herein, ablation therapy device 102 can measure current produced by applying the series of voltage pulses depicted in plot 600a to target tissue 112, resulting in the series of measured current 312. Examples directed to normalizing this measured current and techniques and devices configured to respond to the normalized current are more fully described below.

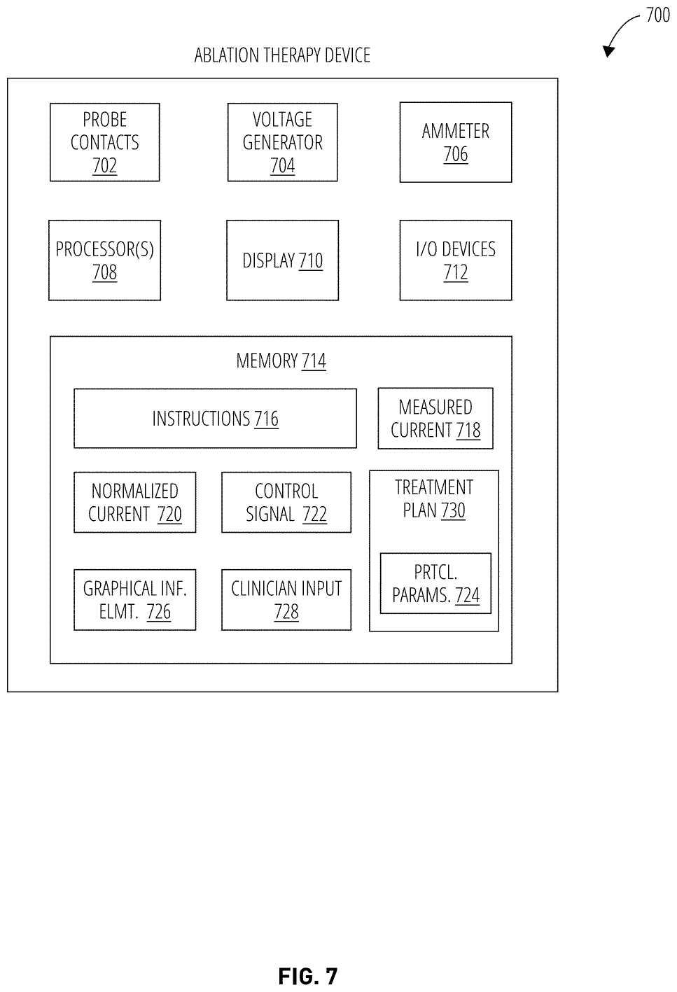

FIG. 7 illustrates an ablation therapy device 700. In some examples, ablation therapy device 700 can be implemented as the ablation therapy device 102 of FIG. 1A. Ablation therapy device 700 includes probe contacts 702, voltage generator 704, ammeter 706, processor(s) 708, display 710, input and/or output devices (I/O devices 712), and memory 714. Probe contacts 702 can include mechanical coupling mechanisms to secure probes (e.g., probe 108, or the like) to the ablation therapy device 700. Further probe contacts 702 includes electrical contacts to provide electrical conductivity between voltage generator 704 and electrodes in a probe. Voltage generator 704 can be any of a variety of voltage generators arranged to generate voltage pulses as described herein. A discussion of voltage generators and voltage pulses was given above and is not repeated here for brevity. Ammeter 706 can be any of a variety of current measuring devices electrically coupled to voltage generator 704 and/or probe contacts 702 such that current produced by application of voltage pulses to target tissue can be measured.

The processor(s) 708 can include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor(s) 708 may include graphics processing portions and may include dedicated memory, multiple-threaded processing and/or some other parallel processing capability. In some examples, the processor(s) 708 may be an application specific integrated circuit (ASIC) or a field programmable integrated circuit (FPGA). In some implementations, the processor(s) 708 may be circuitry arranged to perform particular computations, such as, related to artificial intelligence (AI) or graphics. Such circuitry may be referred to as an accelerator. Processor(s) 708 can include multiple processors, such as, for example, a central processing unit (CPU) and a graphics processing unit (GPU).

The memory 714 can include both volatile and nonvolatile memory, which are both examples of tangible media configured to store computer readable data and instructions to implement various embodiments of the processes described herein. Other types of tangible media include removable memory (e.g., pluggable USB memory devices, mobile device SIM cards), optical storage media such as CD-ROMS, DVDs, semiconductor memories such as flash memories, non-transitory read-only-memories (ROMS), dynamic random access memory (DRAM), NAND memory, NOR memory, phase-change memory, battery-backed volatile memories, networked storage devices, and the like.

The memory 714 may include a number of memories including a main random access memory (RAM) for storage of instructions and data during program execution and a read only memory (ROM) in which read-only non-transitory instructions are stored. Memory 714 may include a file storage subsystem providing persistent (non-volatile) storage for program and data files. Memory 714 may further include removable storage systems, such as removable flash memory.

The memory 714 may be configured to store the basic programming and data constructs that provide the functionality of the disclosed processes and other embodiments thereof that fall within the scope of the present invention. Memory can store instructions 716, measured current 718, normalized current 720, control signal 722, protocol parameters 724, graphical information element 726, clinician input 728, treatment plan 730. During operation, processor(s) 708 can read instructions 716 from memory 714 and can execute the instructions 716 to implement embodiments of the present disclosure. Memory 714 may also provide a repository for storing data used by the instructions 716 or data generated by execution of the instructions 716. Such embodiments and examples of the data structures depicted in this figure are given in more detail below.

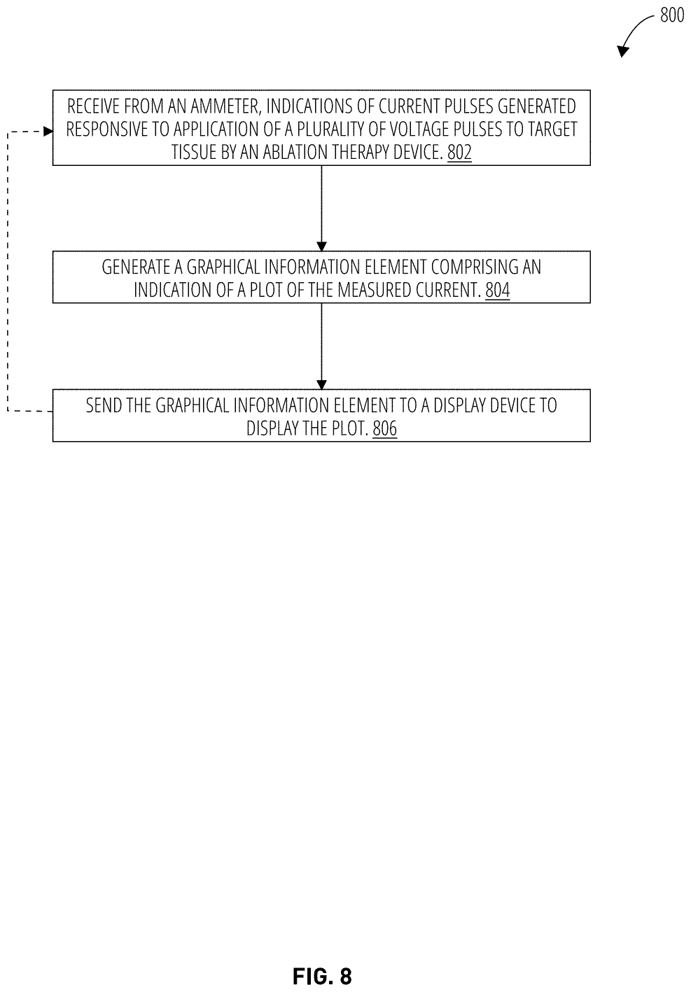

FIG. 8 depicts a routine 800 that may be implemented by an ablation therapy device according to examples of the present disclosure. At block 802 “receive from the ammeter, indications of current pulses generated responsive to application of a plurality of voltage pulses to the target tissue by an ablation therapy device” indications of current measured at an ammeter can be received. For example, in executing instructions 716 processor(s) 708 can receive measured current 718 from ammeter 706.

At block 804 “generate a graphical information element comprising an indication of a plot of the measured current” graphical data comprising an indication of a plot of the measured current 718 can be generated. For example, in executing instructions 716 processor(s) 708 can generate graphical data (e.g., display frames, or the like) including indications of a plot representing the current measured at block 802 (e.g., measured current 718). The graphic data can be stored in memory 714 as graphical information element 726.

At block 806 “send the graphical information element to a display device to display the plot” the ablation therapy device can send the graphical information element 726 to display 710 to display the plot indicated by the graphical information element 726. For example, in executing instructions 716 processor(s) 708 can send the graphical information element 726 to display 710 and display 710 can display the plot indicated by the graphical information element 726.

In some examples, routine 800 can be repeated such that display 710 can be updated with indications of measured current 418 as an ablation therapy treatment progresses. For example, ablation therapy device 102 could implement routine 800 at the conclusion of each round of voltage pulses, at the conclusion of each subset or train (e.g., 5 pulses, 10 pulses, 20 pulses, or the like) of voltage pulses. As such, display 710 can be updated with indications (e.g., via plots, or the like) of measured current 718 as the ablation therapy progresses providing feedback to the clinician of the progress of the ablation therapy treatment prior to a conclusion of the specified treatment protocol (e.g., prior to application of all scheduled voltage pulses, or the like).

Further, routine 800 can be repeated individually for each pair of probes or collectively for all probe pairs. For example, routine 800 can be implemented such that a plot depicting current from one pair of probes can be generated at block 804 and can be repeated such that another plot depicting current from another pair of probes can be generated at block 804. In some examples, both plots can be displayed on display 710. In other examples, a single plot depicting current from multiple pairs of probes can be generated.

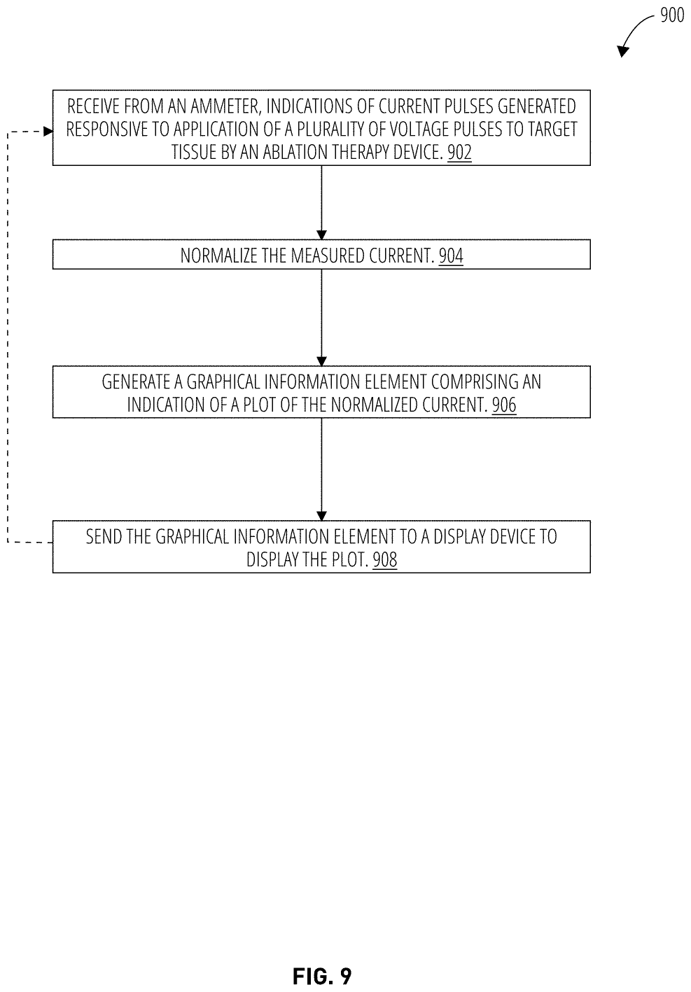

FIG. 9 depicts a routine 900 that may be implemented by an ablation therapy device according to examples of the present disclosure. At block 902 “receive from the ammeter, indications of current pulses generated responsive to application of a plurality of voltage pulses to the target tissue by an ablation therapy device” indications of current measured at an ammeter can be received. For example, in executing instructions 716 processor(s) 708 can receive measured current 718 from ammeter 706.

At block 904 “normalize the measured current” the measured current can be normalized. For example, in executing instructions 716 processor(s) 708 can normalize the measured current 718 to generate normalized current 720. In some examples, processor(s) 708 can execute instructions to normalize the measured current for voltage. Said differently, processor(s) 708 can execute instructions 716 to normalize measured current 718 to a common reference point, resulting in normalized current 720. With some examples, current can be normalized with any normalization techniques such as linear scaling, clipping, log scaling or Z-score, or other statistical normalization techniques. As a specific example, measured current can be normalized using Equation 4, where I′=normalized current; Io=initial current; I=final current after 10 pulses.

I′=I[A]/I0[A] EQUATION 4

In some examples, at block 804 a rate of change of the normalized current can be derived. For example, processor(s) 708 in executing instructions 716 can determine a rate of change of the normalized current 720 using Equation 5, where RC=rate of change of current; I′=normalized current; t=time.

RC=dI′/dt EQUATION 5

At block 906 “generate a graphical information element comprising an indication of a plot of the normalized current” graphical data comprising an indication of a plot of the normalized current can be generated. For example, in executing instructions 716 processor(s) 708 can generate graphical data (e.g., display frames, or the like) including indications of a plot representing the normalized current 720. The graphic data can be stored in memory 714 as graphical information element 726. With some examples, the graphical information element 726 can include indications of a plot depicting the normalized current 720, a derived rate of change of the normalized current 720, or both the normalized current 720 and a derived rate of change of the normalized current 720.

At block 908 “send the graphical information element to a display device to display the plot” the ablation therapy device can send the graphical information element 726 to display 710 to display the plot indicated by the graphical information element 726. For example, in executing instructions 716 processor(s) 708 can send the graphical information element 726 to display 710 and display 710 can display the plot indicated by the graphical information element 726.

In some examples, routine 900 can be repeated such that display 710 can be updated with indications of normalized current 720 as an ablation therapy treatment progresses. For example, ablation therapy device 102 could implement routine 900 at the conclusion of each round of voltage pulses, at the conclusion of each subset or train (e.g., 5 pulses, 10 pulses, 20 pulses, or the like) of voltage pulses. As such, display 710 can be updated with indications (e.g., via plots, or the like) of normalized current 720 as the ablation therapy progresses providing feedback to the clinician of the progress of the ablation therapy treatment prior to a conclusion of the specified treatment protocol (e.g., prior to application of all scheduled voltage pulses, or the like).

Further, routine 900 can be repeated individually for each pair of probes or collectively for all probe pairs. For example, routine 900 can be implemented such that a plot depicting normalized current from one pair of probes can be generated at block 906 and can be repeated such that another plot depicting normalized current from another pair of probes can be generated at block 906. In some examples, both plots can be displayed on display 710. In other examples, a single plot depicting normalized current from multiple pairs of probes can be generated.

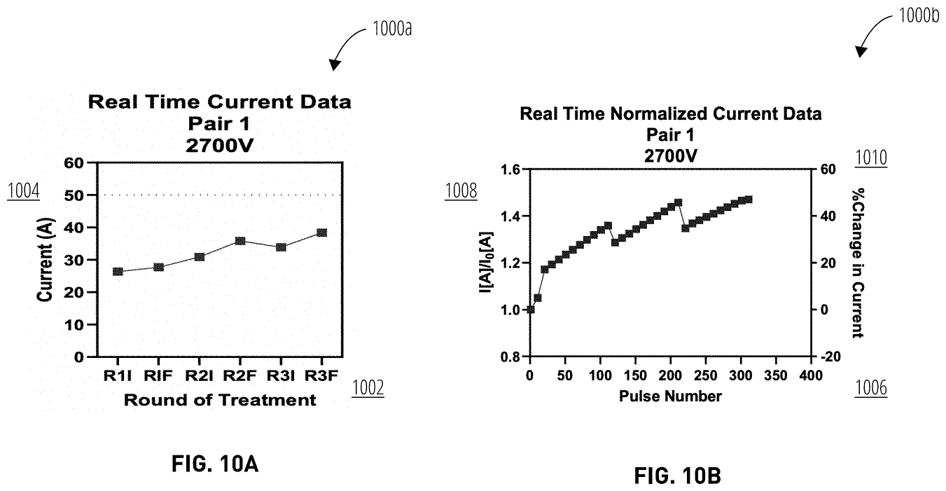

FIG. 10A shows a plot 1000a depicting real time current data for a probe pair over a number of rounds of treatment 1002 of an ablation therapy treatment. A treatment round is a group of pulses delivered between a pair of probes before moving to the next pair. For example, in one embodiment round 1 range between 10 pulses to 20 pulses, and subsequent rounds are between 70 pulses to 100 pulses. However, the number of pulses in each round could be different depending on the physician's specific treatment protocol. The rounds of treatment 1002 are depicted on the X axis while the current 1004 is depicted on the Y axis. For example, RH represents round 1 initial pulse and R1F represents the round 1 final pulse before moving to other probe pairs, and R2I represents round 2 initial pulse and R2F represents the round 2 final pulse before moving to other probe pairs. With some examples, routine 800 can generate graphical information element 726 including indication of a plot like plot 1000a.

In one embodiment, the specific and/or predominate mechanism of action causing or resulting in changes at the cellular level in each specific zone(s), with reference to FIG. 8, is depicted in Table 3 below: As noted, both intrinsic and extrinsic factors may cause variation in tissue response from one probe pair to another. To that end, FIG. 10B shows a plot 1000b depicting real time normalized current data for a probe pair over a number of voltage pulses of an ablation therapy treatment. The voltage pulse 1006 are depicted on the X axis while the normalized current 1008 and the percent rate of change 1010 are depicted on the Y axis. With some examples, routine 900 can generate graphical information element 726 including indication of a plot like plot 1000b. The data depicted in FIG. 10A, which reflects the unique current I conductivity profile for a specific probe pair in a specific tissue type, can be used by the clinician to adjust pulse delivery to that probe pair to account for these variations. Whereas FIG. 10B illustrates the normalization of the current data depicted in FIG. 10A, and as such provides the user with data that can be used for treatment planning purposes and real time monitoring of treatment progress, as described in more detail below.

An IRE and/or H-FIRE treatment procedure can include multiple therapy zones. In general, as used herein, a therapy zone is region associated with a particular treatment characteristic (e.g., a trend in tissue conductivity, a trend in measured current, a trend in normalized current, or the like). A therapy zone is representative of the specific mechanism(s) of action causing or resulting in changes at the cellular level due to the delivery of the electrical pulses. For example, a therapy zone may comprise any of the following, RE, IRE, H-FIRE, thermal ablation, electrolysis, RE and IRE and H-FIRE, IRE and HFIRE, IRE and HFIRE and thermal ablation and electrolysis, and/or thermal ablation and electrolysis. A particular therapy zone may be associated with a particular tissue response, for example irreversible electroporation of tissue within the target zone. Some clinicians desire to conclude an ablation therapy treatment in one of these therapy zones or at a transition between selected therapy zones. However, given conventional ablation therapy tools and treatment protocols there is not a way to determine which therapy zone the treatment is currently in or to predict how the treatment will progress through the therapy zones.

In one embodiment, zone one, zone two, and zone three can have different intensity levels of IRE and/or HFIRE. For example, zone one has a lower IRE and/or H-FIRE intensity than zone two and zone three, whereas zone three has a high IRE and/or H-FIRE intensity than zone two and zone one. The different IRE and/or H-FIRE intensities affect the tumor microenvironment with different mechanisms of action.

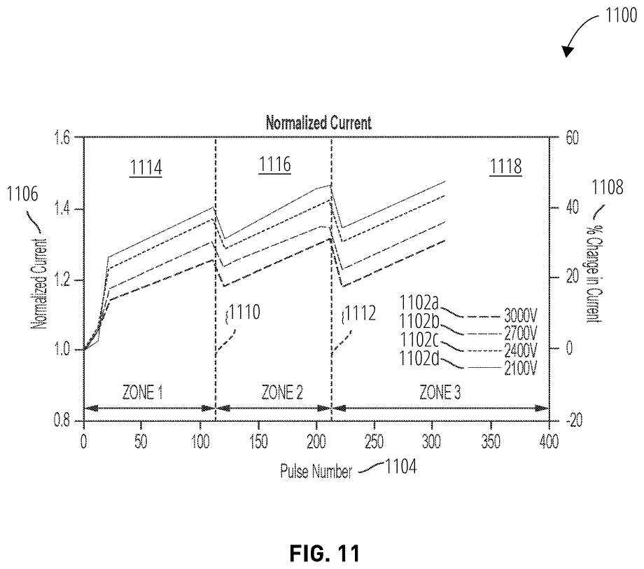

The specific and/or predominate mechanism of action causing or resulting in changes at the cellular level in each specific zone(s), with reference to FIG. 11, is depicted in Table 3 below.

TABLE 3

| | Zone Zero | Zone One | Zone Two | Zone Three |
|---|---|---|---|---|
| Potential | RE, | RE, | RE, IRE, | Thermal |
| mechanism(s) | IRE, and/or | IRE and/or | H-FIRE, | ablation |
| of action | H-FIRE | H-FIRE | thermal | and/or |
| | | | ablation, | electrolysis |
| | | | and/or | |
| | | | electrolysis | |
| Predominate | RE | IRE and/or | IRE and/or | Thermal |
| mechanism(s) | | H-FIRE | H-FIRE | ablation |
| of action | | | | and/or |
| | | | | electrolysis |

The specific and/or predominate mechanism of action of cell death in each zone will be dependent on certain extrinsic and/or intrinsic factors (i.e., tissue type, conductivity of the target area, pulse paradigm, specific pulsing patters, applied voltage).

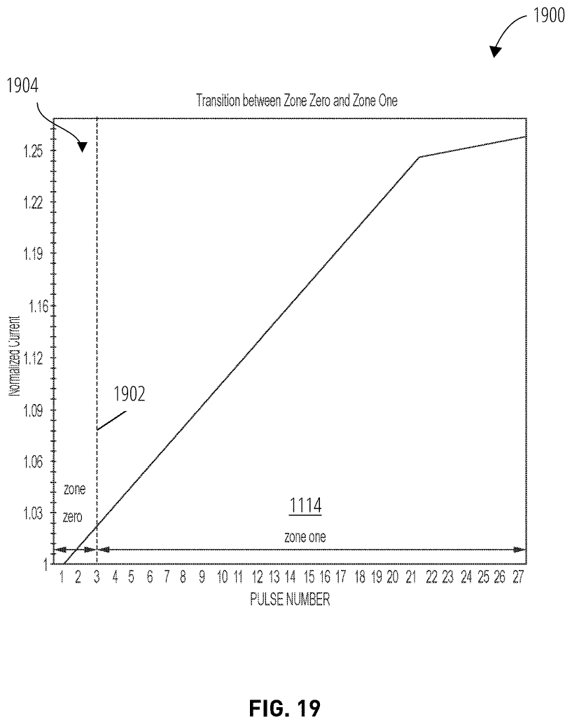

In one embodiment, in zone zero the predominate mechanism of action is RE with potentially IRE and/or H-FIRE effects. The specific transition point between mechanism of action predominantly RE and predominantly IRE and/or H-FIRE (for one example, see FIG. 19) may be impacted by the heterogeneity of electric field (e.g., using needle electrodes) and/or the heterogeneity of tissue (i.e., different cell types: epithelial, healthy, cancerous). For example, some cells might need more pulses to achieve the voltage gradient sufficient to achieve IRE and/or H-FIRE (i.e., greater than 800 v/cm) and for cells in target area to form irreversible pore formation. In zone one, the mechanism of action transitions to predominantly IRE and/or H-FIRE. In zone one, the vasculature structure may be preserved for continued blood supply and oxygenation to the target site. The extent of apoptotic response as a result of the IRE and/or H-FIRE would likely induce anti-inflammatory cell death in cancerous cells while preserving the extracellular membrane structure. In zone two, the mechanism of action transitions to include IRE, H-FIRE, thermal ablation and/or electrolysis. Zone two would likely represent a combination of anti-inflammatory and proinflammatory cell death mechanisms where close to the electrodes and high intensity electrical fields, the other ablation modalities dependent damage will contribute to necrosis and pyroptosis. During zone two substantially the entire tumor microenvironment is going through non-homogenous cell death mechanisms from center to periphery. The regions of zone two that are undergoing apoptosis and necroptosis may contain a microenvironment to induce an anti-tumor immune response; whereas the rest of zone two is undergoing damage from other ablation modalities which may stop angiogenesis and hence provide more oxygen for the coming infiltrated T cells. However, since the supply of oxygen depends on the number of vasculature and perfusion rate, the tissue with target area undergoes hypoxia. The impact of zone 3 on an extracellular membrane is significant and due to the plasma membrane disruption, the proinflammatory signals are activated and so the innate and adaptive immune responses. This scenario would serve a case where the adaptive immune system is not yet activated and therefore systemic immune response (cold tumors, immunosuppressed) needs to be induced. In one non-limiting example, after a few hours to days, the angiogenesis and vasculogenesis will occur as a natural healing process due to the injury, and therefore there will be enough oxygen supply for the infiltrated cells which can be provided by other adjuvant therapies to the tumor microenvironment.

In one embodiment (not shown), the ablation device described herein comprises a sensor feedback mechanism to better define or identify electrolysis zones (i.e., transition with thermal and IRE and/or H-FIRE) and electrolysis zone. The sensory feedback mechanism is used to monitor for electrolysis factors. Electrolysis factors comprise tissue properties linked to electrolysis (i.e., PH changes). Electrolysis is a chemical ablation mechanism of action, and the extent of ablation is a function of the concentration of the chemical species and the exposure time to such chemicals. The sensory feedback mechanism may comprise a sensor to monitor PH levels or changes, and/or temperature. For example, the electrode may be operatively coupled to the sensory feedback mechanism and is configured to monitoring electrolysis factors. The system can then compare the monitored electrolysis factor information with the normalized current value to provide feedback to a user to identify the point in time in which a target tissue undergoes electrolysis. The monitored electrolysis factor information may also be included in a treatment database to aid in the machine learning aspects of this disclosure (as described in more detail below).

In one embodiment, the device provides intra-treatment feedback in real-time to a clinician regarding which therapy zone the treatment is in as well as to predict progression of the treatment through the therapy zones. FIG. 11 illustrates a plot 1100 showing normalized current trend lines 1102a, 1102b, 1102c, and 1102d where number of pulses 1104 is represented on the X axis and both a change in normalized current 1106 and a percentage change in normalized current 1108 are on the Y axis. As shown, the normalized current 1106 has an initial value of 1.0 for all four trend lines 1102a, 1102b, 1102c, and 1102d. In one non-limiting example, as pulses are delivered, the normalized current 1106 continues to increase for all trend lines 1102a, 1102b, 1102c, and 1102d until approximately 120 pulses have been delivered. The transition point 1110 from zone one 1114 to zone two 1116 is defined by a drop in the normalized current 1106 value for all trend lines 1102a, 1102b, 1102c, and 1102d. Although the normalized current values and transition point 1110 between zones will vary from procedure to procedure, the drop in normalized current is a reliable indicator of a change in the mechanism of cell death. For example, zone one 1114 may indicate to the user that cell death of the target tissue is primarily due to irreversible electroporation. Transition point 1110 to zone two 1116 may indicate that mechanism of cell death is transitioning from primarily IRE to another ablation modality (such as thermal ablation). This information can be used by a clinician for several different purposes, including, but not limited to, determining if additional pulses should be delivered, determining the type of cell death occurring in the target area, determining the potential immune response the body may have as a result of additional electrical pulses to be delivered, and/or determining the completeness of the IRE and/or H-FIRE treatments. Plotting of the rate of change in normalized current and/or the percentage change in current, as shown in FIG. 11, may also be used to assist the clinician in identifying the onset of reversible electroporation, irreversible electroporation, thermal ablation, and electrolysis in the target cell zone. For the sake of clarity, zone zero is often not depicted in the figure. However, FIG. 19 includes zone zero (as described in more detail below).

FIG. 11 may be generated to provide a user with feedback on a planned or recommended ablation procedure and where, within the ablation procedure the therapy zones might progress based on planned procedure protocol parameters. For example, zone one 1114 may comprise a predominantly IRE and/or H-FIRE mechanism of action, resulting in a sudden rise in normalized current depicted in trend lines 1102a, 1102b, 1102c, and 1102d.

During use, a treatment plan (e.g., treatment plan 730) may be generated where the treatment plan includes protocol parameters 724 for an IRE and/or H-FIRE procedure. In particular, the treatment plan 730 can be generated based on clinician input 728 comprising indications of an immunoscore and/or immunity type of a tumor to be treated as well as other information depicted in the figures and described herein, such as, tumor size, desired margin, or the like. For example, where a treatment plan 730 including protocol parameters 724 associated with a voltage of 2700V (trend line 1102b) a graphical display (like the plot 1100) can be generated to display the normalized current and percent change in current for the selected parameter(s) 2700 V, as well as the transition between zones. For example, where the treatment plan 730 is generated comprising an intensity associated with zone one 1114, an indication (e.g., graphical, audible, or otherwise) that a zone one 1114 treatment has been completed may be generated. Responsive to such an indication, the user may decide to continue the treatment and the system (e.g., ablation therapy device 700, or the like) can receive an indication to continue such treatment.

Transition zones indicated by points 1110 and 1112 suggests to a user a change in electrical properties of the tissue. Zone two 1116 is still predominantly IRE and/or HFIRE mechanism of action, but the effect of other ablation modalities (i.e., thermal ablation and/or electrolysis) are not negligible when it comes to cell death mechanisms and consequently triggering different immune reaction. Transition zone indicated by point 1112 shows a transition between zone two 1116 and zone three 1118. Within zone three 1118 the effect of other ablation modalities (i.e., thermal ablation and/or electrolysis) is a substantial part of the treatment which trigger different cell death pathways and immune response.

The normalized current plot shown in FIG. 11 also illustrates the maximum percent change in normalized current (PCNC) that the target tissue reaches in addition to the maximum PCNC achieved as the treatment progresses from one zone to another as described herein. Using the 2100V trend line 1102d (which represents the largest change in PCNC depicted in the plot), the PCNC at pulse 120 is approximately 40%, rising to approximately 50% at pulse 220 and returning to a PCNC of approximately 50% at pulse 325. Furthermore, again using the 2100V trend line 1102d the PCNC increased 40% within zone one 1114, an incremental increase of 10% within zone two 1116, and no significant incremental increase within zone three 1118, with the total PCNC increases about 50%. Furthermore, at pulse 120, where the electrical energy impact on the target tissue transitions from predominately IRE to other ablation modes, the PCNC is at 40% increase from the baseline. This suggests that the percentage changes from the baseline for electrical conductivity will be limited for transition or thermal zones. When switching to the next probe pair, the ablated tissue will have enough time to get rid of the accumulated heat by conduction or convection. In other words, the rise in current and conductivity within these zones will be diminished after the completion of pulse delivery, solely because these electrical conductivity changes are due to the temperature changes and not due to electroporation (−2-3% change in electrical conductivity/° C.).

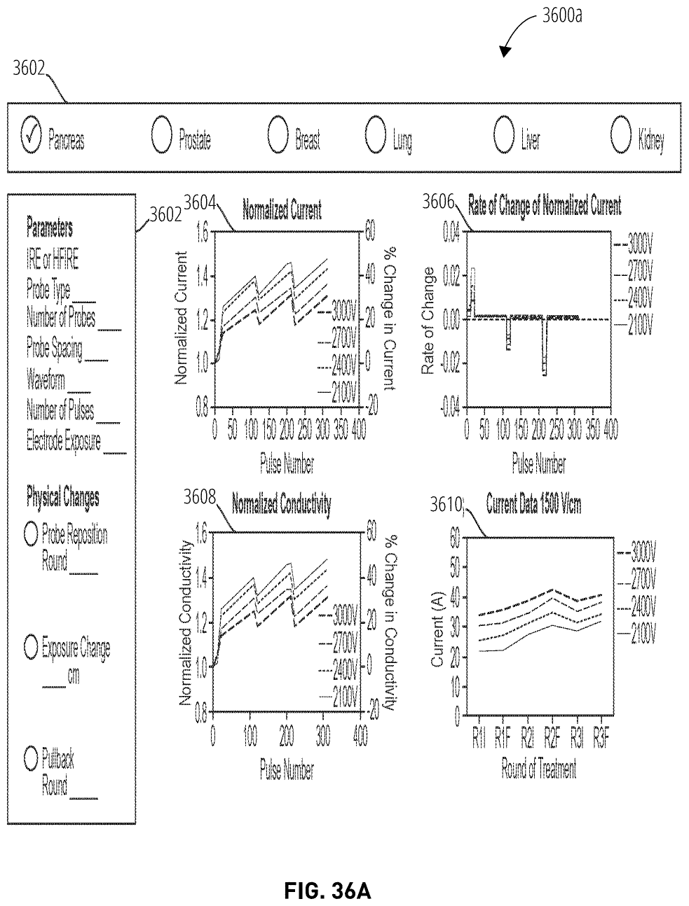

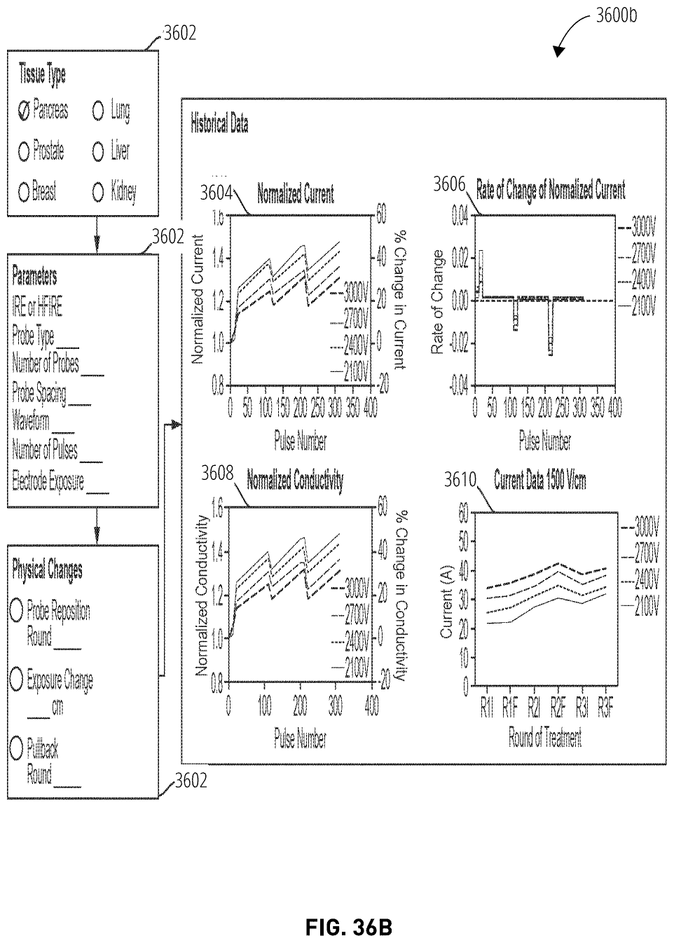

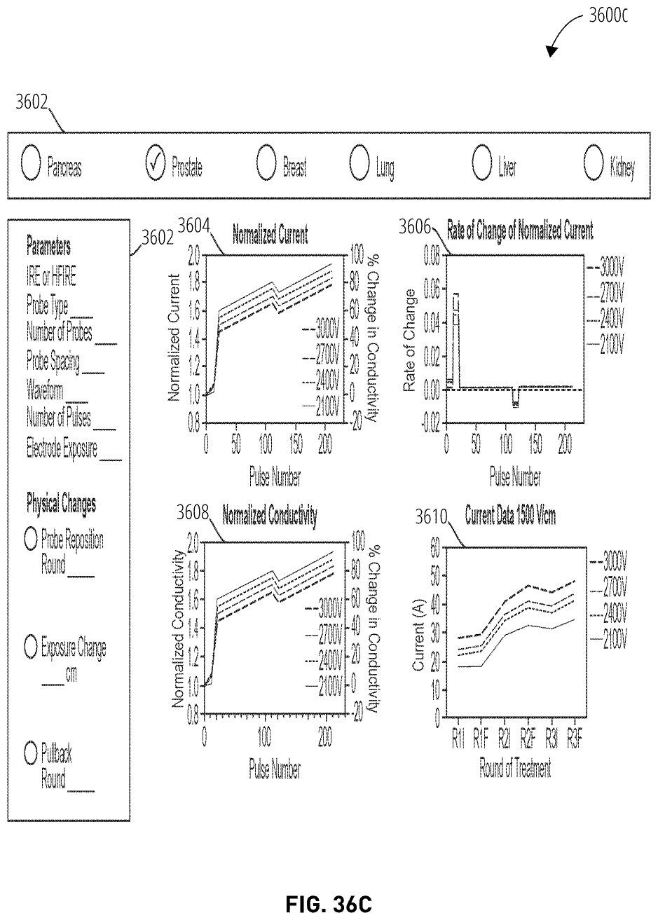

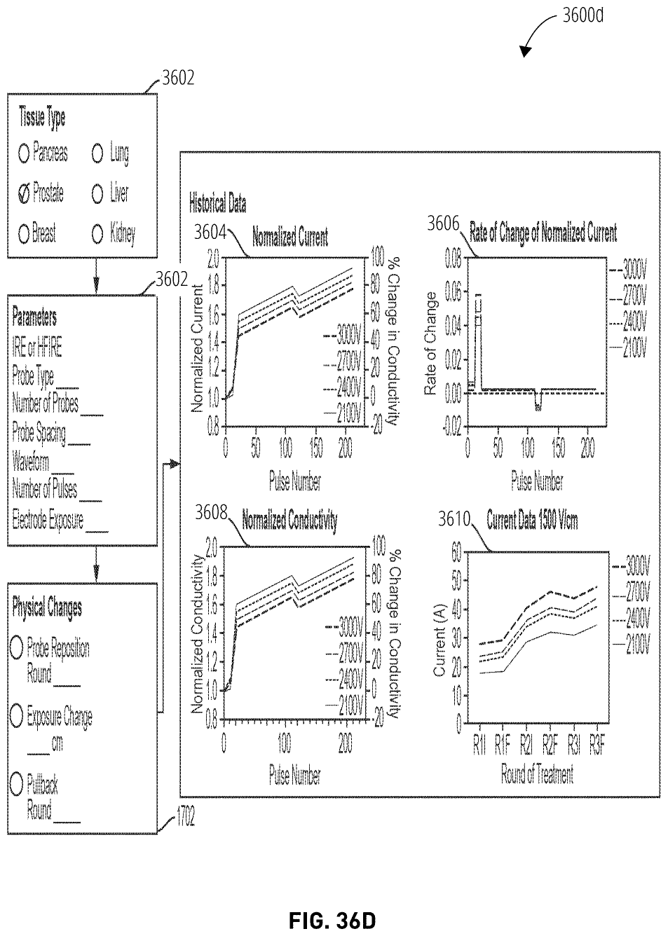

Referring to FIG. 36B, the Percent Change in Normalized Conductivity (PCNCJ) may also be analyzed in a similar manner. Although the maximum of both PCNC and PCNCJ values will vary based on the type of tissue being treated, these calculations may be used for pre-treatment planning purposes or to estimate dynamic conductivity of the target tissue in real time.

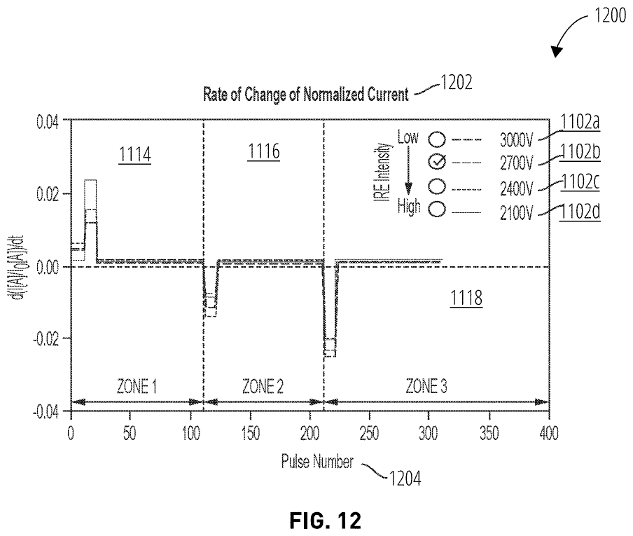

FIG. 12 depicts an example plot 1200 in which the user may use a hypothetical normalized current measurement as a parameter for treatment planning. The user may use the data stored in the treatment database (described in detail below) to perform treatment planning. The data to support the treatment planning plot 1200 parameters comprise the rate of change of the hypothetical normalized current measurements 1202, pulse numbers 1204, hypothetical IRE intensity, and hypothetical zone one 1114, hypothetical zone two 1116, and hypothetical zone three 1118 may be derived from the treatment database as described in detail below. During use, the user may select a tissue type (not shown) and receive a recommendation for an intensity of treatment (e.g., zone one 1114, zone two 1116, zone three 1118, etc.). During the treatment, the system can be arranged to display the plot 1200 indicating the rate of change of hypothetical normalized current (e.g., 2300 V, etc.) the recommended IRE intensity and the transition between hypothetical zones (e.g., zone one 1114 and zone two 1116, etc.) identified. This allows the user to see how various treatment parameters will potentially impact the rate of change of normalized current, the IRE intensity, and the transition between hypothetical zones.

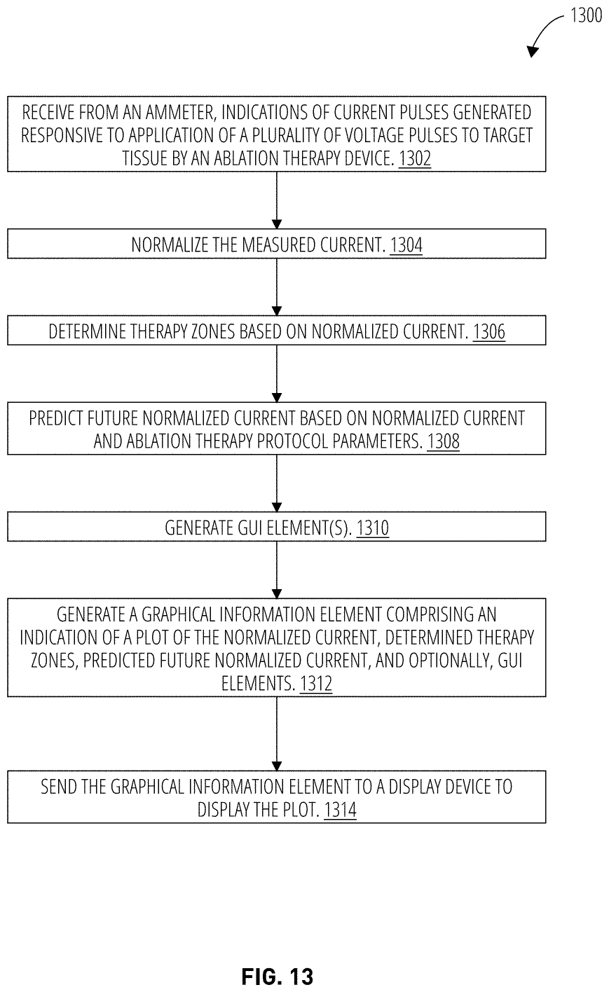

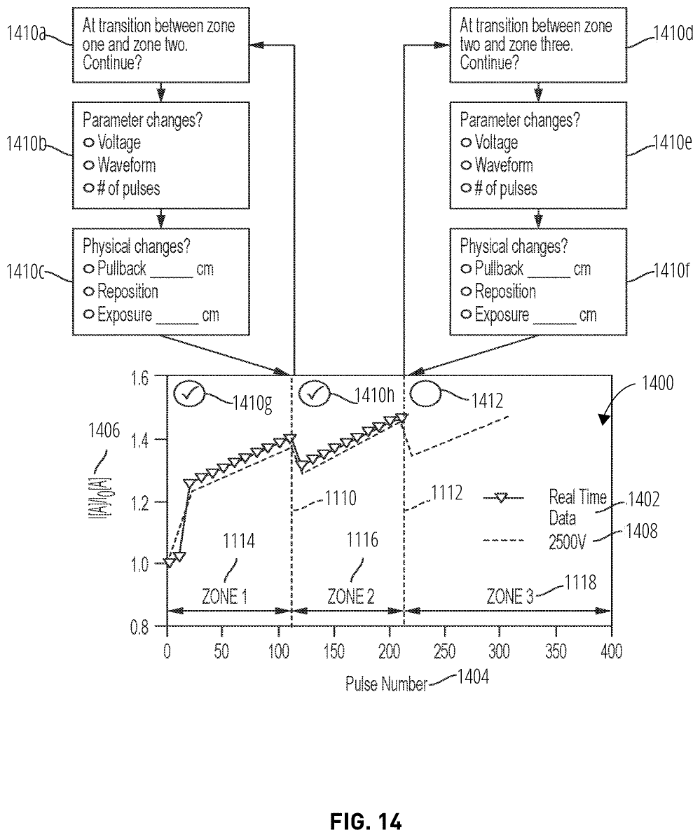

FIG. 13 depicts a routine 1300 that may be implemented by an ablation therapy device according to examples of the present disclosure. In some examples, routine 1300 can be implemented to generate graphical data for display on display 710. For example, FIG. 14 depicts an example plot 1400 that may be generated based on routine 1300. The routine 1300 of FIG. 13 and plot 1400 of FIG. 14 are described together herein. The system may be capable of automatically stopping or pausing the delivering of electrical pulses upon the completion of a therapy zone. For example, prior to the delivery of electrical pulses, the user may select an option so the voltage source 104 is to stop or pause the delivery of electrical pulses once the normalized current measurement indicates that the treatment has completed a selected therapy zone or a transition between selected therapy zones.

As discussed herein, the primary mechanism(s) of cell death within the target tissue may be classified in zones based on the trend of the normalized current. For example, the first few pulses (e.g., up to first 5 pulses) the cells undergo predominantly reversible electroporation (discussed in more detail below and shown as “zone zero” in FIG. 19). As the normalized current progresses within zone one 1114 the mechanism(s) of cell death is predominantly irreversible electroporation. As the normalized current transitions between zone one 1114 into zone two 1116 and then progresses within zone two 1116 the mechanism of cell death predominantly transitions between irreversible electroporation to thermal ablation. As the normalized current transitions between zone two 1116 into zone three 1118 and then progresses within zone three 1118 the mechanism of cell death is predominantly continued thermal ablation and/or electrolysis. Therefore, mechanism(s) of cell death occur at different levels of severity and/or different mechanisms of cellular death as normalized current transitions between zone one 1114, zone two 1116, and zone three 1118. FIG. 11 and FIGS. FIG. 36A to FIG. 36D are illustrative examples. Furthermore, when the mechanism of action is predominantly irreversible electroporation the cell death is predominantly apoptotic cell death; whereas when the mechanism of action is predominantly thermal ablation the cell death is predominantly necrotic cell death. When the mechanisms of action of cell death comprises different mechanisms of action (i.e., ire, thermal, electrolysis, and/or a transition between these mechanisms of action), multiple cell death pathways (instead of single dominant cell death pathway) are expected.

Moreover, tissue will undergo apoptotic cell death in a milder excitation (i.e., irreversible electroporation) mechanism of action (non-inflammatory nor PMPs or DAMPs triggered) and undergo necrosis or pyroptosis when exposed to harsher excitation (i.e., thermal ablation, high dose of radiation, and/or chemo-therapy) which will be proinflammatory with induction of PAMPs and DAMPs.

FIG. 14, illustrates a plot 1400 showing a normalized current trend line 1402 where number of pulses 1404 is represented on the X axis and changes in normalized current 1406 the Y axis. As shown, the normalized current 1406 has an initial value of 1.0. As pulses are delivered, the normalized current 1406 continues to increase until approximately 120 pulses have been delivered. The transition point 1110 from zone one 1114 to zone two 1116 is defined by a drop in the value of the normalized current trend line 1402. Although the normalized current values and transition points between zones will vary from procedure to procedure, the drop in normalized current is a reliable indicator of a change in the mechanism of cell death. For example, zone one 1114 may indicate to the user that cell death of the target tissue is primarily due to irreversible electroporation. Transition to zone two 1116 may indicate that mechanism of cell death is transitioning from primarily IRE to another ablation modality. This information can be used by a clinician for several different purposes, including, but not limited to, determining if additional pulses should be delivered, determining the type of cell death occurring in the target area, determining the potential immune response the body may have as a result of additional electrical pulses to be delivered, and/or determining the completeness of the IRE and/or H-FIRE treatments.

Also shown in FIG. 36A to FIG. 36D, the rate of change of the normalized current (electroporation intensity) initially falls below zero at approximately 120 pulses. The start of zone three 1118 of FIG. 14 and the second negative rate of change in FIG. 36A to FIG. 36D both begin after approximately 120 pulses.

As shown in FIG. 14, at the first transition point 1110 a series of GUI elements may be displayed on a display unit to prompt user input. For example, GUI elements GUI element 1410a, 1410b, and 1410c at first transition point 1110 may comprise a query of “at transition between zone one and zone two. Continue?”; a query of “Parameter Change?” (i.e., change to voltage, waveform, or number of pulses); a query of “Physical Changes?” (i.e., a pullback length, reposition probes, or exposure length). Also shown at the second transition point 1112 a series of GUI elements GUI element 1410d, 1410e, and 1410f may be displayed on a display unit to prompt user input. For example, GUI elements at first transition point 1112 may comprise a query of “At transition between zone two and zone three. Continue?”; a query of “Parameter Change?” (i.e., change to voltage, waveform, or number of pulses); a query of “Physical Changes?” (i.e., a pullback length, reposition probes, or exposure length). These GUI elements are non-limiting examples, and it is within the scope of this disclosure other parameters common for ablation procedures that be included as GUI elements.

A threshold number of pulses that triggers both a zone change in the normalized current trend and decrease in rate of normalized current change is not constant but rather will vary based on intrinsic and extrinsic factors. As an example, if all other extrinsic and intrinsic factors assumed equal, tissue with a low baseline conductivity will require more pulses before transitioning to the next zone than higher conductivity tissue. As another example, a probe pair placed further apart will require more pulses before transitioning to a second zone and a negative rate of normalized current change than probe pairs placed closer together.

Referring back to FIG. 13, at block 1302 “receive from the ammeter, indications of current generated responsive to application of a plurality of voltage pulses to the target tissue by an ablation therapy device” indications of current measured at an ammeter can be received. For example, in executing instructions 716 processor(s) 708 can receive measured current 718 from ammeter 706. At block 1304 “normalize the measured current” the measured current can be normalized. For example, in executing instructions 716 processor(s) 708 can normalize the measured current 718 to generate normalized current 720. In some examples, processor(s) 708 can execute instructions to normalize the measured current for voltage. Said differently, processor(s) 708 can execute instructions 716 to normalize measured current 718 to a common reference point, resulting in normalized current 720. Actual normalized current 720 is depicted as normalized current 1408 on the Y axis of plot 1400 versus pulse number of pulses 1404, which are depicted on the X axis of plot 1400.

At block 1306 “determine therapy zones based on normalized current” therapy zones can be based on the normalized current 720. In general, therapy zones and transitions between therapy zones can be defined by a change (e.g., percent increase/decrease, increase/decrease greater than a threshold value, or the like) in the normalized current 720. For example, plot 1400 depicts treatment zone one 1114, treatment zone two 1116, and treatment zone three 1118.

Additionally, transition one point 1110 between zone one 1114 and zone two 1116 as well as transition two point 1112 between zone two 1116 and zone three 1118 are depicted. As a non-limiting example, therapy zone one 1114 may comprise a therapy zone in which tissue within the target site being ablated predominantly by irreversible electroporation. Therapy zone two 1116 may comprise a therapy zone in which irreversible electroporation and temperature-related cell death mechanisms working together. The increase in normalized current 720 may be caused by a conductive rise due to an increase in the base target tissue temperature. In general, there is −2% rise in conductivity of tissue for each degree rise in temperature of ablated tissue. The temperature rise may begin to occur in zone one but is dependent on extrinsic and intrinsic factors. Therapy zone three 1118 may comprise a therapy zone in which the tissue within the target site is no longer the predominantly impacted by the irreversible electroporation cell death mechanism due to the fact that most of the electrical conductivity changes in the tissue is resulted from temperature changes (i.e., predominantly thermal ablation and/or electrolysis) and not the IRE effects. With some examples, processor(s) 708 in executing instructions 716 can determine therapy zones and transitions between therapy zones based on the normalized current 720.

At block 1308 “predict future normalized current based on normalized current and ablation therapy protocol parameters” future normalized current can be predicted based on actual normalized current 720 and ablation therapy protocol parameters 724. For example, processor(s) 708 in executing instructions 716 can predict future normalized current for the ablation therapy given past normalized current 720 and the ablation therapy protocol parameters 724. With some examples, processor(s) 708 in executing instructions 716 can predict future normalized current based on machine learning models trained on completed ablation therapy protocols. As another example, processor(s) 708 in executing instructions 716 can predict future normalized current based a mathematical relationship between normalized current 720 and ablation therapy protocol parameters 724. Predicted future normalized current 1412 is depicted in plot 1400. In particular, plot 1400 depicts both actual normalized current 720 (e.g., normalized current for voltage pulses actually applied during treatment) and predicted future normalized current 1412 (e.g., normalized current for voltage pulses not yet applied but scheduled based on ablation therapy protocol parameters 724, or the like). Accordingly, plot 1400 provides an intra-treatment picture of where within the therapy zones the treatment therapy is and also where within the therapy zones the treatment therapy might progress based on actual protocol parameters 724.

At block 1310 “generate GUI element(s)” graphical user interface (GUI) elements can be generated. For example, in executing instructions 716 processor(s) 708 can generate graphical data (e.g., display frames, or the like) including indications of GUI element 1410a, 1410b, 1410c, 1410d, 1410e, and 1410f. With some examples, GUI elements can include an indication of where within the therapy zones the ablation treatment is, an indication that the ablation therapy treatment is approaching a transition between zones (e.g., GUI element 1410a, GUI element 1410d, or the like), a query if entering a transition zone and want to continue, a query if there is a parameter change (e.g., voltage, waveform, number of pulses), a query if there are any physical changes to probes (e.g., pull back length, reposition probes, change electrode exposure length), a query to continue the ablation therapy treatment, or said differently continue application of voltage pulses (e.g., GUI input element 1410g, GUI input element 1410h, GUI input element 1412, or the like).

At block 1312 “generate a graphical information element comprising an indication of a plot of the normalized current, determined treatment zones, predicted future normalized current, and optionally, GUI elements” graphical data (e.g., display frames, or the like) representing plot 1400 can be generated comprising indications of normalized current trend line 1402, predicted future normalized current 1412, treatment zone one 1114, treatment zone two 1116, treatment zone three 1118, GUI elements 1410a, to 1410h. The graphic data can be stored in memory 714 as graphical information element 726.

At block 1314 “send the graphical information element to a display device to display the plot” the ablation therapy device can send the graphical information element 726 to display 710 to display the plot indicated by the graphical information element 726. For example, in executing instructions 716 processor(s) 708 can send the graphical information element 726 to display 710 and display 710 can display the plot indicated by the graphical information element 726.

In some examples, routine 1300 can be repeated such that display 710 can be updated with indications of plot 1400 with updated information (e.g., updated normalized current trend line 1402, updated predicted future normalized current 1412, updated GUI elements, or the like) as the treatment progresses.

Further, routine 1300 can be repeated individually for each pair of probes or collectively for all probe pairs. For example, routine 1300 can be implemented such that a plot depicting normalized current from one pair of probes can be generated at block 1308 and can be repeated such that another plot depicting normalized current from another pair of probes can be generated at block 1308. In some examples, both plots can be displayed on display 710. In other examples, a single plot depicting normalized current from multiple pairs of probes can be generated.

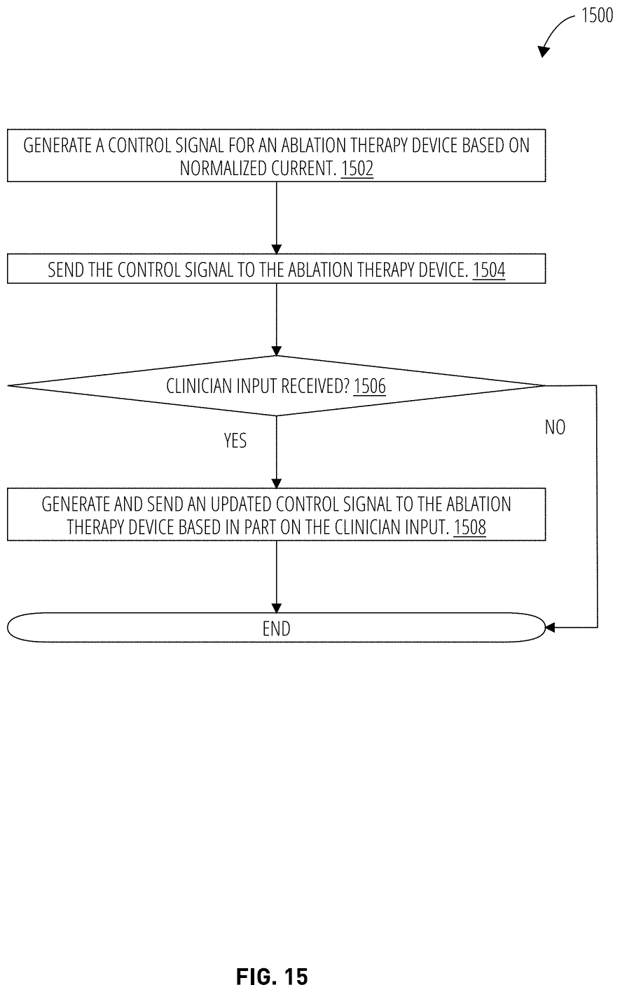

FIG. 15 depicts a routine 1500 that may be implemented by an ablation therapy device according to examples of the present disclosure. In some examples, routine 1500 can be implemented to control the ablation therapy device based on normalized current. At block 1502 “generate a control signal for an ablation therapy device based on normalized current” control signal 722 for ablation therapy device 700 can be generated based on normalized current 720. For example, processor(s) 708 in executing instructions 716 can generate control signal 722. As a specific example, where normalized current 720 indicates that the ablation therapy treatment is at a transitions between treatment zones (e.g., at transition 1110 as indicated by plot 1400 of FIG. 14, or the like), processor(s) 708 in executing instructions 716 can generate a control signal including an indication for voltage generator 704 to pause generation of voltage pulses. As another example where the normalized current 720 increases more than a threshold value (e.g., more than a percentage increase, more than a magnitude of increase, or the like), processor(s) 708 in executing instructions 716 can generate a control signal including an indication for voltage generator 704 to pause generation of voltage pulses.

At block 1504 “send the control signal to the ablation therapy device” control signal 722 can be sent to the ablation therapy device 700. For example, controller 106 can send the control signal 722 to voltage source 104. At decision block 1506 “clinician input received?” a determination can be made whether clinician input 728 is received. For example, responsive to a GUI input element (e.g., GUI element 1410a, etc.), processor(s) 708 in executing instructions 716 can receive input from a clinician. As a specific example, a clinician can use I/O devices 712 to provide a response to the GUI element and/or GUI input element and processor(s) 708 can receive the response at decision block 1506. From decision block 1506, routine 1500 can continue to either block 1508 or can end. For example, routine 1500 can proceed from decision block 1506 to block 1508 based on a determination that clinician input was received while routine 1500 can end based on a determination that clinician input was not received.

At block 1508 “generate and send an updated control signal to the ablation therapy device based in part on the clinician input” an updated control signal can be generated and sent to the ablation therapy device based on the clinician input 728. For example, where the clinician input is to continue the ablation therapy treatment, the updated control signal 722 can include an indication to resume generating and applying voltage pulses to the target tissue 112. In other examples, the clinician input can be an indication to change protocol parameters 724 (e.g., change voltage, change number of pulses per round, or the like).

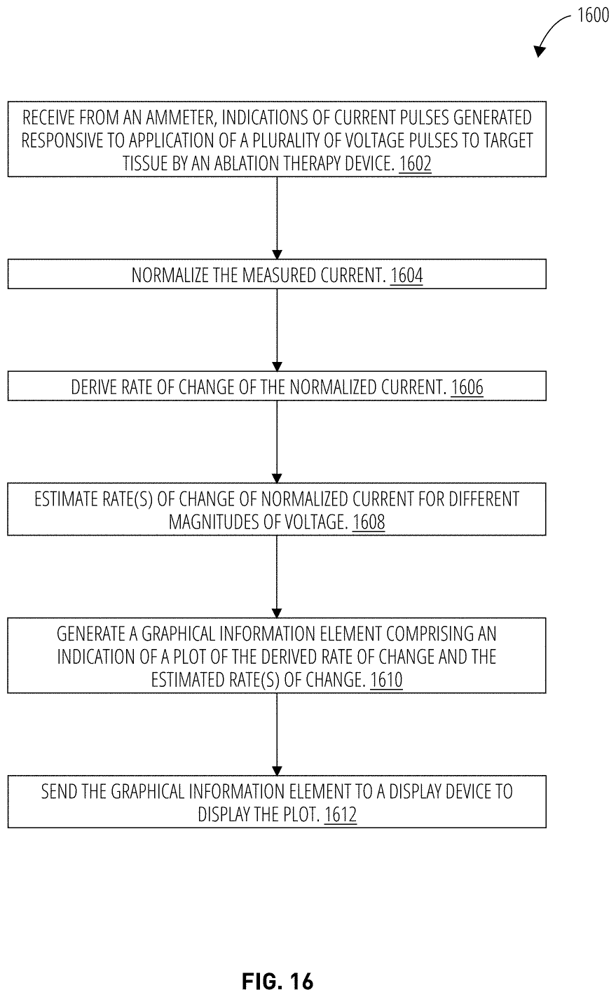

FIG. 16 depicts a routine 1600 that may be implemented by an ablation therapy device according to examples of the present disclosure. The routine 1600 of FIG. 16 and plots of FIG. 36A to FIG. 36D are described together herein. In some examples, routine 1600 can be implemented to generate graphical data for display on display 710. FIG. 36A to FIG. 36D depicts example plots that may be generated based on routine 1600. In particular, FIG. 36A to FIG. 36D shows plots depicting rate of change of normalized current and estimated rate of change of normalized current for a probe pair over a number of voltage pulses of an ablation therapy treatment.

The rate of change of normalized current can be used to indicate an “intensity” of an IRE or H-FIRE procedure. The intensity can be defined in the first derivative of the normalized current as a constant value either positive, zero, or negative. When the intensity is positive, IRE intensity is higher and/or has a stronger effect on the target tissue. In the normalized current graph as shown in FIG. 14, if the slope of real-time data of normalized current is positive, the IRE intensity is stronger as compared with the slope of real-time data of normalized current is negative (as seen during the transition 1110 and 1112). Therefore, when the real-time data of normalized current has a steeper slope (ex: between pulses 3-20) the IRE has a stronger intensity on target tissue as compared to when the real-time data of normalized current has a shallow slope (ex: between pulses 21-125). In other words, the intensity of the IRE and/or H-FIRE procedure may include the completeness of irreversible electroporation of tissue within a target site, the type of cell death of the tissue, and/or the potential effects the ablation may have on an immune response by the body. In general, a more positive rate of change of the normalized current (e.g., positive first derivative of the normalized current) indicates a more intense IRE or H-FIRE procedure. The present disclosure can be implemented to determine an intensity of an ablation modality (e.g., IRE, H-FIRE, etc.) and compare the determined intensity with estimated or predicted intensities for the procedure at different voltages. With some examples, routine 1600 can generate graphical information element including indication of a plots 3604, 3606, 3608, and 3610 providing a graphical representation of the intensity of an IRE and/or H-FIRE procedure for various voltage settings. At block 1602 “receive from the ammeter, indications of current generated responsive to application of a plurality of voltage pulses to the target tissue by an ablation therapy device” indications of current measured at an ammeter can be received. For example, in executing instructions 716 processor(s) 708 can receive measured current 718 from ammeter 706.

At block 1604 “normalize the measured current” the measured current can be normalized. For example, in executing instructions 716 processor(s) 708 can normalize the measured current 718 to generated normalized current 720. In some examples, processor(s) 708 can execute instructions to normalize the measured current for voltage. Said differently, processor(s) 708 can execute instructions 716 to normalize measured current 718 to a common reference point, resulting in normalized current 720.

At block 1606 “derive rate of change of normalized current” a rate of change of normalized current 720 can be derived. For example, processor(s) 708 in executing instructions 716 can determine an actual rate of change of the normalized current 720 using Equation 5 described herein.

At block 1608 “estimate rate(s) of change of normalized current for different magnitudes of voltage” rates of change of normalized current can be estimated for different magnitudes of voltage with which an ablation therapy could be applied. For example, processor(s) 708 in executing instructions 716 can estimate a rate of change of normalized current for other ablation therapy procedure voltages (e.g., voltage magnitudes different than the current magnitude, or the like). For example, processor(s) 708, in executing instructions 716, can determine and display a plot comprising an estimated rate of change of normalized conductivity versus pulse numbers (not shown), and/or normalized conductivity versus voltage gradient (not shown).

At block 1610 “generate a graphical information element comprising an indication of a plot of the derived rate of change and the estimated rate(s) of change” graphical data (e.g., display frames, or the like) representing plots can be generated comprising indications of change in normalized current 3604, the rate of change of normalized current 3606, change in percentage of change in conductivity plot 3608, and indication of combined treatment data from multiple physicians plot 3610. The graphic data can be stored in memory 714 as graphical information element 726.

At block 1612 “send the graphical information element to a display device to display the plot” the ablation therapy device can send the graphical information element 726 to display 710 to display the plot indicated by the graphical information element 726. For example, in executing instructions 716 processor(s) 708 can send the graphical information element 726 to display 710 and display 710 can display the plot indicated by the graphical information element 726 (e.g., plots 3604, 3606, 3608, and 3610).

In some examples, routine 1600 can be repeated such that display 710 can be updated with indications of plots 3604, 3606, 3608, 3610 with updated information as the treatment progresses. Further, routine 1600 can be repeated individually for each pair of probes or collectively for all probe pairs. For example, routine 1600 can be implemented such that a plot depicting normalized current from one pair of probes can be generated at block 1608 and can be repeated such that another plot depicting normalized current from another pair of probes can be generated at block 1608. In some examples, both plots can be displayed on display 710. In other examples, a single plot depicting actual rate of change from multiple pairs of electrodes (not shown) can be generated.

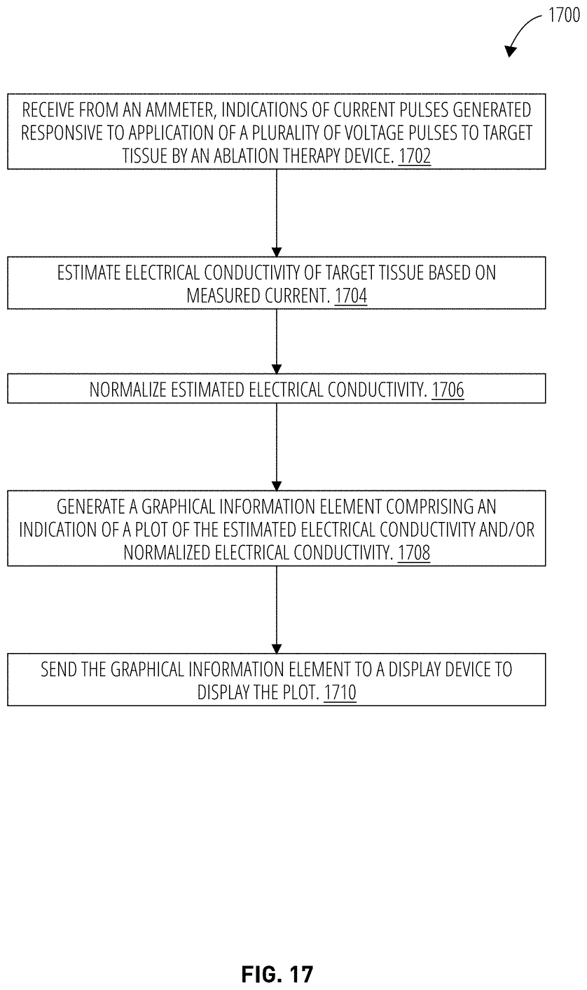

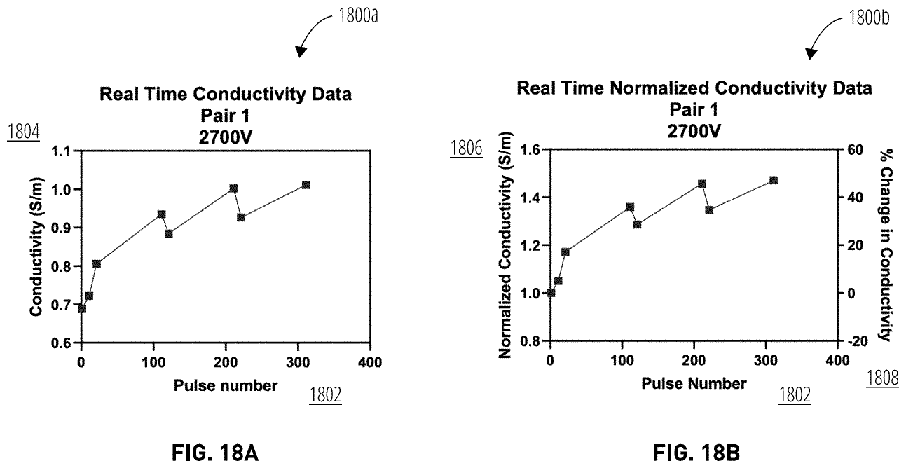

FIG. 17 depicts a routine 1700 that may be implemented by an ablation therapy device according to examples of the present disclosure. In some examples, routine 1700 can be implemented to generate graphical data for display on display 710. For example, FIG. 18A and FIG. 18B depict examples of plots 1800a and 1800b, respectively, which may be generated on routine 1700. The routine 1700 of FIG. 17, plot 1800a of FIG. 18A, and plot 1800b of FIG. 18B are described together herein.

At block 1702 “receive from the ammeter, indications of current pulses generated responsive to application of a plurality of voltage pulses to the target tissue by an ablation therapy device” indications of current measured at an ammeter can be received. For example, in executing instructions 716 processor(s) 708 can receive measured current 718 from ammeter 706.

At block 1704 “estimate electrical conductivity of target tissue based on measured current” electrical conductivity of target tissue can be estimated (e.g., derived, or the like) based on measured current 718. For example, processor(s) 708 in executing instructions 716 can estimate electrical conductivity of target tissue 112 given measured current 718. It is to be appreciated that electrical conductivity and measured current 718 have close relationship and that normalized values of both are substantially the same if the shape factor is assumed to be constant throughout the ablation therapy procedure. Equation 1 and 2 detailed above illustrate the relationship between current (I), shape factor (S), and electrical conductivity (a). The shape factor, S, defines probe-specific characteristics which impact tissue response and the resulting current measurement. Probe characteristics include probe dimensions, electrode dimensions and distance between probes. As an example, the following Equation 6 can be used to represent probe dimensional when using two or more cylindrical probes placed in a parallel relationship in the target tissue. Where S=shape factor, L=electrode exposure length; D1=diameter of probe #1, D2=diameter of probe #2; z=distance between probe #1 and probe #2. Note, it is within the conception of this disclosure to use more than two probes, and Equation 6 would be adjusted to reflect a total probe count.

$$S = \frac{2 π L}{{cosh}^{- 1} ( \frac{4 {z}^{2} - {D}_{1}^{2} - {D}_{2}^{2}}{2 {D}_{1} {D}_{2}} )} \qquad EQUATION 6$$

At block 1706 “normalize estimated electrical conductivity” the estimated electrical conductivity can be normalized. For example, processor(s) 708 in executing instructions 716 can normalize the estimated electrical conductivity derived at block 1704 to a common reference point. Said differently, processor(s) 708 in executing instructions 716 can normalize estimated electrical conductivity for applied voltage.

At block 1708 “generate a graphical information element comprising an indication of a plot of the estimated electrical conductivity and/or the normalized estimated electrical conductivity” a graphical data (e.g., display frames, or the like) representing plot 1800a and/or plot 1800b can be generated. For example, processor(s) 708 in executing instructions 716 can generate graphical information element 726 comprising an indication of plot 1800a where estimated electrical conductivity 1804 is depicted on the Y axis and voltage pulses 1802 on the X axis. Alternatively, or additionally, processor(s) 708 in executing instructions 716 can generate graphical information element 726 comprising an indication of plot 1800b where normalized electrical conductivity 1806 is depicted on the Y axis and voltage pulses 1802 on the X axis. Further, as depicted in plot 1800b, the change in normalized electrical conductivity 1808 is depicted on the Y axis. The graphic data can be stored in memory 714 as graphical information element 726.

At block 1710 “send the graphical information element to a display device to display the plot” the ablation therapy device can send the graphical information element 726 to display 710 to display the plot indicated by the graphical information element 726. For example, in executing instructions 716 processor(s) 708 can send the graphical information element 726 to display 710 and display 710 can display the plot indicated by the graphical information element 726 (e.g., plot 1800a, plot 1800b, or the like).

In some examples, routine 1700 can be repeated such that display 710 can be updated with indications of updated plot 1800a, updated plot 1800b to provide intra-treatment indications of estimated electrical conductivity and/or normalized electrical conductivity as the treatment progresses. Further, routine 1700 can be repeated individually for each pair of probes or collectively for all probe pairs.

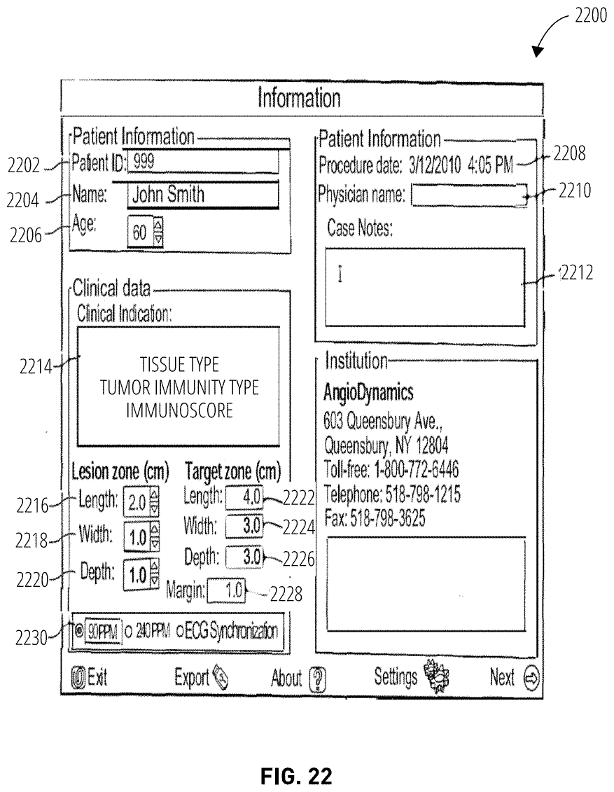

FIG. 19 illustrates a plot 1900 depicting the use of normalized current data to identify the onset of pore formation for reversible electroporation in zone zero and the transition to predominantly irreversible electroporation in zone one. In one example, zone zero represents the onset of pore formation for reversible electroporation that will occur within the first few pulses (i.e., the first 3-5 pulses). As the rate of normalized current increases after 3-5 pulses, zone zero 1904 (predominantly RE) transitions 2202 to zone one 1114 (predominantly IRE and/or HFIRE) which in this example may extend for at least 120 pulses. As show in FIG. 22, the slope of normalized current changes in steepness at around 20-21 pulses, with pulses 1-20 having a steeper slope for normalized current as compared with pulses deliver after pulse 21. This change in steepness in slope of normalized current is a result of the end of a round of pulses. After each round of pulses delivered to the target tissue, the conductivity (and reversibility of the pores in the cellular membrane) of the target tissue is changed. In one example, and as shown in FIG. 19, the first round of pulses ended at around pulse 20 and the second round of pulses started at around pulse 21. This change in round of pulses can be seen in the change in steepness of the slope of normalized current.

Banks of tissue-specific treatments from previously documented reversible electroporation procedures may also be used to optimize individual reversible electroporation treatment protocols. In addition to normalized current data, the bank may contain information on pore characteristics of specific cell types, including electrical field thresholds required to achieve onset of pore formation, the maximum electrical threshold before the cell type is unable to recover and pore size at a particular point in the procedure. The database may also include lookup tables on specific chemical species being introduced into the cell including but not limited to macromolecule type, size, and recommended pore size. As discussed above, the GUI interface may be used by the clinician to input treatment parameters specific to reversible electroporation such as macromolecule and target tissue cell type. Based on the clinician input and information from the databank, the therapy device may display recommended treatment parameters including recommended number of pulses to achieve optimal uptake by the cell while still maintaining cell viability. A benefit of the bank of tissue-specific treatment data includes, but is not limited to, predicting the current response after an increase or decrease in the applied voltage before or during a procedure and/or avoiding overcurrent. Clinicians may apply different voltages as treatment planning. They also may change the voltage during the procedure, which affect the current response. Therefore, by using a bank of tissue (of course normalized), you can predict the nature of current trend after the changed voltage point. This is important not only to predict the current trend after the changed voltage but also to avoid misunderstanding of current rise due to the ohmic effect and not IRE. Furthermore, by using the bank of normalized current for variety of tissue at different voltages, one will be able to predict the current trend, specially at the critical voltages where the chance of arcing is higher.

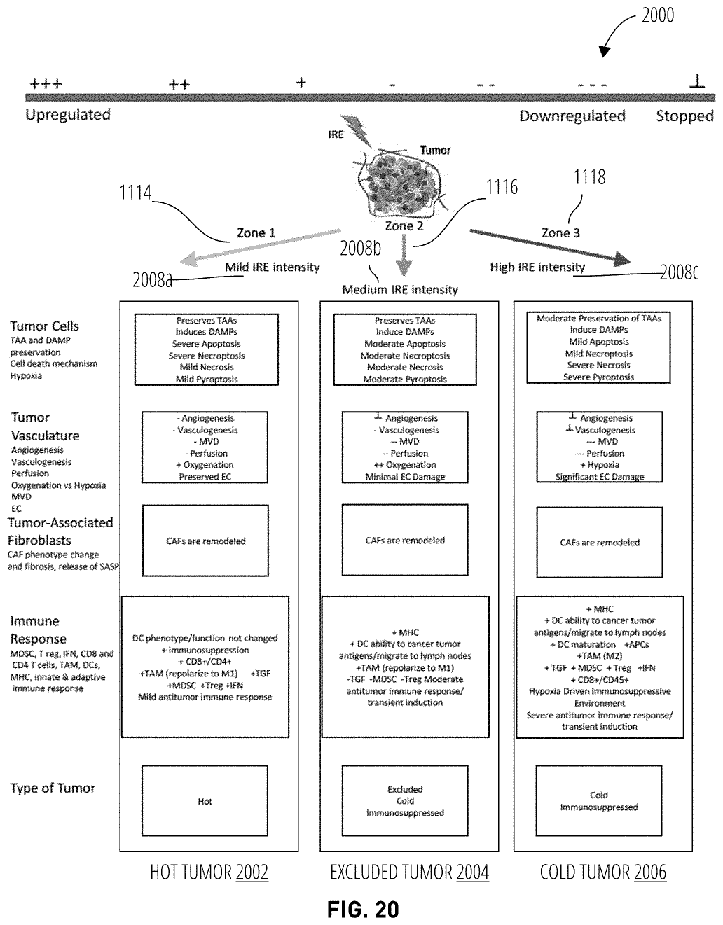

As described above, the present disclosure can be implemented to generate a recommended treatment plan including a treatment intensity based on clinician provided characteristics or data (e.g., clinician input 728, or the like), such as, an immunity type of tumor to be treated and/or an immunoscore of a tumor to be treated. FIG. 20 illustrates a chart 2000 showing the effects of various doses of IRE or H-FIRE on the component of tumor microenvironments. IRE and H-FIRE doses effect cancer cells and the surrounding tumor microenvironment differently, including tumor vascularization, immune system cells, and CAFs. More specifically, chart 2000 depicts effects of IRE and/or H-FIRE treatment on different types of tumors and at various intensity levels.

For example, in zone one 1114, only IRE with no temperature affect is applied at a first intensity level 2008a to a hot tumor 2002. As can be seen, the vasculature structure is preserved such that the blood supply is good and so is oxygenation. The angiogenesis and vasculogenesis decreased due to the relative remodeling in vascular structure. The extent of apoptotic response would induce anti-inflammatory cell death in cancerous cells while preserving the extracellular membrane structure intact. The lack of competing cancerous cells would provide enough oxygen for infiltrated coming T cells. The cancer cells are mostly killed, the TAAs are preserved, and the immature DCs (besides recognizing the TAAs) need oxygen to get to the mature population and initiate the systemic response. If CD4 and CD8 T cells can be infiltrated without presence of MDSC or T regs, the oxygen level would be enough to support the already activated adaptive response. In some embodiments, IRE of a “mild intensity” or at a lower intensity level with respect to other available intensity levels is recommended for hot immunoscore tumor types, or hot tumors 2002. In hot immunoscore, the innate and immune responses are recognizing the cancer cells and enough T cells are infiltrated into the tumor microenvironment. Accordingly, by usage of zone one 1114 IRE for hot tumor types, the effect of primary adjuvant therapies in addition to positive changes to CAFs to support anti-tumor response may be intensified.

In zone two 1116, the angiogenesis has stopped with good control of the vasculogenesis. Therefore, the abnormal perfusion rate is decreased and since there are less competing cancerous cells the immune system can function better. This treatment zone may correspond to a second intensity level 2008b, or rather a medium intensity or intensity level in the mid point of available intensity levels and may be applied to excluded tumors 2004, such as excluded or immunosuppressive tumors. Treatments in zone two 1116 can invoke a combination of anti-inflammatory and proinflammatory cell death mechanisms where close to the electrodes and high intensity electrical fields, the other ablation modalities dependent damage will contribute to necrosis and pyroptosis, however as you go further from the electrodes, the apoptotic and necroptotic cell death mechanisms become more dominant. The whole tumor microenvironment is going through nonhomogeneous cell death mechanisms from center to periphery. The regions of zone two 1116 that are undergoing apoptosis and necroptosis probably contain TAAs and therefore are easier to be recognized by immature DCs whereas the rest of zone two 1116 is undergoing severe damage from other ablation modalities which may stop angiogenesis and hence provide more oxygen for the coming infiltrated T cells. Since the extent of IRE on the boundary of the tumor will remodel the CAFs, the chance of T cell infiltration in the excluded immunoscore tumors is increased.

In zone three 1118, IRE with strong effect from other ablation modalities. This treatment zone may provide a similar immune response to using traditional ablation therapies in addition to IRE where the traditional ablation therapies treat the core of the tumor and IRE effect trims the edges. In some embodiments, treatments of a high intensity, or intensity level 2008c, are recommended for cold tumors 2006. In this treatment zone, angiogenesis and vasculogenesis stops because of the severe damage to the cancerous tissue and so the perfusion rate drops significantly. However, since the supply of oxygen depends on the number of vasculature and perfusion rate, the tissue undergoes hypoxia. Initially, the severe necrosis and pyroptosis cell death mechanisms induced by aggregated effect of the other ablation modalities of intensified IRE causes angiocongestion and vasculogenesis is paused which severely damages the microvascular density and causes very low blood perfusion which causes both ischemia and hypoxia. The impact of zone three 1118 on an extracellular membrane is significant and due to the plasma membrane disruption, the proinflammatory signals are activated and so the innate and adaptive immune responses. This scenario would serve a case where the adaptive immune system is not yet activated, and therefore, there is a need to induce this systemic immune response (cold tumors, immunosuppressed). It is to be appreciated, that after a few hours to days, the angiogenesis and vasculogenesis will happen as a natural healing process due to the injury, and therefore there will be enough oxygen supply for the infiltrated cells which can be provided by other adjuvant therapies to the tumor microenvironment.

Tumor Associated Fibroblasts (CAFs) play an important role in controlling the tumor microenvironment and tumor growth. They control the extracellular membrane remodeling and release different cytokines and chemokines, and are also affecting the checkpoint inhibitors which may ultimately decide how the tumor is categorized. IRE technology can preserve the extracellular membrane while affecting the hemostasis of the critical structure. In all zones of IRE delivery, these tumor associated fibroblasts, which are located at the border of the tumor mass, are remodeled but only affected by IRE with no damage from other ablation modalities.

As described in this disclosure, ablation modalities or ablation therapies may include, but are not limited to, IRE ablation, H-FIRE ablation, radio frequency (RF) ablation, microwave ablation, cryo ablation, a rise in temperature, pulse-field ablation, or a thermal ablation modality.

Zone two 1116 and zone three 1118 may be interchangeable for cold and immunosuppressed tumors depending on the initial goals set by physician for treatment planning strategies. Due to more significant other ablation modality effects of zone three 1118, stronger TAAs and TSAs presentat, while the lack of oxygen and immunosuppression affinity may affect the infiltration success. In zone two 1116, the presence of TAAs and TSAs are less when compared to zone three 1118, however less immunosuppression and enough oxygenation is expected. Methods and systems are provided herein to design the right choice of IRE and/or H-FIRE zones by considering the detailed role and dosage of other adjuvant/neoadjuvant therapies.

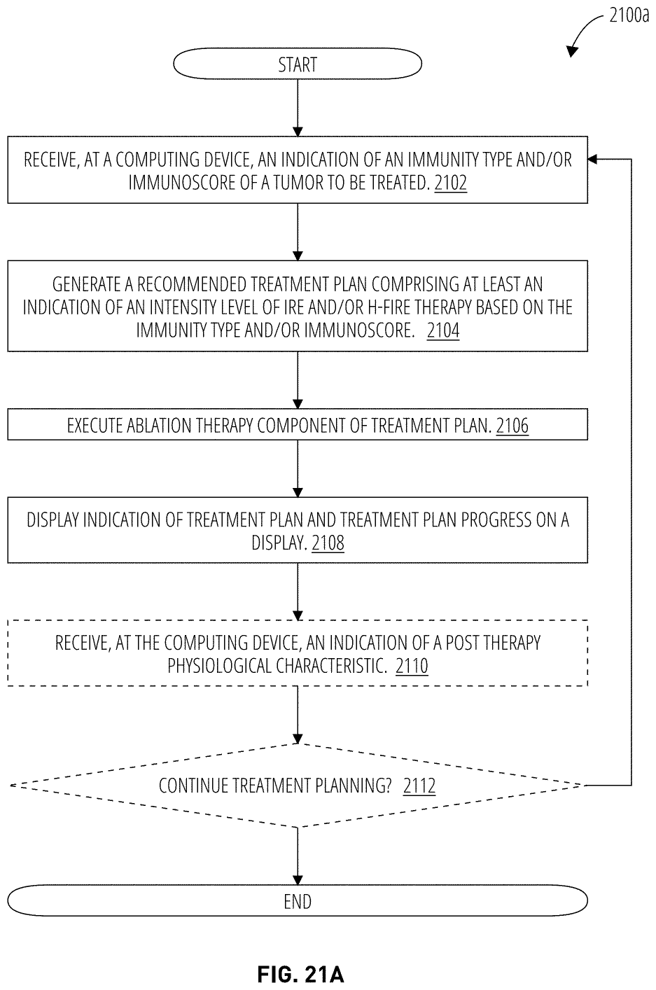

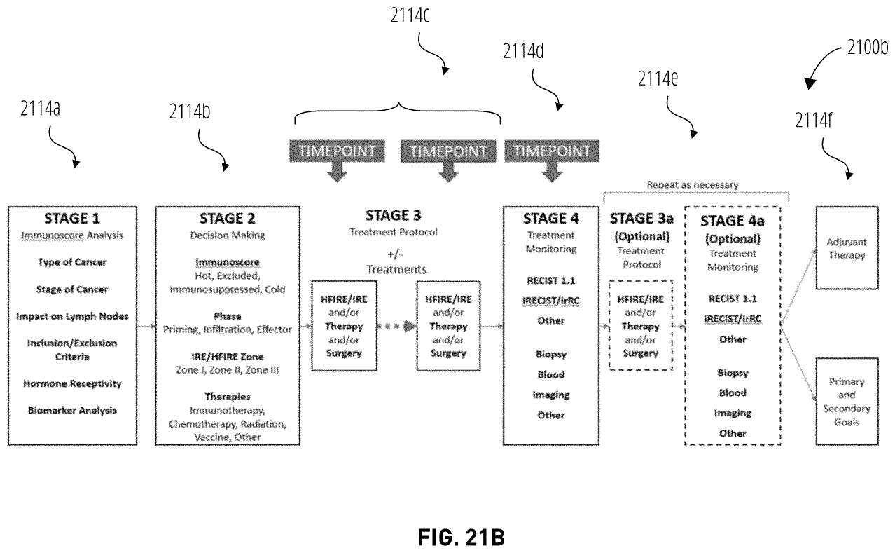

FIG. 21A illustrates a routine 2100a, according to some embodiments of the present disclosure. Routine 2100a can be implemented by an IRE and/or H-FIRE device, such as, for example, ablation therapy system 100 or ablation therapy device 700. Routine 2100a can begin at block 2102. At block 2102 “receive, at a computing device, an indication of an immunity type and/or immunoscore of a tumor to be treated” an indication of an immunoscore of a patient can be received. For example, in executing instructions 716, processor(s) 708 can receive clinician input 728 comprising indications of an immunoscore of target tissue 112 or an immunoscore of a tumor to be treated as contemplated herein. With some examples, processor(s) 708 can execute instructions 716 to generate a graphical user interface to receive such indications and also to display such indications to a user. For example, FIG. 22 illustrates a GUI 2200 comprising text input boxes to receive indications of patient ID 2202, patient name 2204, patient age 2206, procedure date 2208, physician name 2210, and case notes 2212. Additionally, GUI 2200 comprising input boxes to receive indications of clinical data 2214 (e.g., tissue type, tumor immunity type such as, hot, immunosuppressed, excluded, cold, or the like, and an immunoscore) as well lesion and target zone information related to the target tissues 112 (e.g., lesion length 2216, 2218, lesion depth 2220, target zone length 2222, target zone width 2224, target zone depth 2226 and margin 2228). Additionally, GUI 2200 can comprise indications of a recommended IRE and/or H-FIRE procedure, such as pulses per minute 2230.

Returning to FIG. 21A and routine 2100a, with some examples, at block 2102, processor(s) 708 can execute instructions 716 to receive an indication of an immunoscore of a tumor and determine the tumor immunity (e.g., hot, immunosuppressed, excluded, cold, etc.) based on the immunoscore. As another example, at block 2102, processor(s) 708 can execute instructions 716 to receive an indication of a tumor immunity type and/or immunoscore and one or more other treatment characteristics, such as, tissue type, cancer type, cancer stage, impact of lymph nodes, inclusion and exclusion criteria, hormone receptivity, biomarker analysis, treatment phase (e.g., priming, infiltration, effector, or the like), or conjunctive therapies (e.g., immunotherapy, chemotherapy, radiation, vaccine, etc.).

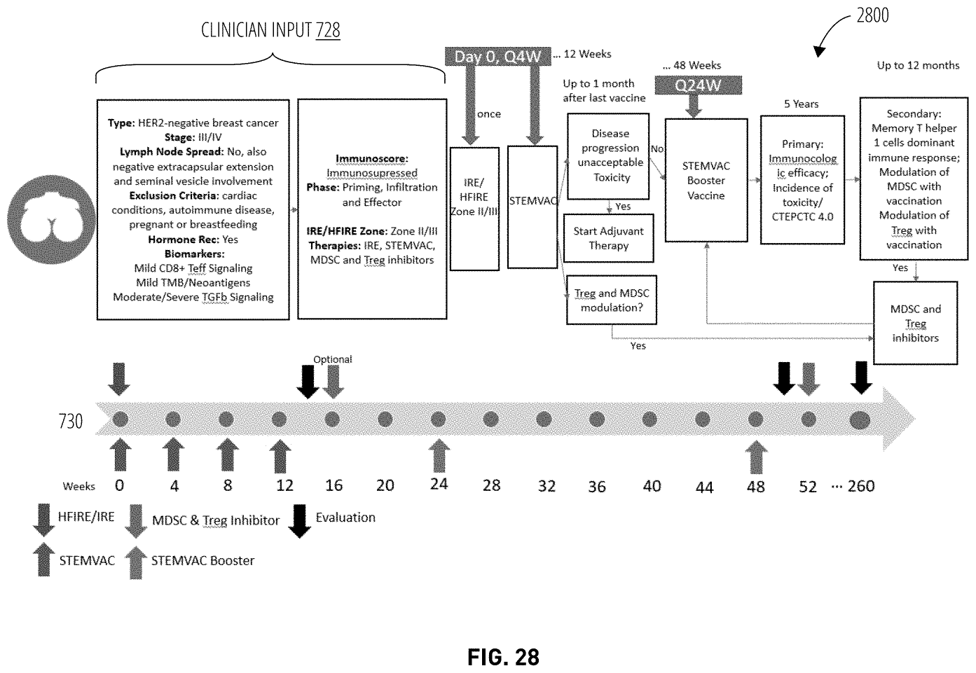

Routine 2100a can continue to block 2104 “generate a recommended treatment plan comprising at least an indication of an intensity level of IRE and/or H-FIRE therapy based on the immunity type and/or immunoscore” a recommended treatment plan can be generated based on the immunoscore and/or immunity type. For example, processor(s) 708 can execute instructions 716 to generate treatment plan 730. In general, treatment plan 730 can comprise an indication of a treatment zone or a treatment intensity. As a specific example, processor(s) 708 can execute instructions 716 to generate an indication of one of a plurality of predefined intensity levels, or zones, such as, (e.g., zone one 1114, zone two 1116, zone three 1118, etc.) and IRE and/or H-FIRE protocol parameters 724 associated with the recommended zone. With some examples, the treatment plan 730 can comprise indications of a timing of application of IRE and/or H-FIRE relative to other conjunctive therapies outlined in the treatment plan (e.g., relative to immunotherapy, chemotherapy, radiation, vaccine, or the like). With some examples, a machine learning model can be trained to generate or infer the treatment plan 730. This is described in greater detail below. Further, as will be apparent from this disclosure, the treatment plan 730 can include an indication of protocol parameters for an IRE and/or H-FIRE procedure to provoke or provide a recommended level of intensity of IRE and/or H-FIRE therapy. Additionally, in some examples, the treatment plan 730 can include indications of a timing of the IRE and/or H-FIRE therapy relative to other adjunctive (or complementary) therapies and can even include recommendations of other adjunctive therapies. For example, FIG. 28 to FIG. 31 illustrate diagrams representative of treatment plans that can be generated according to the present disclosure. As will be appreciated, these treatment plans comprise both an IRE and/or H-FIRE intensity component and adjunctive treatment components as well as prescribed timing for the various components of the treatment plan 730. It is noted that the devices described herein are often not capable of implementing the recommended complementary (or adjunctive) therapy. However, the treatment plan 730 could still include indications about the adjunctive therapy.

With some examples, the treatment plan 730 and/or protocol parameters 724 can include an indication of the predefined intensity level, a recommended voltage, a pulse length, a number of pulses to deliver, or the like. As a specific example, in some embodiments, processor(s) 708 can execute instructions 716 to receive an indication of a placement of probes (e.g., FIG. 5A and FIG. 5B) as well as an indication of a distance between the probes. Responsive to receiving an indication of probes and the distance between the probes and immunity type and/or immunoscore, the processor(s) 708 can execute the instructions 716 to generate the treatment plan 730 including protocol parameters 724, which can include an indication of a voltage to be applied (e.g., 2100V, 2400V, 2700V, 3000V, or the like) as well as an intensity level or treatment zone. In some embodiments, the intensity level can include an associated normalized current to indicate an “end” of the zone. With some embodiments, processor(s) 708 can execute instructions 716 to generate protocol parameters 724 where the volts per centimeter is approximately 1500 (e.g., voltage for a probe pair is based on the distance between to the probes).

With some examples, the processor(s) 708 can execute instructions 716 to generate treatment plan 730 and/or protocol parameters with an intensity level and associated normalized current as outlined in Table 4 below.

TABLE 4

| Voltage | Zone 1 | Zone 2 | Zone 3 |
|---|---|---|---|
| 2100 | 1.20-1.30 | 1.25-1.35 | 1.25-1.35 |
| 2400 | 1.25-1.35 | 1.30-1.40 | 1.30-1.40 |
| 2700 | 1.30-1.40 | 1.35-1.45 | 1.35-1.45 |
| 3000 | 1.35-1.45 | 1.40-1.50 | 1.40-1.50 |

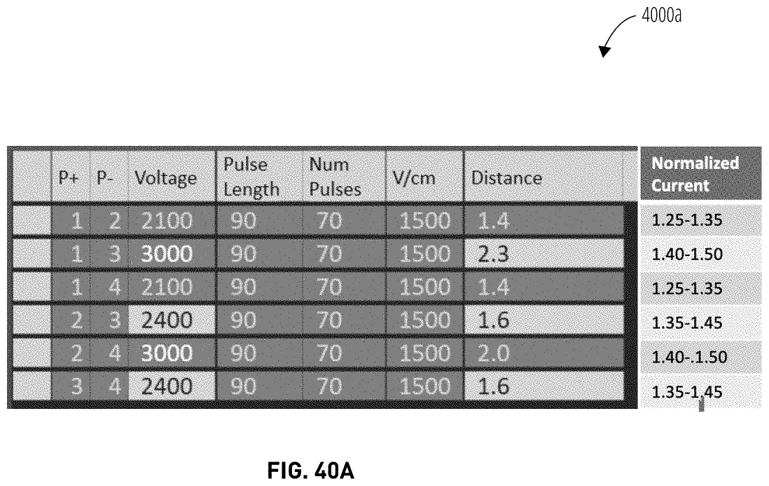

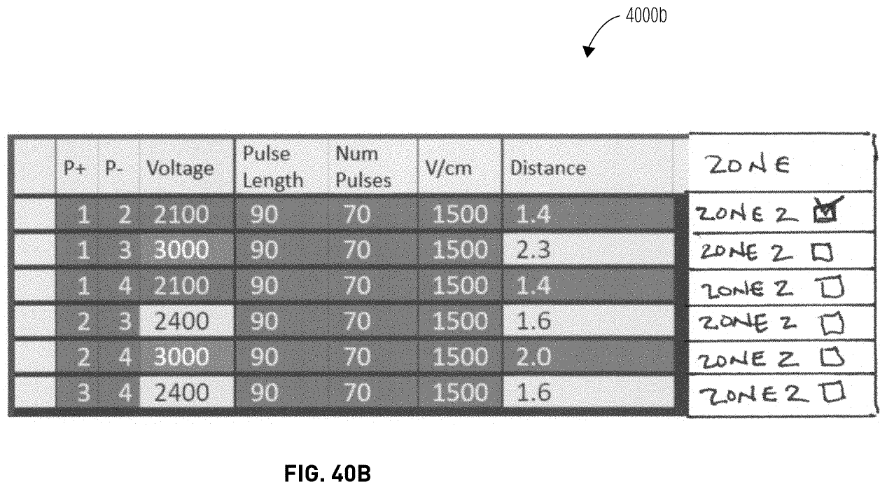

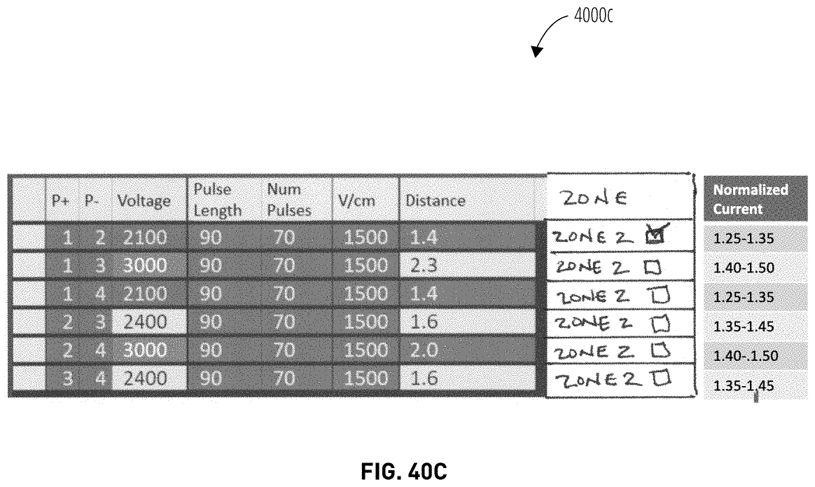

Continuing to block 2106 “execute ablation therapy component of treatment plan” the ablation therapy component of the treatment plan can be executed. For example, processor(s) 708 can execute instructions 716 to generate a graphical information element, such as, the graphical information element shown in FIG. 40A, 40B, or 40C. In general, these figures illustrate graphical information elements 4000a, 4000b, and 4000c, respectively, which depict indications of suggested protocol parameters 724 (e.g., polarity, voltage, pulse length, number of pulses, etc.) for each probe and probe pair. The graphical information elements can also include an indication of an intensity level (e.g., zone one 1114, zone two 1116, zone three 1118, etc.), an indication of the normalized current for the zone, or both. Further, the graphical information elements can include an indication of whether the intensity level has been achieved (e.g., via a check box as shown, via different color scheme, or the like). With some embodiments, the graphical information element 4000 can be generated as graphical information element 726 and displayed on display 710.

Further, processor(s) 708 can execute instructions 716 to receive an input from a user (e.g., physician, or the like) comprising an indication to accept the suggested or generated treatment plan 730 and protocol parameters 724. Responsive to the input, processor(s) 708 can execute instructions 716 to send control signals to voltage generator 704 to cause pulses to be applied to target tissue via probes as described herein using the protocol parameters 724 generated at block 2106. Further, processor(s) 708 can execute instructions 716 to determine a whether the normalized current (e.g., associated with the predefined intensity level) for the treatment plan 730 and protocol parameters 724 has been reached. For example, routine 2100a can implement, at block 2106, one or more routines described herein to monitor and/or determine the normalized current. Responsive to determining that the normalized current for one or each probe pair has been reached, processor(s) 708 can execute instructions 716 to generate a graphical information element comprising an indication that the predefined intensity has been applied (e.g., the treatment zone has been entered or exited, etc.) as described herein.

Continuing to block 2108 “display indication of treatment plan and treatment plan progress on a display” indication(s) of the treatment plan and/or the treatment plan progress can be displayed. For example, processor(s) 708 can execute instructions 716 to generate graphical information element 726 comprising indications of plots of treatment intensities of zones (e.g., plot 1100, or the like) and cause the graphical information element 726 to be displayed on display 710.

Routine 2100a can optionally include block 2110. For example, in some embodiments, the ablation therapy system 100 and/or ablation therapy device 700 can be arranged to monitor and/or receive indications about results of effects of an ablation procedure and generate an updated treatment plan based on the results. For example, routine 2100a can include block 2110 “receive, at the computing device, an indication of a post therapy physiological characteristic” a post-IRE and/or H-FIRE therapy physiological characteristic can be received. For example, processor(s) 708 can execute instructions 716 to receive indications of a response evaluation criteria in solid tumors (RECIST), immune-RECIST (iRECIST), or immune related response criteria (irRC). Furthermore, at block 2108, processor(s) 708 can execute instructions 716 to receive indications of results from a biopsy, a blood test, medical imaging (e.g., X-ray, ultrasound, CAT scan, MRI, or the like).

Routine 2100a can continue from block 2110 to decision block 2112. At decision block 2112 “continue treatment planning?” a determination of whether to continue treatment planning can be made. For example, processor(s) 708 can execute instructions 716 to determine whether to continue treatment planning, or rather, whether to generate a new treatment plan based on the immunity type and/or immunoscore and the physiological characteristic. For example, processor(s) 708 can execute instructions 716 to determine that given the immunity type and the physiological response another treatment plan 730 can be generated. In such an example, routine 2100a can return to block 2104 based on a determination that treatment planning should continue and can end based on a determination that treatment planning should not continue.

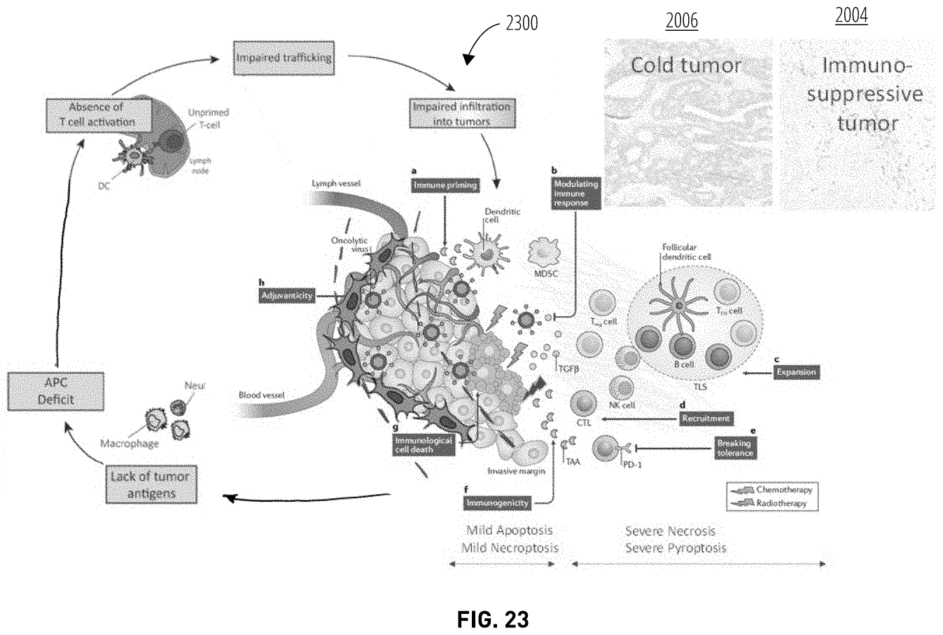

For example, in some embodiments, a treatment plan 730 can be generated for a particular immunity type tumor (e.g., a cold tumor) with the aim to “reverse” the tumor into another immunity type (e.g., a hot tumor). FIG. 23 illustrates a flow diagram 2300 depicting reversing a cold tumor 2006 into a hot tumor 2002. As can be seen, the absence of T cells in the tumor may be due to the lack of tumor antigens, APC deficit, absence of T cell priming/activation and impaired trafficking of T cells to the tumor mass. The present disclosure provides to generate a treatment plan 730 (protocol parameters 724) adapted to the step of the anti-cancer immune response that is not functional.

For example, for a cold tumor 2006, a treatment plan 730 comprising a treatment intensity level 2008c associated with a zone three 1118 can be generated for a cold tumor 2006 with the aim to reverse the cold tumor 2006 into a hot tumor 2002. As a result of application of the generated therapy, angiogenesis and vasculogenesis may stop because of the severe damage to the cancerous tissue. As such, the perfusion rate may drop significantly. However, since the supply of oxygen depends on the number of available vasculature system and perfusion rate, the tissue undergoes hypoxia. Initially, severe necrosis and pyroptosis cell death mechanisms induced by aggregated effect by other ablation modalities of intensified IRE causes angiogenesis and vasculogenesis to pause. These other ablation modality effects may damage the microvascular density severely and reduce the blood circulation which causes both ischemia and hypoxia.

It is noted that routine 2100a can be envisioned or implemented as a treatment workflow comprising several “stages” or steps. For example, FIG. 21B illustrates a workflow 2100b comprising stages 2114a, 2114b, 2114c, 2114d, and 2114e resulting in an ablation therapy (e.g., stage 2114c, or the like) as well as an adjunctive therapy (e.g., stage 2114f, or the like). The impact of an IRE and/or H-FIRE treatment applied with an intensity in zone three 1118 on an extracellular membrane is significant and due to the plasma membrane disruption, the proinflammatory signals are activated and so the innate and adaptive immune responses. This scenario would serve a case where the adaptive immune system is not yet activated. As such, the present disclosure provides to generate a treatment plan 730 to induce this systemic immune response for a cold tumor 2006. After a few days to weeks, the angiogenesis and vasculogenesis will happen as a natural healing process due to the injury, and therefore there will be enough oxygen supply for the infiltrated T lymphocyte cells which can be provided by other adjuvant therapies to the tumor microenvironment.

As another example, a treatment plan 730 can be generated to cause an immune response in another type of tumor (e.g., excluded tumor 2004, or the like). For example, processor(s) 708 can execute instructions 716 to generate protocol parameters 724 associated with a zone two 1116 IRE and/or H-FIRE treatment, which when applied to target tissue 112 to induce innate immune response in the tissue similar to what is described above for the cold tumor 2006. It is noted that treatment plans 730 can be generated for zone two 1116 or zone three 1118 for reversing cold tumors 2006 or excluded tumors 2004 into hot tumors 2002. Due to more significant ablation effects of zone three 1118, it is expected to see a stronger TAAs and TSAs presentation, while the lack of oxygen and immunosuppression affinity may affect the infiltration success. Whereas in zone two 1116, the presence of TAAs and TSAs are less compared to zone three 1118, however less immunosuppression and enough oxygenation is expected. The treatment plan generated by the ablation therapy system 100 and/or ablation therapy device 700 can be generated based on inputs received from a physician to recommend an IRE and/or H-FIRE treatment to promote an immune response in the tumor. Once this immune response is triggered, adjunctive therapies can be applied, such as, for example those described above.

It is noted, that with some examples, a first treatment plan 730 can be generated and applied (e.g., a zone two 1116 treatment plan and based on feedback from post therapy physiological responses (e.g., block 2108 of routine 2100, or the like) an additional treatment plan 730, or a continuation of the prior treatment plan 730 can be generated. For example, a cold tumor 2006 may be reversed into an excluded tumor 2004 based on application of a zone two 1116 treatment and the excluded tumor 2004 can be further reversed into a hot tumor by continuing the zone two 1116 treatment into a zone three 1118 treatment.

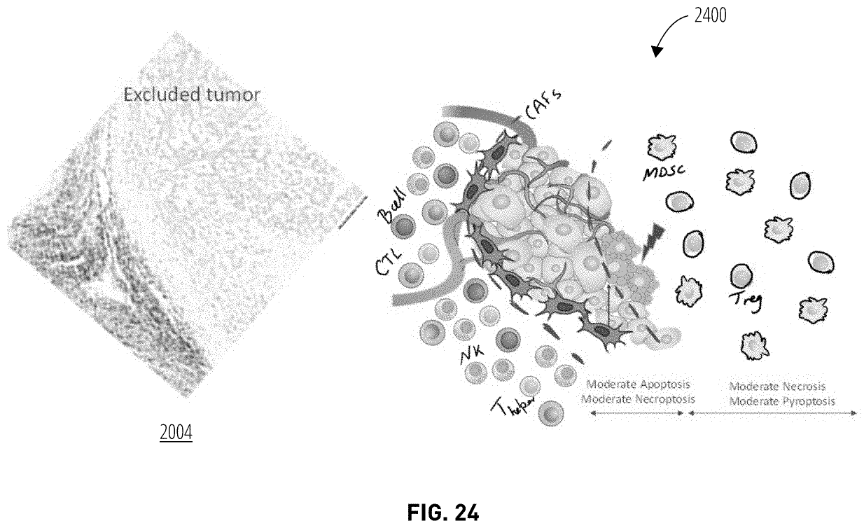

FIG. 24 illustrates a diagram 2400 depicting an excluded tumor 2004. The systems and method of the present disclosure provide that treatment plan 730 for such an excluded tumor 2004 can be generated where the treatment plan 730 includes protocol parameters 724 configured to achieve a zone two 1116 IRE and/or H-FIRE therapy. It is to be appreciated, that zone two 1116 therapies have a mild effect from other ablation modalities. In this case, the angiogenesis has stopped with good control of the vasculogenesis. Therefore, the abnormal perfusion rate is decreased and since there are less competing cancerous cells the immune system can function better. This scenario would represent a combination of anti-inflammatory and proinflammatory cell death mechanisms where close to the electrodes and high intensity electrical fields, the other ablation modalities dependent damage will contribute to necrosis and pyroptosis, however as you go further from the electrodes, the apoptotic and necroptotic cell death mechanisms become more dominant. The whole tumor microenvironment is going through nonhomogeneous cell death mechanisms from center to periphery. The regions of zone 2 that are undergoing apoptosis and necroptosis probably contain TAAs and therefore are easier to be recognized by immature DCs whereas the rest of zone two 1116 is undergoing severe damage from other ablation modalities which may stop angiogenesis and hence provide more oxygen for the coming infiltrated T cells. Since the extent of IRE on the boundary of the tumor will remodel the CAFs, the chance of T cell infiltration in the excluded tumors 2004 is increased.

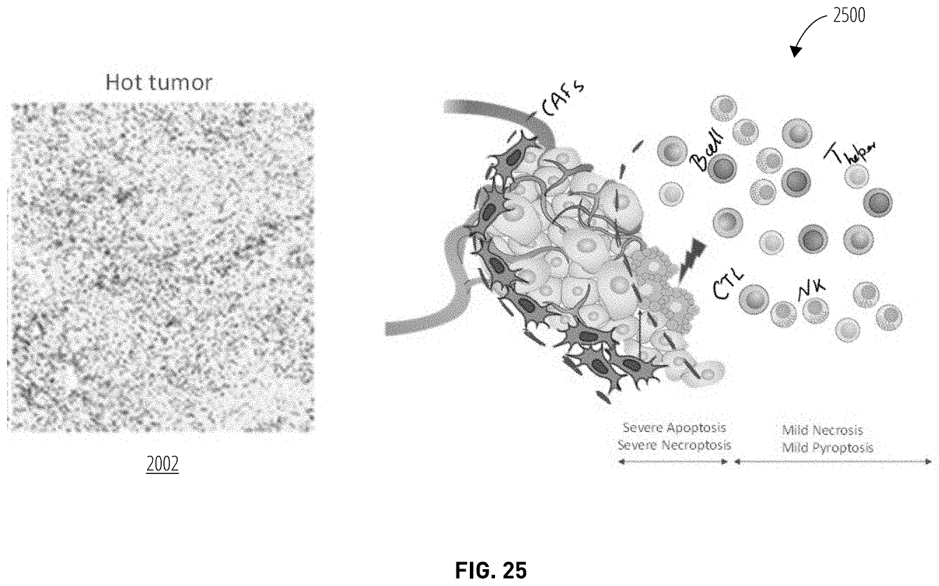

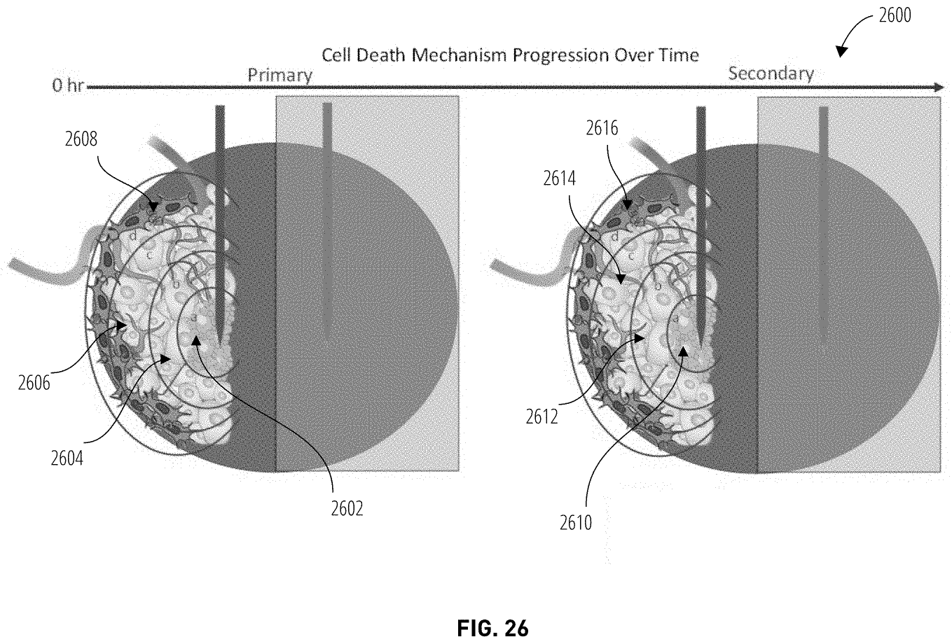

FIG. 25 illustrates a diagram 2500 depicting a hot tumor 2002. The systems and methods of the present disclosure provide that a treatment plan 730 for such a hot tumor 2002 can be generated where the treatment plan 730 includes protocol parameters 724 configured to achieve a zone one 1114 IRE and/or H-FIRE therapy. For example, in a zone one 1114 IRE treatment with no temperature affect, the vasculature structure is preserved such that the blood supply is good and so is oxygenation. The angiogenesis and vasculogenesis decreased due to the relative remodeling in vascular structure. The extent of apoptotic response would induce anti-inflammatory cell death in cancerous cells while preserving the extracellular membrane structure intact. The lack of competing cancerous cells would provide enough oxygen for infiltrated coming T cells. The cancer cells are mostly killed, the TAAs are preserved, and the immature DCs (besides recognizing the TAAs) need oxygen to get to the mature population and initiate the systemic response. If CD4 and CD8 T cells can be infiltrated without presence of MDSC or T regs, the oxygen level would be enough to support the already activated adaptive response. An IRE treatment plan 730 for zone one 1114 applied to hot tumor 2002 may provoke an innate immune response such that the cancer cells and enough T cells are infiltrated into the tumor microenvironment. By usage of zone one 1114 IRE, the effect of primary adjuvant therapies in addition to positive changes to CAFs to support anti-tumor response may be intensified.

As discussed herein, apoptosis has primary and secondary cell death mechanisms. The secondary mechanism, apoptotic necrosis, is mild proinflammatory. Apoptosis is programmed and immunosuppressive so the Treg cells can be produced by conversion of the CD4 cells and change the tumor microenvironment to immunosuppressive microenvironment. FIG. 26 illustrates a diagram 2600 depicting progression of cell death over time from a primary cell death mechanism towards secondary cell death mechanisms as time since application of an IRE and/or H-FIRE procedure is applied. As depicted, necroptosis is programmed and happens quickly because it is not caspase dependent and is proinflammatory, so it disrupts the membrane and the content of the cancer cells is released so the antigens are present for the APCs for a proinflammatory reaction. As such, severe necrosis 2602 transitions to mild necrosis 2610 as time passes. As time passes, the severity of necrosis is lowered because effect the source of the trigger (IRE and/or H-FIRE) is weakened. The pyroptosis mechanism is programmed fever trigged by release of IL-1 leukocyte, proinflammatory. The region that was mostly covered by mild pyroptosis 2604 cell death is going to transform into severe pyroptosis 2612 which is the closest cell death mechanism to necrosis. Additionally, severe necroptosis 2606 transitions to mild necroptosis 2614 and mild apoptosis 2608 transitions to severe apoptosis 2616.

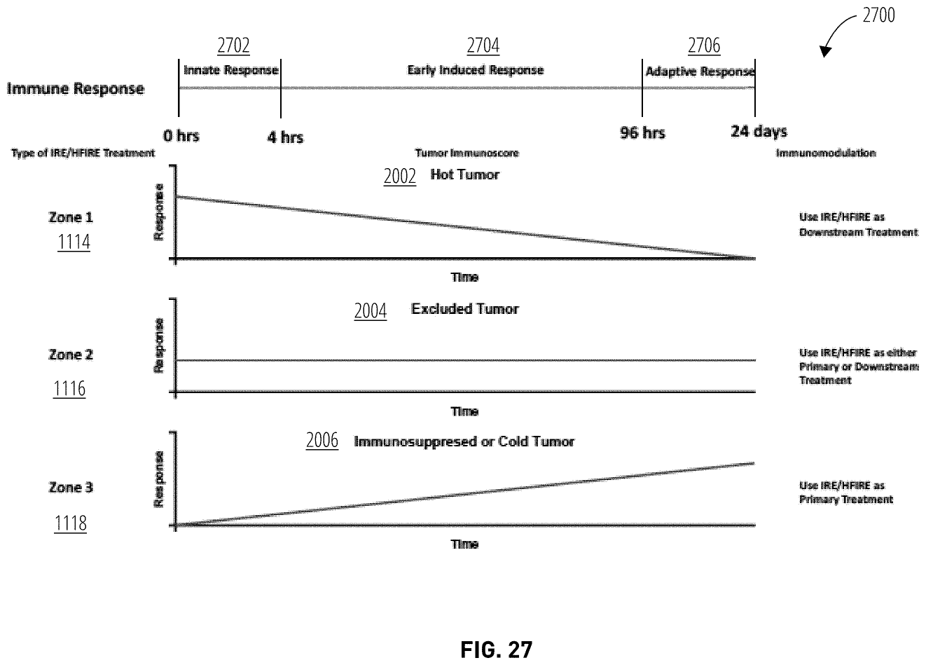

FIG. 27 illustrates a diagram 2700 showing an immune response of various tumor immunity types over time after application (or after initiation) of an IRE and/or H-FIRE therapy. For example, the 2700 depicts an innate response 2702, an early induced response 2704, and an adaptive response 2706. In general, the innate response 2702 may occur within 0 to 4 hours after initiation of an IRE and/or H-FIRE treatment whereas the adaptive response 2706 may occur 96 hours to 24 days post IRE and/or H-FIRE treatment. As can be seen, the immune response of a hot tumors 2002 decreases over time when the tumor is treated with a zone one 1114 treatment, indicating that IRE and/or H-FIRE could be used as a downstream treatment, or rather after application of other adjunctive treatments. The immune response of excluded tumors 2004 is steady over time when the tumor is treated with a zone two 1116 treatment, indicating that IRE and/or H-FIRE treatments can be used either as primary or downstream treatments. Additionally, the immune response of cold tumors 2006 increases over time when the tumor is treated with a zone three 1118 treatment, indicating that IRE and/or H-FIRE treatments can be used as a primary treatment.

FIG. 28 to FIG. 31 depict example treatment plans 730 that can be generated in accordance with the present disclosure. Ablation therapy system 100 and/or ablation therapy device 700 can be arranged to generate these treatment plans, for example, based on implementing routine 2100. It is noted that these examples are provided to illuminate the number of protocol parameters 724 including IRE and/or H-FIRE protocol parameters 724 as well as adjunctive therapy parameters that can be generated for 730 given a tumor immunity type and/or immunoscore as contemplated herein. These examples, however, are not intended to be limiting.

FIG. 28 depicts a diagram 2800 that contemplates potential side effects and best dose of multiantigen deoxyribonucleic acid (DNA) plasmid-based vaccine in treating patients with human epidermal growth factor receptor 2 (HER2)-negative stage III-IV breast cancer. Multiantigen DNA plasmid-based vaccine may target immunogenic proteins expressed in breast cancer stem cells which are the component of breast cancer that is resistant to chemotherapy and has the ability to spread. Vaccines made from DNA may help the body build an effective immune response to kill tumor cells. As can be seen from this diagram 2800, clinician input 728 is received at a computing device (e.g., ablation therapy device 700, or the like). In some examples, clinician input 728 can include indications that the tissue to be treated is immunosuppressed HER2-negative stage III/IV breast cancer model lacks in TAAs & TSAs priming, infiltration of Tcells into the TME, and the check point inhibitory factors necessary to make the treatment effective.

From the clinician input 728, ablation therapy device 700 can determine treatment plan 730 (e.g., using machine learning, or the like, which is outlined in greater detail below). Treatment plan 730 can include a recommendation to use IRE and/or H-FIRE technology at zone two 1116 or zone three 1118 with the intradermal administration of STEMVAC (CD105/Yb-1/SOX2 CDH3/MDM2-polyepitope plasmid DNA vaccine) to prime the TAAs and TSAs.

The treatment plan 730 can further include a prescription to determine whether a STEMVAC T helper 1 cells (Th1) polyepitope plasmid based vaccine elicits a persistent memory T cell response and whether immunity can be further enhanced/maintained by two additional STEMVAC vaccines (boosters) given 3 and 9 months after the priming regimen in the absence of disease progression or unacceptable toxicity. In some embodiments, post IRE and/or H-FIRE evaluation can include evaluating whether STEMVAC vaccination modulates T regulatory cells and myeloid-derived suppressor cells (MDSC).

The treatment plan 730 can further include a post treatment case plan, including for example, a prescription to follow up twice yearly for 5 years. The primary outcome measures are immunologic efficacy defined as achievement of a statistically significant increase in Th1 cell immunity for at least 50% of the immunizing antigens as compared to baseline. The study is designed to record the incidence of toxicity per Cancer Therapy Evaluation Program Common Terminology Criteria for Adverse Events version 4.0. The type and grade of toxicities noted during the immunization regimen will be summarized. Adverse events noted by the investigator/designated clinical research staff will be tabulated according to the affected body system. Descriptive statistics will be used to summarize changes from baseline in clinical and/or laboratory parameters

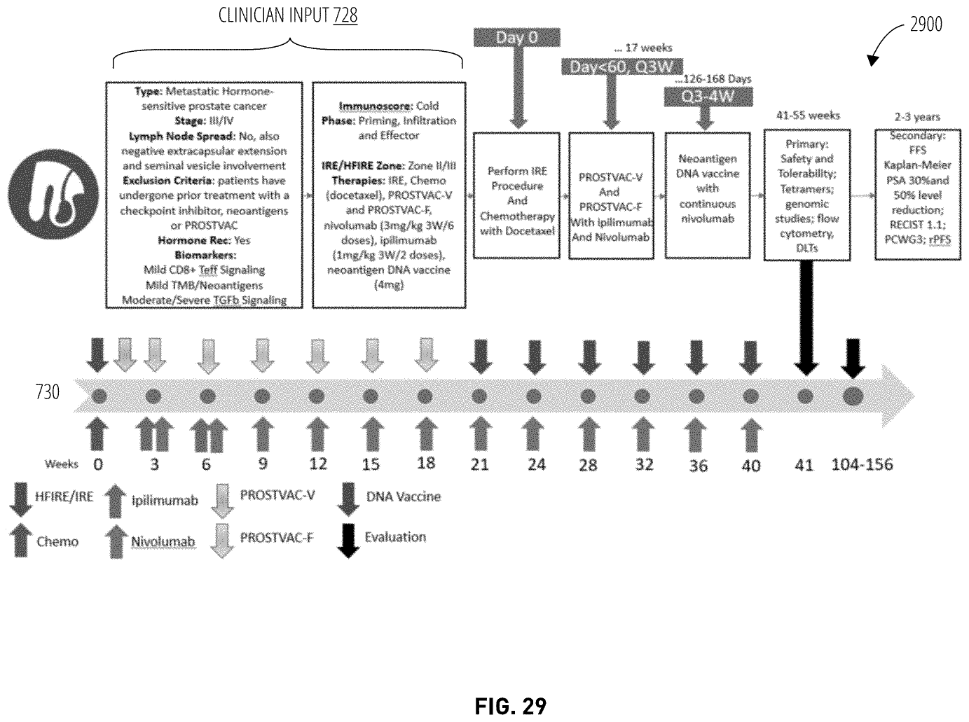

FIG. 29 depicts a diagram 2900 showing an example treatment plan 730 that can be generated based on clinician input 728. In particular, diagram 2900 depicts treatment plan 730 generated to provoke an immune response to IRE and/or H-FIRE at zone two 1116 or zone three 1118 with a shared antigen vaccine (PROSTVAC) and tumor specific antigens generated DNA vaccine in combination with checkpoint blockade using nivolumab (anti-PD-1), and ipilimumab (anti-CTLA-4). Treatment plan 730 can further include recommendations to determine the impact of the combination immunotherapy on peripheral T cell activation, as well as immune response in the tumor microenvironment. Further, treatment plan 730 can include a recommendation to evaluate the safety and tolerability to this novel personalized immunotherapy in combination with checkpoint blockade.

As depicted, diagram 2900 shows an example for cold tumor using a metastatic hormone sensitive prostate cancer model. The IRE and/or H-FIRE with primarily necrotic and pyroptotic cell death mechanisms will be zapping the tumor locally to prime the TAAs with the hope of priming neoantigens too. To support the local IRE and/or H-FIRE priming with a systemic response, treatment plan 730 can incorporate chemo at low dosage. Within 60 days after the last dose of docetaxel, patients will start a priming dose of PROSTVAC-V, and subsequent doses of PROSTVAC-F in combination with ipilimumab (1 mg/kg every 3 weeks for 2 doses), and nivolumab (3 mg/kg every 3 weeks for 6 doses), taking a total of approximately 17 weeks to complete. The ipilimumab function is to inhibit the CTL4, a protein receptor that downregulates the immune system and nivolumab to block the PDL-1. The PROSTVAC-V-Replication-competent vaccinia virus which has been engineered to encode the sequences for a modified human prostate-specific antigen (PSA) and a triad of co-stimulatory molecules (TRICOM). The PROSTVAC-F-Fowlpox virus which does not replicate in human cells and has been engineered to encode the same sequences present in PROSTVAC-V.

Treatment plan 730 further prescribes that the patients will receive a neoantigen DNA vaccine with continuous nivolumab treatment. The vaccine will be administered by intramuscular injection for a total of 6 treatments every 28 days +/−7 days with at least 21 days between injection days. Each DNA vaccination will be 4 mg vaccine administered intramuscularly using a TriGrid electroporation device. Patients will receive nivolumab at 480 mg every 28 days concurrently with neoantigen DNA vaccine. This is Treatment B. In the event the DNA vaccine production is delayed, patients will receive single agent nivolumab at 3 mg/kg every 4 weeks beginning week 21 for up to two interim doses of nivolumab.

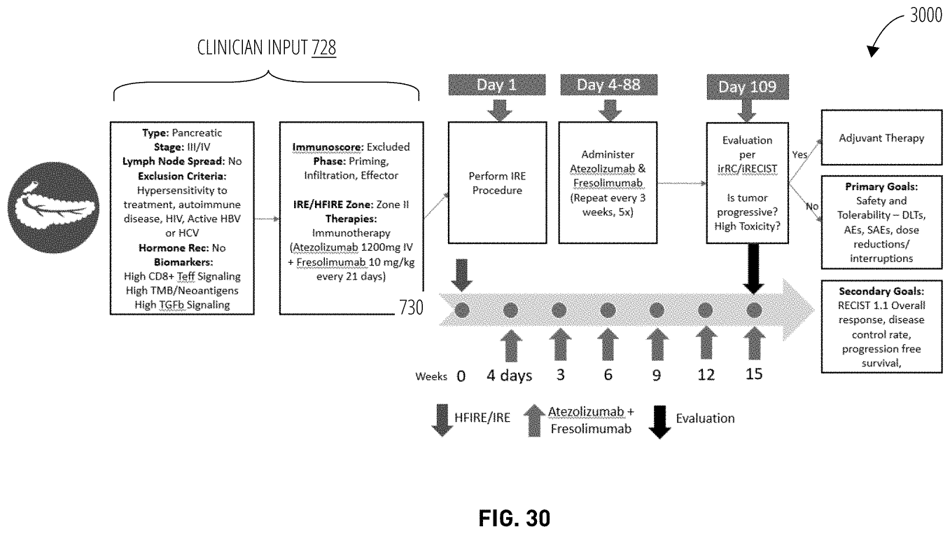

FIG. 30 depicts another treatment plan example in diagram 3000. Diagram 3000 shows stage III or IV pancreatic cancer with no spread to the lymph nodes, high levels of CD8+ signaling and TMB, and high levels of TBFβ signaling are all indicative of a tumor with an excluded immunoscore. Therefore, treatments should be focused on priming, infiltration, and effector phases for the best outcome. IRE/HFIRE at Zone II and a combination of immunotherapies Fresolimumab (anti-TGFβ) and Atezolizumab (anti-PD-1) are selected. The IRE and/or H-FIRE procedure is completed on day 1 and the first dose of the immunotherapies is administered on day 4. Five additional doses of the immunotherapies are administered every 3 weeks until week 15 when the tumor is evaluated following Response Evaluation Criteria in Solid Tumors (RECIST 1.1) or the immune related response criteria (irRC).

Treatment plan 730 prescribes zone two 1116 IRE and/or H-FIRE to promote antigen priming and remodel the CAFS in the tumor stroma while Atezolizumab and Fresolimuab used in combination can reduce TGF-β signaling in stromal cells, facilitate T cell penetration into the center of the tumor, and provoke vigorous anti-tumor immunity and tumor regression. Atezolizumab binds to PD-L1, blocking its binding to and activation of its receptor programmed death 1 (PD-1) expressed on activated T-cells, which may enhance the T-cell-mediated immune response to neoplasms and reverse T-cell inactivation. In addition, by binding to PD-L1, atezolizumab also prevents binding of this ligand to B7.1 expressed on activated T cells, which further enhances the T-cell-mediated immune response. Fresolimumab binds to and inhibits the activity of all isoforms of TGF-beta, which may result in the inhibition of tumor cell growth, angiogenesis, and migration.

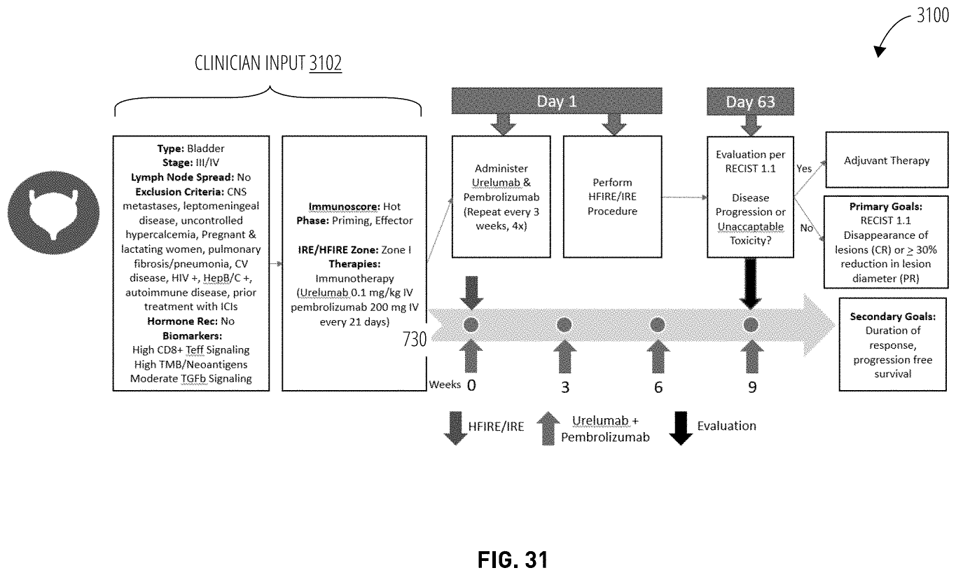

FIG. 31 depicts yet another example treatment plan 730 in diagram 3100. Diagram 3100 shows an example of a treatment plan for a hot tumor. Stage III or IV bladder cancer with no spread to the lymph nodes, high levels of CD8+ signaling and TMB, and moderate levels of TGFβ signaling are all indicative of a tumor with a hot immunoscore. Treatment plan 730 can prescribe IRE and/or H-FIRE at zone one 1114 to focused on the priming and effector phases for best outcome. With some examples, treatment plan 730 can include a recommendation for IRE and/or H-FIRE at zone one 1114 in combination with immunotherapies Urelumab (anti-CD137) and Pembrolizumab (anti-PD-L1). The immunotherapies are first administered the same day as, but prior to, the IRE and/or H-FIRE procedure. Three additional doses of the immunotherapies are administered every 3 weeks until week 9 when the tumor is evaluated following Response Evaluation Criteria in Solid Tumors (RECIST 1.1).

Zone one 1114 IRE and/or H-FIRE kills tumor cells while preserving TAAs and potentially can induce Tumor Specific Antigens (TSA) which might reformulate the vascular structure at stroma with the emphasis on antigen priming and activation of T cells. Pembrolizumab binds to PD-L1, an inhibitory signaling receptor expressed on the surface of activated T cells, and blocks the binding to and activation of PD-L1 by its ligands, which results in the activation of T-cell-mediated immune responses against tumor cells. Urelumab specifically binds to and activates CD137-expressing immune cells, stimulating an immune response, in particular a cytotoxic T cell response, against tumor cells.

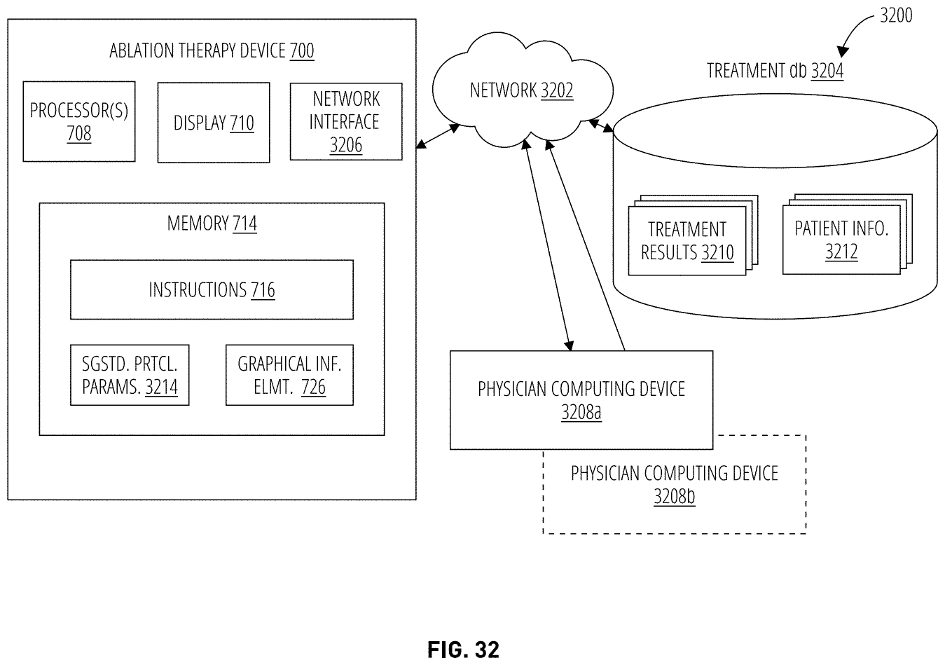

FIG. 32 illustrates an ablation therapy system 3200. In some examples, ablation therapy system 3200 can be implemented to include ablation therapy device 700 of FIG. 7. Although ablation therapy system 3200 is described with respect to ablation therapy device 700, other ablation therapy devices could be implemented in ablation therapy system 3200. Further, it is noted that only a part of ablation therapy device 700 is depicted for brevity.

Ablation therapy system 3200 includes ablation therapy device 700 communicatively coupled to treatment database 3204 and one or more physician computing devices (e.g., physician computing device 3208a, 3208b, etc.) via network 3202. In some examples, network 3202 can include the Internet, a local area network, or a wide area network. In some examples, network 3202 can be a private network, such as, for example accessible via virtual private networking (VPN) and/or otherwise credentialed access to protect information exchanged via network 3202. In some examples, network 3202 can be provided by a clinic, a hospital, a research facility, a university, or the like. Access to the network 3202 can be facilitated by a number of computing communication technologies and can include wired (e.g., Ethernet, or the like) or wireless (e.g., WiFi, 4G, 5G, or the like) communication protocols.

The physician computing devices 3208a and/or 3208 can be any of a variety of computing devices, such as, a laptop, a desktop, a tablet computer, a mobile phone, or the like. Although not shown, physician computing devices 3208a and/or 3208b can include a processing unit, memory comprising instructions executable by the processing unit, a display, and I/O devices. For example, the display can be a touch screen display capable of both displaying graphical information elements and receiving input. It is noted that system 3100 can include just a single physician computing device (e.g., physician computing device 3208a, or the like), for example, where a single physician performs both immunoanalysis, ablation therapy, and adjunctive therapies. In other embodiments, system 3100 can include multiple physician computing devices (e.g., physician computing devices 3208a and/or 3208b).

Treatment database 3204 can be any of a variety of database structures. In some examples, treatment database 3204 can be provided by a cloud computing environment, such as, a cloud data storage provided. With other examples, treatment database 3204 can be provided by a server, a workstation, a cloud computing service, a virtually hosted computing device, a container computing device, or the like. Treatment database 3204 can store indications of treatment results 3208 and/or patient information 3212. In particular, treatment database 3204 can store indications of prior ablation therapy treatments such as, protocol parameters associated with the treatment (e.g., voltage, probe pairs, probe pair placement, voltage pulse details, rounds, etc.), measured current, normalized current, tissue conductivity, normalized tissue conductivity, survivability data (e.g., 1 year survivability statistics, 5 year survivability statistics, etc.), pre and post therapy imaging of the target tissue, or other information related to ablation therapy treatments. Treatment database 3204 can also store indication of patient information as outlined herein.

During operation, ablation therapy device 700 can operate to access treatment database 3204 to receive treatment results 3208 or to add to treatment results 3208. This is described in greater detail below. However, it is noted that the present disclosure provides reasons for such a database. More specifically, as noted conventionally, data related to one ablation therapy treatment cannot easily be compared to data from another ablation therapy treatment. Said differently, current measured during one ablation therapy treatment cannot easily be compared to current measured during another ablation therapy treatment. However, the present disclosure provides to normalize current to a common reference point such that current from one ablation therapy treatment can more easily be compared to current from another ablation therapy treatment. Thus, clinics and clinicians can contribute to treatment database 3204 to build a bank of treatments with which protocol parameters for future treatments may be based.

Furthermore, treatment plans 730 comprising both an ablation modality and adjunctive treatments can be generated from immunoscores and/immunity type and/or other information relevant patient information 3112, which can be stored in treatment database 3204. It is to be appreciated that treatment database 3204 can be multiple databases, perhaps maintained by multiple physicians, clinics, groups, universities, or the like. Further, patient information 3112 can be gathered from multiple sources (e.g., physicians, clinics, groups, universities, etc.). With some examples, the patient information 3112 may be formatted to conform with and/or of satisfy various rules and regulations regarding patient medical information.

In some embodiments, ablation therapy device 700 can receive approval for the treatment plan 730 (or part of the treatment plan 730) from the multiple physician computing devices. As a specific example, ablation therapy device 700 can receive approval for the ablation therapy portion of the treatment plan 730 from the physician computing device associated with the physician responsible for the ablation therapy (e.g., physician computing device 3208a) and can receive approval for the adjunctive therapy portion of the treatment plan 730 from the physician computing device associated with the physician responsible for the adjunctive therapy (e.g., physician computing device 3208b). Further, ablation therapy device 700 can receive approval for the overall treatment plan 730 from the physician computing device associated with the physician responsible for the overall care or treatment of the ablation therapy (e.g., another physician computing device, or the like).

In addition to the components detailed elsewhere herein, ablation therapy device 700 can include a network interface 3206. Ablation therapy device 700 can send and receive data (e.g., information elements, data structures, or the like) to/from treatment database 3204 via network 3202 with network interface 3206. For example, network interface 3206 can format data for transmission over network 3202 via a communication protocol or can decode data transmitted over network 3202 via the communication protocol.

As described in greater details above, ablation therapy device 700 can determine suggested protocol parameters 3210 (e.g., treatment plan 730, protocol parameters 724, etc.) from information stored in treatment database (e.g., patient information 3212, treatment results, 3210, etc.) and can generate graphical information element 726 based on the generated plan and/or parameters. Further, the graphical information element 726 can be communicated to physician computing devices 3208a and/or 3208b and input (e.g., acceptance of, or the like) the generated plan received from the physician computing device 3208a and/or 3208b. This and other examples of the disclosure are described in greater detail below.

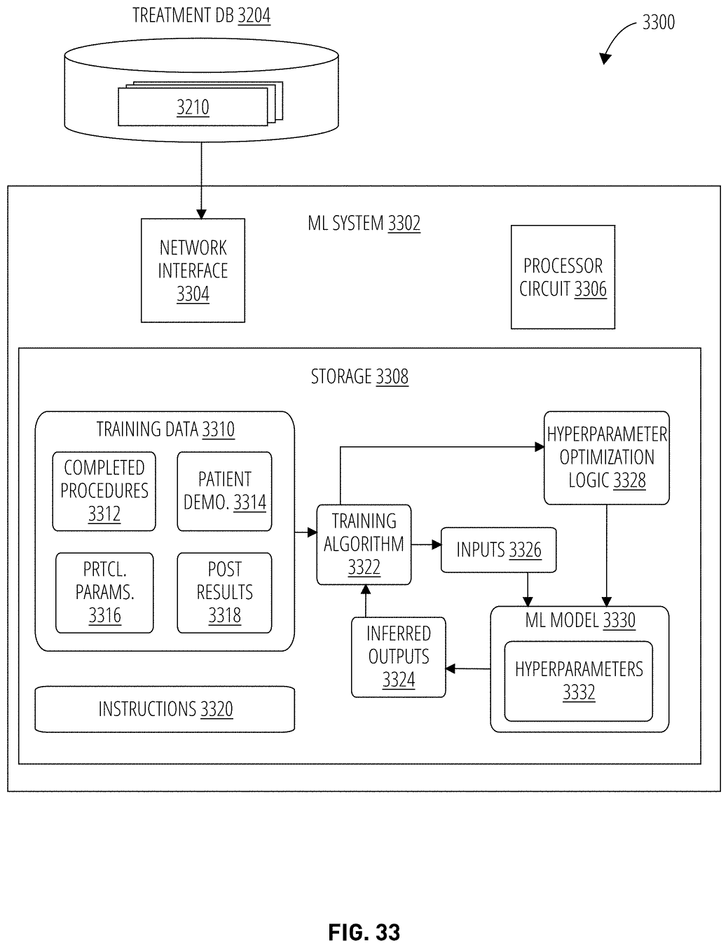

FIG. 33 illustrates a machine learning (ML) environment 3300, in accordance with non-limiting example(s) of the present disclosure. In general, ML environment 3300 can be implemented to apply ML to learn relationships between parameters of an IRE and/or H-FIRE treatment and results of the treatment to predict, or infer, parameters of the IRE and/or H-FIRE procedure. Additionally, ML environment 3300 can be implemented to learn relationships between intensity of IRE and/or H-FIRE treatments and tumor immunity types and/or immunoscores. As a specific example, ML environment 3300 can be implemented to train an ML model to infer current levels, conductivity levels, protocol parameters, or other parameters of an IRE and/or H-FIRE procedure based on a number of inputs such as, voltage pulses applied during the IRE and/or H-FIRE procedure, extrinsic indications about the IRE and/or H-FIRE procedure, or the like. In another specific example, ML environment 3300 can be implemented to train an ML model to infer a treatment plan from immunoscore and/or immunity types of a tumor. In particular, the treatment plan can include protocol parameters to deliver a recommended intensity of IRE and/or H-FIRE to the tumor.

A number of examples of training and using an ML model with an ablation therapy device (e.g., ablation therapy device 700, or the like) are provided herein while describing ML environment 3300. However, prior to providing details of ML environment 3300, it is noted that ML models are generally used in conjunction with an ablation therapy device to generate additional data points for a user (e.g., physician, technician, nurse, or the like) to use in managing a current or active ablation therapy procedure. IRE and/or H-FIRE ablation therapies are typically regarded as more complex than other treatment modalities (e.g., cryogenic therapies, thermal ablation therapies, radio frequency ablation therapies, etc.) by practicing physicians. For example, as described above, using conventional techniques it is difficult to accurately determine progress of treatments in real time, and more specifically when the application of additional electrical pulses causes the mechanism of cell death to change. Said differently, using conventional ablation therapy tools and data available via such tools, it is difficult for a physician to accurately determine when an ablation therapy transitions between therapy zones. Furthermore, it is not currently possible to compare different ablation therapies. That is, for two ablation therapies where the rounds of pulses were applied at different voltage amplitudes, comparing the transition between therapy zones of each individual therapy is not possible. These difficulties in both comparing ablation therapies and determining transitions between therapy zones lead to uncertainty. For example, these difficulties translate to difficulties for the physician to determine whether and when to adjust pulse parameters, whether to continue application of therapeutic pulses or whether to terminate pulse delivery.

The present disclosure provides to train and deploy ML models to generate an inference about an ablation therapy, which can aid a user (e.g., physician, technician, nurse, or the like) in pre-treatment planning, intra-treatment adjustment, and making determination of whether to continue and/or conclude delivery of therapeutic pulses during an ablation therapy. In particular, the present disclosure provides to use ML models combined with the normalized current techniques described above, which is described in greater detail herein. Additionally, the present disclosure provides to train and deploy ML model to generate a treatment plan including an plan an intensity of IRE and/or H-FIRE and optionally other adjunctive therapies.

The ML environment 3300 may include ML system 3302, such as a computing device that applies an ML algorithm to learn relationships between the above-noted items. The ML system 3302 may make use of treatment database 3204, which can be populated as described herein. With some examples, ML environment 3300 can be implemented as part of, or in conjunction with, ablation therapy system 3200. As a specific example, ML system 3302 could be implemented as part of ablation therapy device 700. However, for clarity, ML system 3302 is depicted and described as a separate device from ablation therapy device 700.

As described above, the treatment database 3204 may include information (e.g., patient data, pre-treatment data, treatment parameters, post-treatment data, etc.) collected during actual treatments, and from publicly available data, such as, from studies, registries done to support regulatory approvals, publications, electronic medical records, data repositories of individual medical treatment facilities, regional and national health centers, or the like. The treatment database 3204 may be remote from the ML system 3302 and accessed via a network interface 3304 (e.g., as depicted) or may be stored in a combination of local and remote data storage devices. For example, ML system 3302 may include a storage 3308, which may include a hard drive, solid state storage, and/or random access memory, which can store data associated with treatment database 3204 and treatment results 3208.

Storage 3308 stores training data 3310, which may comprise indications of IRE and/or H-FIRE completed procedures 3312 and patient demographic data 3314 for the patient's undergoing the completed procedures 3312. Training data 3310 can also include indications of protocol parameters 3316 and post procedure results 3318 for the completed procedures 3312. As described in greater detail below, training data 3310 can be generated from data represented in treatment database 3204.

In general, protocol parameters 3316 can be representative of parameters related to planning the IRE and/or H-FIRE treatment. For example, protocol parameters 3316 can include indications of voltage amplitude, total number of voltage pulses, length of planned voltage pulses, information describing a train of voltage pulses, total on time, information describing a burst or bursts of voltage pulses, information describing cycles of voltage pulses, delay between voltage pulses, number of probes, probe type, spacing between probes, information describing a pattern or patterns of probe placement, probe polarity, relative to target (bracket vs. center), exposed length of the electrode(s), dimensions of the targeted ablation area, voltage/cm setting(s), model number of the voltage generator and/or ablation therapy device, software version of the voltage generator and/or ablation therapy device. As another example, protocol parameters 3316 can include indications about the IRE and/or H-FIRE procedure itself, such as, for example, cardiac sync, whether the procedure is open or closed, whether a paralytic is being used, indications of the initial conductivity of tissue (e.g., based on pre-treatment tests, or the like). With still other examples, protocol parameters 3316 could include indications of any number of the electric pulse parameters discussed above with respect to Table 2.

Patient demographic data 3314 can include indications of the demographics for the patient undergoing the completed procedures 3312. For example, patient demographic data 3314 can include indications of age, gender, race, insurance information, diagnosis, organ, cancer type, cancer stage, previous treatments, ongoing treatments (e.g., chemotherapies, focal therapies, or the like), co-morbidity scores, patient vitals (e.g., blood pressure, heart-rate, weight, height, or the like), location of cancer within organ, number of lesions, immune scores, tissue immunity type, imaging studies, implants, tumor location including non-tumor anatomical structures or proximate or in treatment zone (e.g., vessels, organs, bones, or the like), target tissue abnormalities (e.g., cysts, calcification, scar tissue, or the like).

Completed procedures 3312 can include data related to the actual procedure performed, such as, inter-procedure data and end of procedure data. For example, completed procedures 3312 can include indications of start and/or stop time of the procedure, the overall length of time of the procedure, data related to overcurrent conditions during the procedure (e.g., number of overcurrent conditions, amplitude of overcurrent conditions, or the like), adjustments made to any of the protocol parameters during the IRE and/or H-FIRE procedure, actual ones of the protocol parameters 3316 delivered during the IRE and/or H-FIRE procedure (e.g., total number of pulses delivered, number of pulses per probe pair, probe pair pulsing sequence, total pulse on time, current and voltage readings for each pulse delivered, or the like), probe repositioning info, intra or post procedure tissue information (e.g., resistance, conductivity readings, or the like), patient vitals during procedure, cardiac readings, procedural complications including mechanical damage due to probe insertion and/or adjustment, thermal heating, final ablation volumes and/or sizes, tissue conductivity changes not due to electrical pulses (e.g., saline flush, amount of intracellular fluids in target area, or the like). Furthermore, completed procedures 3312 can include indications of normalized current and/or normalized conductivity derived as outlined herein.

Post procedure results 3318 can include indications of information related to results of the procedure, such as, treatment complications (e.g., short term complications, long term complications, or the like), length of hospital stay, recovery times, survival rate (e.g., short term survival, long term survival, or the like), cancer recurrence, time to recurrence, disease free statistics, metastatic disease, quality of life measures, or the like.

Training data 3310 can be generated by ML system 3302. For example, processor circuit 3306 can execute instructions 3320 to generate training data 3310 from treatment results 3208 stored in treatment database 3204. In general, the training data 3310 may be applied to train ML model 3330. Depending on the particular application, different types of ML models 3330 may be suitable for use. For instance, in the depicted example, an artificial neural network (ANN) may be particularly well-suited to learning associations between completed procedures 3312, patient demographic data 3314, protocol parameters 3316, and post procedure results 3318. Convoluted neural networks (CNNs) and random forest networks may also be well-suited to this particular type of task. However, one of ordinary skill in the art will recognize that different types of ML models 3330 may be used, depending on design goals, the resources available, the size of the dataset of training data 3310, etc.

Any suitable training algorithm 2420 may be used to train the ML model 3330. Nonetheless, the example depicted in FIG. 33 may be particularly well-suited to a supervised training algorithm or reinforcement learning. For a supervised training algorithm, the ML system 3302 may apply inputs 3326 to the ML model 3330 while the model generates an inference, such as, inferred outputs 3324 based on the inputs 3326. In some examples the completed procedures 3312, patient demographic data 3314, and post procedure results 3318 can be applied as inputs 3326 to map these portions of training data to protocol parameters 3316. It is to be appreciated that the aim of “training” the ML model 3330 is for the ML model 3330 to learn associations between the inputs 3326 (e.g., completed procedures 3312, patient demographic data 3314, and post procedure results 3318) and inferred outputs 3324 (e.g., protocol parameters 3316, treatment plan 730, etc.).

ML models (e.g., ML model 3330) have hyperparameters 3332.

Hyperparameters 3332 can include a variety of items related to the ML model 3330, such as, for example, number of nodes, number of layers, number of hidden layers, value of weights connecting each node, the activation function of each node, the learning gradient, etc. In a reinforcement learning scenario, hyperparameters 3332 of the ML model 3330 are adjusted, based on the training algorithm 3322 with the goal being that the inferred outputs 3324 converge upon an acceptable level of accuracy to what the inferred outputs 2428 are expected to be.

The training algorithm 3322 may be applied using processor circuit 3306, which may include suitable hardware processing resources that operate on the logic and structures in the storage 3308. As noted, training algorithm 3322 and/or the development of the trained ML model 3330 is at least partially dependent on model hyperparameters 3332. In exemplary examples, the model hyperparameters 3332 can be automatically selected based on hyperparameter optimization logic 3328, which may include any known hyperparameter optimization techniques as appropriate to the ML model 3330 selected and the training algorithm 3322 to be used.

In some embodiments, some of the training data 3310 may be used to initially train the ML model 3330 while some of the training data 3310 can be reserved and used as a validation subset. The portion of the training data 3310 not including the validation subset may be used to train the ML model 3330 whereas the validation subset may be used to test the trained ML model 3330 and to verify that the ML model 3330 is able to generalize or correctly infer outputs from unseen or new data.

In optional examples, the ML model 3330 may be re-trained over time, for example, to accommodate knowledge about updated, new, recent, or otherwise different procedures and associated protocol parameters not reflected in the training data 3310 with which the ML model 3330 was previously trained on. As a specific example, ML model 3330 can be repeatedly (e.g., on a fixed period, as sufficient new data exists, or the like) trained to account for various updates in the data set (e.g., updates in physician preferences, updates in accepted treatment guidelines, new academic research, new clinical trials or studies, or the like). Along these lines, with many examples, treatment database 3204 can be expanded over time. Furthermore, with some examples, portions of treatment database 3204 (e.g., completed procedures 3312, patient demographic data 3314, protocol parameters 3316, or the like) can be populated around the time of an IRE and/or H-FIRE procedure while other portions (e.g., post procedure results 3318, or the like) of treatment database 3204 can be populated subsequent to the procedure, possibly by a different user (e.g., different physician, different technician, different nurse, or the like). As such, an updated version of training data 2410 can be generated from an expanded treatment database 3204.

Once the ML model 3330 is trained, it may be executed, for example, by processor circuit 3306 (or another processor circuit, such as, processor(s) 708 of ablation therapy device 700) to new input data. As a specific example, ML model 3330 can be executed by processing circuitry of an ablation therapy device to generate an inference about protocol parameters 3316 from inputs related to a current IRE and/or H-FIRE procedure for which the ablation therapy device is to be used. This input to the ML model 3330 may be formatted according to a predefined format, which for example, can mirror the way that the training data 3310 was provided to the ML model 3330. The ML model 3330 may generate inferred outputs 3324 which may be, for example, a prediction of normalized currents, tissue conductivities, protocol parameters, a treatment plan, or the like based on the provided inputs.

The inferred outputs 3324 may be provided to a user of the ablation therapy device (e.g., physician, nurse, technician, or the like) as a recommendation for an overall treatment plan including protocol parameters to select for a current IRE and/or H-FIRE procedure or as another data point to use in adjusting and/or concluding the procedure.

The above description pertains to a particular kind of ML system 3302, which applies supervised learning techniques given available training data with input/output pairings.

However, the present disclosure is not limited to use with a specific ML paradigm, and other types of ML techniques may be used. For example, in some embodiments the ML system 3302 may apply other types of ML techniques, such as evolutionary algorithms, without departing from the scope of the disclosure.

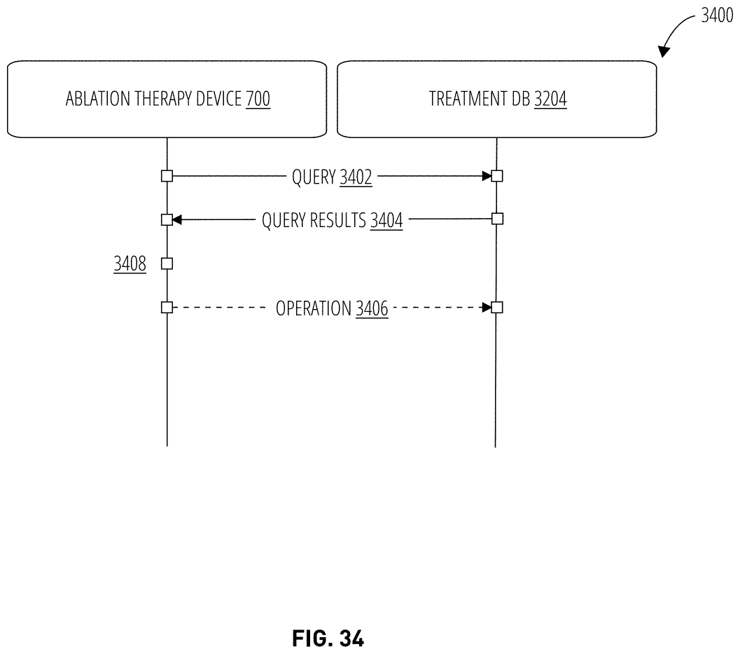

FIG. 34 illustrates technique 3400 detailing operations for ablation therapy device 700 and/or treatment database 3204 according to examples of the present disclosure. In technique 3400, at operation 3402, ablation therapy device 700 can send a query to treatment database 3204. Likewise, at operation 3402 treatment database 3204 can receive a query from ablation therapy device 700. For example, processor(s) 708 in executing instructions 716 can generate a query for treatment database 3204 and can send the query to treatment database 3204 via network interface 3206 and network 3202. As a specific example, the query can include a request to provide treatment results 3208 for a particular target tissue 112 (e.g., pancreas, prostrate, breast, lung, liver, kidney, or the like). With some examples, ablation therapy device 700 can generate query based on input received from a clinician. For example, processor(s) 708 in executing instructions 716 can receive input from a clinician indicating a type of target tissue 112. At operation 3404, treatment database 3204 can send query results to ablation therapy device 700. Likewise, at operation 3404, ablation therapy device 700 can receive query results from treatment database 3204. For example, processor(s) 708 in executing instructions 716 can receive results to a query (e.g., query send at operation 3402, or the like) from treatment database 3204.

At operation 3408, ablation therapy device 700 can generate graphical information element 726 including indications of treatment results 3208 and/or suggested protocol parameters 3210. However, in general, processor(s) 708 in executing instructions 716 can generate a graphical display (e.g., a plot, multiple plots, or the like) including indications of treatment results 3208. As another example, processor(s) 708 in executing instructions 716 can generate suggested protocol parameters 3210 and a graphical display including indications of suggested protocol parameters 3210. With some examples, suggested protocol parameters 3210 can be generated based on treatment results 3208.

At operation 3406, ablation therapy device 700 can send data including indications of an ablation therapy treatment to treatment database 3204. Likewise, at operation 3406 treatment database 3204 can receive a data from ablation therapy device 700 including indication of an ablation therapy treatment. Furthermore, treatment database 3204 can add the received ablation therapy treatment to treatment results 3208. With some examples, information communicated to 1502 at operation 3406 can include normalized current estimated electrical conductivity, pre and post imaging analysis, survivability results, etc.

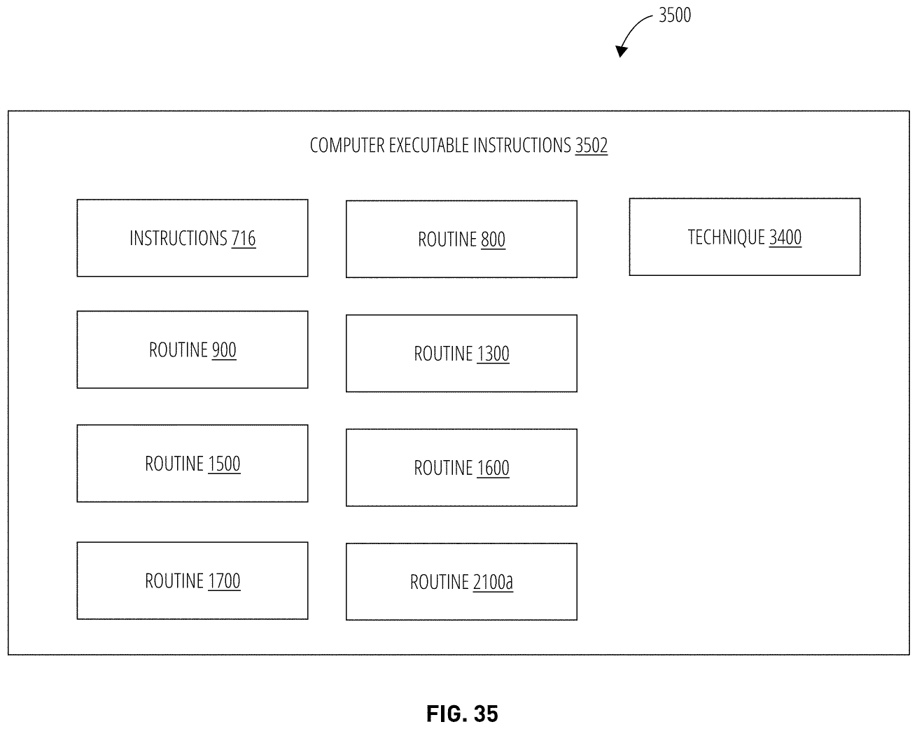

FIG. 35 illustrates computer-readable storage medium 3500. Computer-readable storage medium 3500 may comprise any non-transitory computer-readable storage medium or machine-readable storage medium, such as an optical, magnetic or semiconductor storage medium. In various embodiments, computer-readable storage medium 3500 may comprise an article of manufacture. In some embodiments, 700 may store computer executable instructions 3502 with which circuitry (e.g., processor(s) 708, or the like) can execute. For example, computer executable instructions 3502 can include instructions to implement operations described with respect to instructions 716, routine 800, routine 900, routine 1300, routine 1500, routine 1600, routine 1700, routine 2100, and/or technique 3400. Examples of computer-readable storage medium 3500 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 3502 may include any suitable type of code, such as source code, compiled code, interpreted code, executable code, static code, dynamic code, object-oriented code, visual code, and the like.

FIG. 36A to FIG. 36D illustrates illustrate example graphical user interfaces (or GUIs). For example, these figures depict GUIs 3600a, 3600b, 3600c, and 3600d, respectively, which can be generated by an ablation therapy device and presented on a display for a clinician. For example, ablation therapy device 700 can generate GUIs 3600a, 3600b, 3600c, and/or 3600d and present such GUIs on display 710. As a specific example, processor(s) 708 in executing instructions 716 can generate display data (e.g., display frames, or the like) comprising an indication of treatment results and/or suggested protocol parameters 3210. For example, ablation therapy device 700 can generate tissue selection GUI input elements 3602. For example, input elements may comprise any of the following: tissue type (i.e., pancreas, heart, prostate, breast, lung, liver, kidney); treatment parameters (i.e., RE, IRE or H-FIRE, probe type, number of probes, probe spacing, waveform parameters, number of pulses, electrode exposure length, treatment zone size, margin size); and/or physical change inputs (i.e., probe reposition, electrode exposure length change, or pull back length). Ablation therapy device 700 can receive input from a clinician (e.g., clinician input 728, or the like) regarding type of target tissue 112 via tissue selection GUI input elements 3602. Further, ablation therapy device 700 can generate graphical display elements including one or more plots or suggestions for an ablation therapy treatment for the type of target tissue 112. For example, a plot 3604 comprising indication of normalized current from treatment results 3208 having a same or similar target tissue type can generated and included in GUI 3600a. As another example, a plot 3606 comprising indication of a rate of change of normalized current from treatment results 3208 having a same or similar target tissue type can be generated and included in GUI 3600a. In yet another example, a plot 3608 comprising indication of a normalized tissue conductivity from treatment results 3208 having a same or similar target tissue type can be generated and included in GUI 3600a. With yet another example, a plot 3610 comprising indication of combined treatment data from multiple physicians (e.g., clinicians, or the like) in treatment results 3208 having a same or similar target tissue type can be generated and included in GUI 3600a. As another example, the parameter settings (extrinsic) can be also put in the matrix of information. In general, any parameters that is listed in Equation 1 can be part of this matrix of information. Some text in FIGS. 18(A)-18(D) (Rate of Change of Normalized Conductivity vs. Pulse number, and Normalized Conductivity vs. Voltage Gradient (v/cm)) describe potential future plots or charts that may not be shown, including but not limited to, other extrinsic factors including shape factor, electrode length, distance between electrodes/probes.

In some examples, as depicted, GUI 3600a can include multiple plots (e.g., plot 3604, plot 3606, plot 3608, plot 3610, or the like). In other examples (not shown), GUI 3600a can include a single plot. With still other examples (not shown), GUI 3600a could include actual suggested parameters (e.g., 4 rounds of 60 pulses each at 2100 volts, or the like) for an ablation therapy treatment for the same or similar target tissue type. With some examples (not shown), the suggested protocol parameters 3210 can be generated based on treatment results 3208 having the highest survivability rates. In other examples (not shown), the suggested protocol parameters 3210 can be generated based on treatment results 3208 where the treatment concluded in a desired treatment zone.

FIG. 36B depicts GUI 3600b showing plots 3604, 3606, 3608, and 3610 as well as GUI input elements 3602 in an alternative arrangement to that depicted in FIG. 36A. FIG. 36C and FIG. 36D illustrate GUI 3600c and GUI 3600d, respectively, which can be generated by an ablation therapy device and presented on a display for a clinician. For these examples, ablation therapy device 700 received an input from a clinician (e.g., clinician input 728, or the like) regarding a different type of target tissue 112, as compared to FIGS. FIG. 36A and FIG. 36B, via tissue selection GUI input elements 3602. In this example user selected a target tissue 112 of prostate tissue. The data used to generate plots 3604, 3606, 3608, and 3610 reflects this change in tissue type 112 and can be used to depict the same type of information to user as described above.

FIG. 36B and FIG. 36D illustrate one example of the flow (represented by arrows) of a user selection of GUI input elements 3602. For example, user may first select a tissue type (including but not limited to pancreas; prostate; breast; lung; liver; and/or kidney), then various treatment parameters (including but not limited to IRE or HFIRE; probe type, number of probes, probe spacing, waveform, number of pulses, and/or electrode exposure), and then physical changes that may occur during a typical procedure (including but not limited to probe reposition round; exposure change; and/or pull back round).

In one example, normalized current and/or normalized conductivity plots depicted in FIG. 36A to FIG. 36D may include, but not limited to, providing the user with the following (i) recommended voltage setting for a procedure; (ii) an expected number of pulses before transition between zones one and two and/or between zones two and three; (iii) the onset and/or transition between the difference mechanism(s) of cell death (i.e., transition between irreversible electroporation, thermal ablation, and/or electrolysis); and/or (iv) when to stop treatment based on a desired outcome. Furthermore, plot indicating the rate of change of normalized current can be used to select treatment options/parameters based on a desired intensity level of irreversible electroporation.

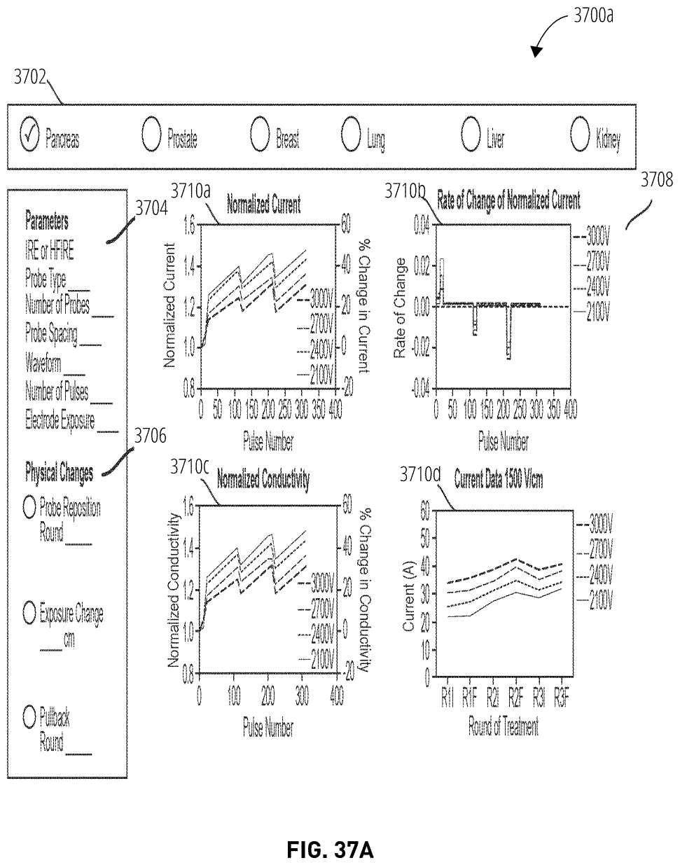

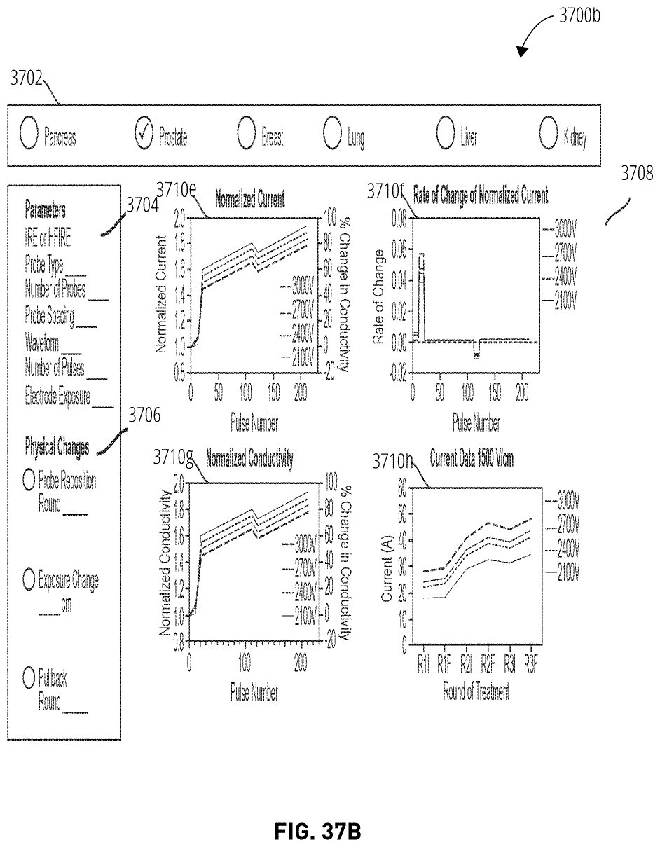

FIG. 37A and FIG. 37B illustrate GUI 3700a and GUI 3700b, respectively, which can be generated by an ablation therapy device (e.g., ablation therapy device 700, or the like) and presented on a display for a clinician to use in pre-planning, intra-treatment adjustment, and conclusion of an IRE and/or H-FIRE procedure. For example, ablation therapy device 700 can generate graphical display GUI 3700a and/or graphical display GUI 3700b and present on display 710. As a specific example, processor(s) 708 in executing instructions 716 can generate display data (e.g., display frames, or the like) comprising the graphical elements represented in these figures.

An ablation therapy device (e.g., ablation therapy device 700, or the like) can generate graphical display GUI 3700a or graphical display GUI 3700b comprising an indication of a tissue type selector 3702, a parameter selector 3704, an adjustment selector 3706, and a treatment characteristic 3708. Furthermore, ablation therapy device 700 can be configured, as described above, to receive an indication of a tissue type (e.g., from a physician, or the like) and generate a graphical display comprising the received tissue type. For example, FIG. 37A depicts displaying an indication of a selected tissue type pancreas in tissue type selector 3702 while FIG. 37B depicts displaying an indication of a selected tissue type prostate in tissue type selector 3702. It is noted that the available tissue types for selection can be depended upon the tissue types represented in treatment database 3204 or the tissue types with which ML model 3330 is trained and that the tissue type examples depicted in these figures is given for example only.

The ablation therapy device 700 can further be configured, as described above, to receive an indication of treatment parameters (e.g., IRE or H-FIRE, probe type, number of probes, probe spacing, waveform parameters, number of pulses, electrode exposure length, treatment zone size, margin size, or the like) from a user (e.g., physician, or the like). Ablation therapy device 700 can generate GUI 3700a and/or GUI 3700b comprising the treatment parameters selected by the user via parameters parameter selector 3704.

Ablation therapy device 700 can generate GUI 3700a and/or GUI 3700b including treatment characteristics 3708. As depicted, treatment characteristics 3708 comprises a number of plots associated with an ablation therapy treatment having the parameters indicated in parameter selector 3704 for the type of tissue reflected in tissue type selector 3702. With some examples, data (e.g., plots 3710a, 3710b, 3710c, and/or 3710d, or the like) depicted in treatment characteristics 3708 can be generated by ML model 3330 based on input from tissue type selector 3702 and parameters parameter selector 3704. As a specific example, as depicted in these figures, plots 3710a, 3710b, 3710c, and 3710d can be generated (e.g., by ML model 3330) comprising an indication of normalized current versus pulse number (e.g., plots 3710a), normalized tissue conductivity versus pulse number (e.g., item 3710b), a rate of change of normalized current versus pulse number (e.g., item 3710c), and a voltage gradient (V/cm) versus a round (number of pulses) of treatment (e.g., item 3710d). It is noted that the depicted plots are given for example only and different plots or more or less plots than the depicted can be generated and displayed in GUI 3700a and/or GUI 3700b.

During an active treatment, ablation therapy device 700 can repeatedly (e.g., on a fixed period, after a number of pulses, after voltage or current level thresholds, or the like) update treatment characteristics 3708. For example, ML model 3330 can be arranged to generate updated plots, such as, updated versions of (e.g., plots 3710a, 3710b, 3710c, and 3710d) based on intra-treatment measurements. For example, ML model 3330 can be configured to generate updated plots based on intra-treatment extrinsic measurements (e.g., current, voltage, or the like). Furthermore, ablation therapy device 700 can further be configured, as described above, to receive an indication of changes to be made intra-treatment from a user (e.g., physician, or the like). As an example, ablation therapy device 700 can be configured to receive, intra-treatment, indications to stop the procedure, stop delivery of therapeutic pulses for one or multiple probe pairs, deactivate one or multiple probe pairs, reactivate one or multiple probe pairs, reactivate delivery of therapeutic pulses, adjust electrocardiogram (ECG) leads, account for repositioned probes, account for changed electrode exposure length, adjust therapeutic pulse parameters (e.g., voltage amplitude, voltage/cm, total number of pulses, pulse width, maximum allowable current and/or conductivity, intrapulse delay, polarity of pulses, delay between sequences or trains of pulses, or the like).

Ablation therapy device 700 can generate GUI 3700a and/or GUI 3700b comprising the intra-treatment changes via adjustment selector 3706. Furthermore, ablation therapy device 700 can update treatment characteristics 3708 based on the changes reflected in adjustment selector 3706. For example, ML model 3330 can be arranged to generate updated plots, such as, updated versions of (e.g., plots 3710a, 3710b, 3710c, and 3710d) based on inputs from tissue type selector 3702, parameters parameter selector 3704, adjustment selector 3706, and/or other extrinsic data related to the active ablation therapy.

As noted above, the complexity of ablation therapies often leads to difficulty in a therapy provider (e.g., clinician, or the like) making a determination of how to adjust a therapy intra-treatment as well as when to conclude a therapy. The present disclosure provides more than merely collecting, analyzing, and displaying information related to an ablation therapy.

Instead, the present disclosure provides a unique system wherein data from different types of IRE and/or H-FIRE treatments having different protocols (intrinsic and extrinsic values) can be compared, such as, via normalized current. Given the details provided herein regarding normalized current, ML model 3330 can be trained on many different ablation therapies (e.g., a reflected in treatment database 3204) even where these ablation therapies used different parameters. Accordingly, information about therapy zones for a current ablation therapy can be generated where such information is not available conventionally. Said differently, the information generated herein and displayed in treatment characteristics 3708 is not information that is conventionally available to collect and analyze. Furthermore, as provided herein ML model 3330 can be trained to generalize normalized currents from any combination of input parameters based on normalized currents for prior therapies with outcomes meeting selected criteria (e.g., as reflected in treatment database 3204). It is emphasized that this is not conventionally possible and is significantly more than merely collecting, analyzing, and displaying information.

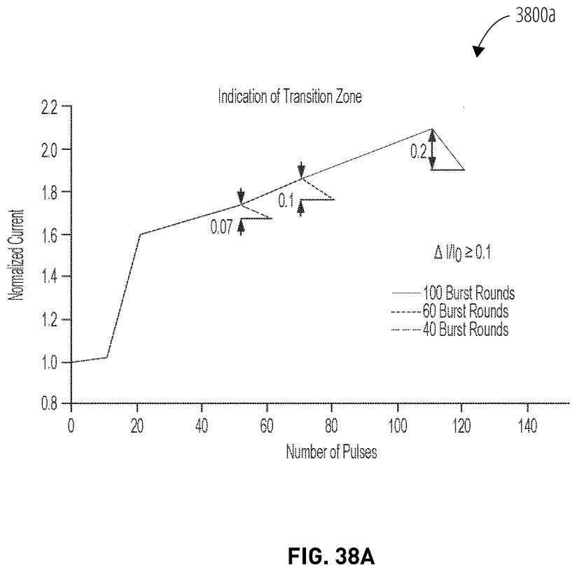

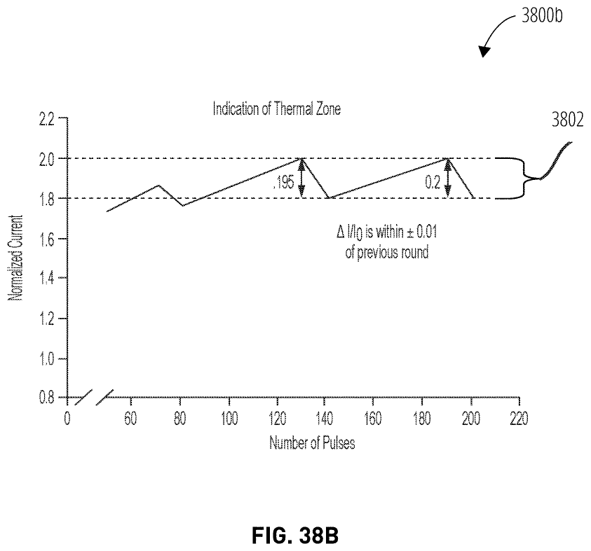

As noted, in some examples, treatment characteristics 3708 can include more or less plots than depicted in GUI 3700a and GUI 3700b of FIG. 37A and FIG. 37B. For example, FIG. 38A and FIG. 38B depict examples of plots that can be generated by ML model 3330 from inputs reflected in tissue type selector 3702, parameters parameter selector 3704, adjustment selector 3706, and/or other extrinsic data. A graphical display (e.g., GUI 3700a and/or GUI 3700b) can be generated to include indications of these plots. Said differently, ML model 3330 can be trained to infer normalized current for an ablation therapy to account for extrinsic and/or intrinsic characteristics of the ablation therapy. Ablation therapy device 700 can generate, based on the inference of normalized current from ML model 3330, plots representing drops in normalized current (e.g., per pulse, per rounds of pulses, or the like) to aid a user in conducting the ablation therapy, such as with pre-treatment planning, intra-treatment adjustments, and treatment conclusion determinations.

FIG. 38A depicts plot 3800a. Plot 3800a is a representation of normalized current for a number of pulses where indications of transition between therapy zones for different number of pulses per round is depicted. Specifically, plot 3800a depicts normalized current and where a transition between therapy zones will occur for 40 burst rounds, 60 burst rounds, and 100 burst rounds. With some examples, ML model 3330 can be configured to generate plot 3800a, as described above, and ablation therapy device 700 can generate treatment characteristics 3708 including an indication of plot 3800a.

As a specific example, ablation therapy device 700 can be arranged to generate (e.g., based on inferences of ML model 3330) a depiction including the plot 3800a. A user (e.g., a physician, or the like) may use the information provided in plot 3800a to determine parameters to use for an ablation therapy with the ablation therapy device 700. In some therapies, transition between therapy zones is indicated by a drop in normalized current of greater than or equal to 0.1. Accordingly, if the user wanted to cause a transition between therapy zones (e.g., between zone one 1114 and zone one 1114, or the like) the user could select parameters with which plot FIG. 38A indicates would cause a drop in normalized current of greater than or equal 0.1. Specifically, a user might select multiple rounds of 40 pulses (e.g., 40 burst rounds) or a single round of 60 pulses or 100 pulses (e.g., a 60 burst round or a 100 burst round) to achieve the desired drop in normalized current of greater than or equal to 0.1. In some examples, a user may elect to apply multiple (e.g., 2, etc.) rounds of 40 pulses to potentially reduce a thermal rise in the tissue due to the delay between rounds. However, another user may elect to apply a single round of 60 or 100 pulses in order to transition between the therapy zones more quickly.

FIG. 38B depicts plot 3800b. Plot 3800b is a representation of normalized current for a number of pulses where, based on the number of pulses, an ablation therapy may exit the IRE therapy zone. Specifically, plot 3800b depicts normalized current for a number of potential pulses and where a transition out of a therapy zone 3802 may be, based on the normalized current. With some examples, ML model 3330 can be configured to generate plot 3800b, as described above, and ablation therapy device 700 can generate treatment characteristics 3708 including an indication of plot 3800b. Said differently, ML model 3330 can be arranged to infer normalized current and ablation therapy device 700 can be arranged to generate a depiction including the plot 3800b from the inferred normalized current. A user (e.g., a physician, or the like) may use the information provided in plot 3800b to determine when to conclude an ablation therapy conducted with the ablation therapy device 700. In some therapies, transition out of a therapy zone is indicated by a drop in normalized current after a round of pulses of less than or equal to 0.01 of the drop in normalized current after the previous round of pulses. For example, transition in therapy zone 3802 shows a drop in normalized current of less than or equal to 0.01 the previous drop in normalized current. Accordingly, if the user wanted to conclude an ablation therapy at the transition, the user could elect to stop the ablation therapy after the number of pulses indicated in plot 3800b corresponding to the transition.

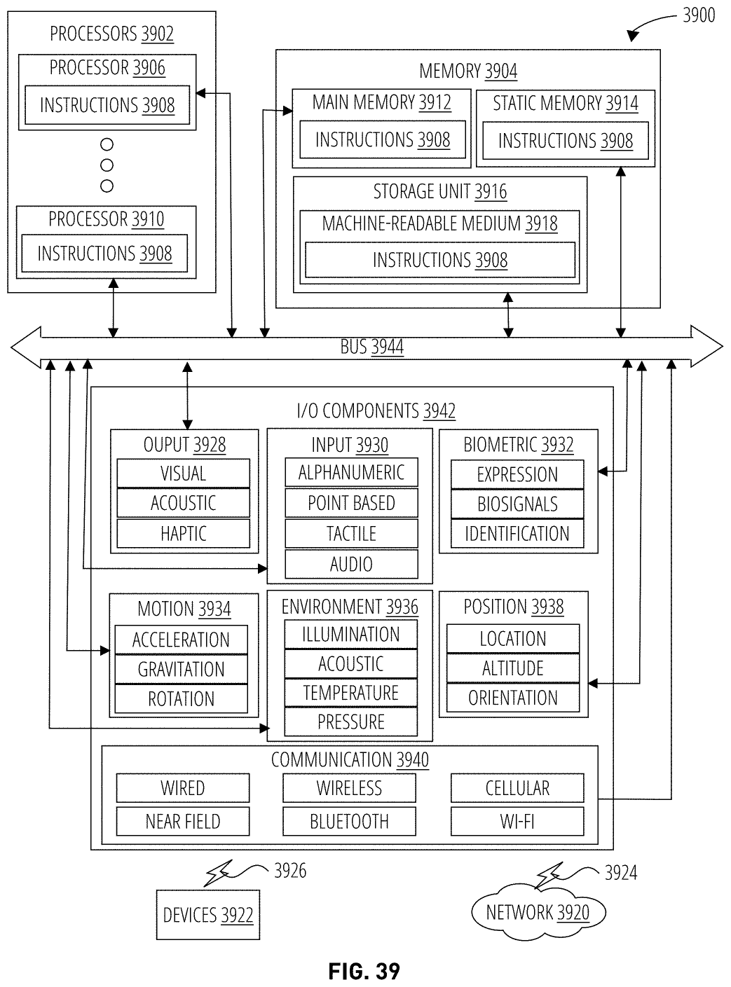

FIG. 39 illustrates a diagrammatic representation of a machine 3900 in the form of a computer system within which a set of instructions may be executed for causing the machine to perform any one or more of the methodologies discussed herein. More specifically, FIG. 39 shows a diagrammatic representation of the machine 3900 in the example form of a computer system, within which instructions 3908 (e.g., software, a program, an application, an applet, an app, or other executable code) for causing the machine 3900 to perform any one or more of the methodologies discussed herein may be executed. For example, the instructions 3908 may cause the machine 3900 to execute instructions 716 of FIG. 7, routine 800 of FIG. 8, routine 900 of FIG. 9, routine 1300 of FIG. 13, routine 1600 of FIG. 16, routine 1700 of FIG. 17, routine 2100, technique 3400 of FIG. 34, or the like. More generally, the instructions 3908 may cause the machine 3900 to normalize current from an ablation therapy treatment, generate graphical data for presentation on a display to provide intra-treatment information to a clinician, or interact with a database of treatment results.

The instructions 3908 transform the general, non-programmed machine 3900 into a particular machine 3900 programmed to carry out the described and illustrated functions in a specific manner. In alternative embodiments, the machine 3900 operates as a standalone device or may be coupled (e.g., networked) to other machines. In a networked deployment, the machine 3900 may operate in the capacity of a server machine or a client machine in a server-client network environment, or as a peer machine in a peer-to-peer (or distributed) network environment. The machine 3900 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 3908, sequentially or otherwise, that specify actions to be taken by the machine 3900. Further, while only a single machine 3900 is illustrated, the term “machine” shall also be taken to include a collection of machines 200 that individually or jointly execute the instructions 3908 to perform any one or more of the methodologies discussed herein.

The machine 3900 may include processors 3902, memory 3904, and I/O components 3942, which may be configured to communicate with each other such as via a bus 3944. In an example embodiment, the processors 3902 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 3906 and a processor 3910 that may execute the instructions 3908. The term “processor” is intended to include multi-core processors that may comprise two or more independent processors (sometimes referred to as “cores”) that may execute instructions contemporaneously. Although FIG. 39 shows multiple processors 3902, the machine 3900 may include a single processor with a single core, a single processor with multiple cores (e.g., a multi-core processor), multiple processors with a single core, multiple processors with multiples cores, or any combination thereof.

The memory 3904 may include a main memory 3912, a static memory 3914, and a storage unit 3916, both accessible to the processors 3902 such as via the bus 3944. The main memory 3904, the static memory 3914, and storage unit 3916 store the instructions 3908 embodying any one or more of the methodologies or functions described herein. The instructions 3908 may also reside, completely or partially, within the main memory 3912, within the static memory 3914, within machine-readable medium 3918 within the storage unit 3916, within at least one of the processors 3902 (e.g., within the processor's cache memory), or any suitable combination thereof, during execution thereof by the machine 3900.

The I/O components 3942 may include a wide variety of components to receive input, provide output, produce output, transmit information, exchange information, capture measurements, and so on. The specific I/O components 3942 that are included in a particular machine will depend on the type of machine. For example, portable machines such as mobile phones will likely include a touch input device or other such input mechanisms, while a headless server machine will likely not include such a touch input device. It will be appreciated that the I/O components 3942 may include many other components that are not shown in FIG. 39. The I/O components 3942 are grouped according to functionality merely for simplifying the following discussion and the grouping is in no way limiting. In various example embodiments, the I/O components 3942 may include output components 3928 and input components 3930. The output components 3928 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 3930 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 3942 may include biometric components 3932, motion components 3934, environmental components 3936, or position components 3938, among a wide array of other components. For example, the biometric components 3932 may include components to detect expressions (e.g., hand expressions, facial expressions, vocal expressions, body gestures, or eye tracking), measure bio-signals (e.g., blood pressure, heart rate, body temperature, perspiration, or brain waves), identify a person (e.g., voice identification, retinal identification, facial identification, fingerprint identification, or electroencephalogram-based identification), and the like. The motion components 3934 may include acceleration sensor components (e.g., accelerometer), gravitation sensor components, rotation sensor components (e.g., gyroscope), and so forth. The environmental components 3936 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 3938 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 3942 may include communication components 3940 operable to couple the machine 3900 to a network 3920 or devices 3922 via a coupling 3924 and a coupling 3926, respectively. For example, the communication components 3940 may include a network interface component or another suitable device to interface with the network 3920. In further examples, the communication components 3940 may include wired communication components, wireless communication components, cellular communication components, Near Field Communication (NFC) components, Bluetooth components (e.g., Bluetooth® Low Energy), WiFi® components, and other communication components to provide communication via other modalities. The devices 3922 may be another machine or any of a wide variety of peripheral devices (e.g., a peripheral device coupled via a USB).

Moreover, the communication components 3940 may detect identifiers or include components operable to detect identifiers. For example, the communication components 3940 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 3940, such as location via Internet Protocol (IP) geolocation, location via Wi-Fi® signal triangulation, location via detecting an NFC beacon signal that may indicate a particular location, and so forth.

The various memories (i.e., memory 3904, main memory 3912, static memory 3914, and/or memory of the processors 3902) and/or storage unit 3916 may store one or more sets of instructions and data structures (e.g., software) embodying or utilized by any one or more of the methodologies or functions described herein. These instructions (e.g., the instructions 3908), when executed by processors 3902, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 3920 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 3920 or a portion of the network 3920 may include a wireless or cellular network, and the coupling 3924 may be a Code Division Multiple Access (CDMA) connection, a Global System for Mobile communications (GSM) connection, or another type of cellular or wireless coupling. In this example, the coupling 3924 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 3908 may be transmitted or received over the network 3920 using a transmission medium via a network interface device (e.g., a network interface component included in the communication components 3940) and utilizing any one of a number of well-known transfer protocols (e.g., hypertext transfer protocol (HTTP)). Similarly, the instructions 3908 may be transmitted or received using a transmission medium via the coupling 3926 (e.g., a peer-to-peer coupling) to the devices 3922. The terms “transmission medium” and “signal medium” mean the same thing and may be used interchangeably in this disclosure. The terms “transmission medium” and “signal medium” shall be taken to include any intangible medium that is capable of storing, encoding, or carrying the instructions 3908 for execution by the machine 3900, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment,” “an embodiment,” “one example,” “an example, or “embodiments” and “examples” in the plural do not necessarily refer to the same embodiment or require plural embodiments, although it may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

The following examples pertain to embodiments of the disclosure, of which, numerous permutations and configurations will be apparent.

A first example is an ablation therapy device, comprising: a generator, the generator to operatively couple to a plurality of electrodes; control circuitry coupled to the generator; and a memory device storing instructions that when executed by the control circuitry cause the control circuitry to: identify a one of a plurality of predefined intensity levels; generate a treatment program based on the one of the plurality of predefined intensity levels, the treatment program comprising indications of a series of electrical pulses to be applied through the electrodes to a target tissue; and send a control signal to the generator to cause the generator to generate the series of electrical pulses.

A second example is the ablation therapy device of the first example, the memory device further storing instructions, which when executed cause the control circuitry to receive an indication of the one of the plurality of predefined intensity levels.

A third example is the ablation therapy device of the first example, the target tissue comprising a tumor, the memory device further storing instructions, which when executed cause the control circuitry to: receive an indication of an immunoscore of the tumor; and determine the one of the plurality of predefined intensity levels based on the immunoscore.

A fourth example is the ablation therapy device of the third example, the memory device further storing instructions, which when executed cause the control circuitry to: determine an ablation modality of a plurality of ablation modalities based on the immunoscore; and generate the treatment program based on ablation modality of the plurality of ablation modalities and the one of the plurality of predefined intensity levels.

A fifth example is the ablation therapy device of the fourth example, wherein the plurality of ablation modalities comprising irreversible electroporation (IRE), high-frequency (IRE) (HFIRE), non-thermal ablation, reversible electroporation, or pulse field ablation.

A sixth example is the ablation therapy device of the fourth example, the memory device further storing instructions, which when executed cause the control circuitry to: determine a recommended complimentary therapy to be applied based on the immunoscore; and generate the treatment program based on the recommended complementary therapy and the one of the plurality of predefined intensity levels.

A seventh example is the ablation therapy device of the sixth example, wherein the adjunct therapy includes at least one of: immunotherapy, chemotherapy, radiation, vaccination, and surgical intervention.

An eight example is the ablation therapy device of the first example, comprising a sensor, the sensor arranged to measure a current produced responsive to application of the electrical pulses of the series of electrical pulses to the target tissue, the memory device further storing instructions, which when executed cause the control circuitry to: receive from the sensor, an indication of the current; and determine whether the one of the plurality of predefined intensity levels is met or exceeded based on the indication of the current.

A ninth example is the ablation therapy device of the eighth example, the memory device further storing instructions, which when executed cause the control circuitry to send a control signal to the generator to cause the generator to pause generation of the series of electrical pulses based on a determination that the one of the plurality of predefined intensity levels is met or exceeded.

A tenth example is the ablation therapy device of the eighth example, comprising a display unit coupled to the control circuitry, the memory device further storing instructions, which when executed cause the control circuitry to: generate a graphical information element comprising an indication that the one of the plurality of predefined intensity levels is met or exceeded based on a determination that the one of the plurality of predefined intensity levels is met or exceeded; and send the graphical information element to the display unit to cause the display unit to display the indication that the one of the plurality of predefined intensity levels is met or exceeded.

An eleventh example is the ablation therapy device of the tenth example, the memory device further storing instructions, which when executed cause the control circuitry to: receive, responsive to displaying the graphical information element, an indication to continue application of electrical pulses to the target tissue; identify another one of the plurality of predefined intensity levels; generate a secondary treatment program based on the other one of the plurality of predefined intensity levels, the secondary treatment program comprising indications of a secondary series of electrical pulses to be applied through the electrodes to the target tissue; and send a second control signal to the generator to cause the generator to generate the secondary series of electrical pulses.

A twelfth example is the ablation therapy device of the eighth example, the memory device further storing instructions, which when executed cause the control circuitry to: normalize the current for a first electrical pulse of the series of electrical pulses; normalize the current for a second electrical pulse of the series of electrical pulses; determine whether a difference between the normalized current for the first electrical pulse and the normalized current for the second electrical pulse is greater than or equal to a threshold value; and determine that the one of the plurality of predefined intensity levels is met or exceeded based on a determination that the difference between the normalized current for the first electrical pulse and the normalized current for the second electrical pulse is greater than or equal to the threshold value.

A thirteenth example is the ablation therapy device of the first example, the treatment program comprising a plurality of series of electrical pulses to be applied through the electrodes to a target tissue.

A fourteenth example is the ablation therapy device of the first example, the series of electrical pulses are sufficient to reversibly electroporate cells within the target tissue, irreversibly electroporate cells within the target tissue, thermally ablate cells within the target tissue, and/or result in electrolysis of cells within the target tissue.

A fifteenth example is the ablation therapy device of the first example, the plurality of predefined intensity levels comprising at least a first intensity level, a second intensity level greater than the first intensity level, and a third intensity level greater than the second intensity level.

A sixteenth example is the ablation therapy device of the fifteenth example, wherein the first intensity level is associated with a first tumor type, the second intensity level is associated with a second tumor type different than the first tumor type, and the third intensity level is associated with a third tumor type different than the first tumor type and the second tumor type.

A seventeenth example is the ablation therapy device of the sixteenth example, wherein the first tumor type is a hot tumor, the second tumor type is an altered tumor, and the third tumor type is a cold tumor.

An eighteenth example is the ablation therapy device of the seventeenth example, wherein the series of electrical pulses are sufficient to change the tumor type from a cold tumor to either an immunosuppressive tumor or a hot tumor, or from an immunosuppressive tumor to a hot tumor.

A nineteenth example is the ablation therapy device of the eighteenth example, wherein the memory device further storing instructions, which when executed cause the control circuitry to: determine a complementary therapy to be applied to the changed tumor; and generate the treatment program based on the complementary therapy and the series of electrical pulses.

A twentieth example is the ablation therapy device of the nineteenth example, wherein the complementary therapy includes at least one of:
immunotherapy, chemotherapy, radiation, vaccination, and surgical intervention.

A twenty-first example is the ablation therapy device of the first example, the memory device storing a machine learning (ML) model and further storing instructions, which when executed cause the control circuitry to execute the ML model to generate the treatment program.

A twenty-second example is a method, comprising: identifying, at a computing device, a one of a plurality of predefined intensity levels; generate, at the computing device, a graphical information element comprising an indication of the one of the plurality of predefined intensity levels; send the graphical information element to a display unit coupled to the computing device to cause the display unit to display the indication; receive, at the computing device, responsive to displaying the graphical information element, an indication to select the one of the plurality of predefined intensity levels; generate, at the computing device, a treatment program based on the one of the plurality of predefined intensity levels, the treatment program comprising indications of a series of electrical pulses to be applied through the electrodes to a target tissue; and sending, by the computing device, a control signal to the generator to cause the generator to generate the series of electrical pulses.

A twenty-third example is the method of method of the twenty-second example, the target tissue comprising a tumor, the method comprising: receiving, at the computing device, an indication of an immunoscore of the tumor; and determining, by the computing device, the one of the plurality of predefined intensity levels based on the immunoscore.

A twenty-fourth example is the method of the twenty-third example, comprising: determining, at the computing device, an ablation modality of a plurality of ablation modalities based on the immunoscore; and generating, by the computing device, the treatment program based on ablation modality of the plurality of ablation modalities and the one of the plurality of predefined intensity levels.

A twenty-fifth example is the method of the twenty-fourth example, wherein the plurality of ablation modalities comprising irreversible electroporation (IRE) or high-frequency (IRE) (HFIRE).

A twenty-sixth example is the method of the twenty-fourth example, comprising: determining, at the computing device, a complementary therapy to be applied based on the immunoscore; and generating, by the computing device, the treatment program based on the complementary therapy and the one of the plurality of predefined intensity levels.

A twenty-seventh example is the method of the twenty-sixth example, wherein the complementary therapy includes at least one of: immunotherapy, chemotherapy, radiation, vaccination, and surgical intervention.

A twenty-eight example is the method of the twenty-second example, comprising: receiving, at the computing device, from a sensor, an indication of a current produced responsive to application of the electrical pulses of the series of electrical pulses to the target tissue, the sensor arranged to measure the current; and determining, at the computing device, whether the one of the plurality of predefined intensity levels is met or exceeded based on the indication of the current.

A twenty-ninth example is the method of the twenty-eighth example, comprising sending a control signal to the generator to cause the generator to pause generation of the series of electrical pulses based on a determination that the one of the plurality of predefined intensity levels is met or exceeded.

A thirtieth example is the method of the twenty-eighth example, comprising: generating, by the computing device, a second graphical information element comprising an indication that the one of the plurality of predefined intensity levels is met or exceeded based on a determination that the one of the plurality of predefined intensity levels is met or exceeded; and sending the second graphical information element to the display unit to cause the display unit to display the indication that the one of the plurality of predefined intensity levels is met or exceeded.

A thirty-first example is the method of the thirtieth example, comprising: receiving, at the computing device, responsive to displaying the second graphical information element, an indication to continue application of electrical pulses to the target tissue; identifying, at the computing device, another one of the plurality of predefined intensity levels; generating, by the computing device, a secondary treatment program based on the other one of the plurality of predefined intensity levels, the secondary treatment program comprising indications of a secondary series of electrical pulses to be applied through the electrodes to the target tissue; and sending a second control signal to the generator to cause the generator to generate the secondary series of electrical pulses.

A thirty-second example is the method of the twenty-eighth example, comprising: normalizing, by the computing device, the current for a first electrical pulse of the series of electrical pulses; normalizing, by the computing device, the current for a second electrical pulse of the series of electrical pulses; determining, at the computing device, whether a difference between the normalized current for the first electrical pulse and the normalized current for the second electrical pulse is greater than or equal to a threshold value; and determining, at the computing device, that the one of the plurality of predefined intensity levels is met or exceeded based on a determination that the difference between the normalized current for the first electrical pulse and the normalized current for the second electrical pulse is greater than or equal to the threshold value.

A thirty-third example is the method of the twenty-second example, the treatment program comprising a plurality of series of electrical pulses to be applied through the electrodes to a target tissue.

A thirty-fourth example is the method of the twenty-second example, the series of electrical pulses are sufficient to reversibly electroporate cells within the target tissue, irreversibly electroporate cells within the target tissue, thermally ablate cells within the target tissue, and/or result in electrolysis of cells within the target tissue.

A thirty-fifth example is the method of the twenty-second example, the plurality of predefined intensity levels comprising at least a first intensity level, a second intensity level greater than the first intensity level, and a third intensity level greater than the second intensity level.

A thirty-sixth example is the method of the thirty-fifth example, wherein the first intensity level is associated with a first tumor type, the second intensity level is associated with a second tumor type different than the first tumor type, and the third intensity level is associated with a third tumor type different than the first tumor type and the second tumor type.

A thirty-seventh example is the method of the thirty-sixth example, wherein the first tumor type is a hot tumor, the second tumor type is an altered tumor, and the third tumor type is a cold tumor.

A thirty-eighth example is the method of the thirty-seventh example, wherein the series of electrical pulses are sufficient to change the tumor type from a cold tumor to either an immunosuppressive tumor or a hot tumor, or from an immunosuppressive tumor to a hot tumor.

A thirty-ninth example is the method of the thirty-eighth example, comprising: determining, by the computing device, a complementary therapy to be applied to the changed tumor; and generating, at the computing device, the treatment program based on the complementary therapy and the series of electrical pulses.

A fortieth example is the method of the thirty-ninth example, wherein the complementary therapy includes at least one of: immunotherapy, chemotherapy, radiation, vaccination, and surgical intervention.

A forty-first example is the method of the twenty-second example, comprising execute a machine learning (ML) model to generate the treatment program.

A forty-second example is at least one non-transitory computer-readable storage medium comprising instructions that when executed by a computing device, cause the computing device to perform the method of any one of the twenty-second to the forty-first examples.

What is claimed is:

1. An ablation therapy device, comprising:
a generator, the generator to operatively couple to a plurality of electrodes;
control circuitry coupled to the generator; and
a memory device storing instructions that when executed by the control circuitry cause the control circuitry to:
identify a one of a plurality of predefined intensity levels;
generate a treatment program based on the one of the plurality of predefined intensity levels, the treatment program comprising indications of a series of electrical pulses to be applied through the electrodes to a target tissue; and
send a control signal to the generator to cause the generator to generate the series of electrical pulses.

2. The ablation therapy device of claim 1, the memory device further storing instructions, which when executed cause the control circuitry to receive an indication of the one of the plurality of predefined intensity levels.

3. The ablation therapy device of claim 1, the target tissue comprising a tumor, the memory device further storing instructions, which when executed cause the control circuitry to:
receive an indication of an immunoscore of the tumor; and
determine the one of the plurality of predefined intensity levels based on the immunoscore.

4. The ablation therapy device of claim 3, the memory device further storing instructions, which when executed cause the control circuitry to:
determine an ablation modality of a plurality of ablation modalities based on the immunoscore; and
generate the treatment program based on ablation modality of the plurality of ablation modalities and the one of the plurality of predefined intensity levels.

5. The ablation therapy device of claim 4, wherein the plurality of ablation modalities comprising irreversible electroporation (IRE), high-frequency (IRE) (HFIRE), non-thermal ablation, reversible electroporation, or pulsed field ablation.

6. The ablation therapy device of claim 4, the memory device further storing instructions, which when executed cause the control circuitry to:
determine a recommended complementary therapy to be applied based on the immunoscore; and
generate the treatment program based on the recommended complementary therapy and the one of the plurality of predefined intensity levels.

7. The ablation therapy device of claim 6, wherein the recommended complementary therapy includes at least one of: immunotherapy, chemotherapy, radiation, vaccination, and surgical intervention.

8. The ablation therapy device of claim 1, comprising a sensor, the sensor arranged to measure a characteristic produced responsive to application of the electrical pulses of the series of electrical pulses to be applied to through the electrodes to the target tissue, the memory device further storing instructions, which when executed cause the control circuitry to:
receive from the sensor, an indication of the characteristic; and
determine whether the one of the plurality of predefined intensity levels is met or exceeded based on the indication of the characteristic.

9. The ablation therapy device of claim 8, the memory device further storing instructions, which when executed cause the control circuitry to send a control signal to the generator to cause the generator to pause generation of the series of electrical pulses based on a determination that the one of the plurality of predefined intensity levels is met or exceeded.

10. The ablation therapy device of claim 8, comprising a display unit coupled to the control circuitry, the memory device further storing instructions, which when executed cause the control circuitry to:
generate a graphical information element comprising an indication that the one of the plurality of predefined intensity levels is met or exceeded based on a determination that the one of the plurality of predefined intensity levels is met or exceeded; and
send the graphical information element to the display unit to cause the display unit to display the indication that the one of the plurality of predefined intensity levels is met or exceeded.

11. The ablation therapy device of claim 10, the memory device further storing instructions, which when executed cause the control circuitry to:
receive, responsive to displaying the graphical information element, an indication to continue application of electrical pulses to the target tissue;
identify another one of the plurality of predefined intensity levels;
generate a secondary treatment program based on the other one of the plurality of predefined intensity levels, the secondary treatment program comprising indications of a secondary series of electrical pulses to be applied through the electrodes to the target tissue; and
send a second control signal to the generator to cause the generator to generate the secondary series of electrical pulses.

12. The ablation therapy device of claim 8, the memory device further storing instructions, which when executed cause the control circuitry to:
normalize the current for a first electrical pulse of the series of electrical pulses;
normalize the current for a second electrical pulse of the series of electrical pulses;
determine whether a difference between the normalized current for the first electrical pulse and the normalized current for the second electrical pulse is greater than or equal to a threshold value; and
determine that the one of the plurality of predefined intensity levels is met or exceeded based on a determination that the difference between the normalized current for the first electrical pulse and the normalized current for the second electrical pulse is greater than or equal to the threshold value.

13. The ablation therapy device of claim 1, the series of electrical pulses are sufficient to reversibly electroporate cells within the target tissue, irreversibly electroporate cells within the target tissue, thermally ablate cells within the target tissue, non-thermally ablate cells within the target tissue, and/or result in electrolysis of cells within the target tissue.

14. The ablation therapy device of claim 1, the plurality of predefined intensity levels comprising at least a first intensity level, a second intensity level greater than the first intensity level, and a third intensity level greater than the second intensity level.

15. The ablation therapy device of claim 14, wherein the first intensity level is associated with a first tumor type, the second intensity level is associated with a second tumor type different than the first tumor type, and the third intensity level is associated with a third tumor type different than the first tumor type and the second tumor type, wherein the first tumor type is a hot tumor, the second tumor type is an altered tumor, and the third tumor type is a cold tumor.

16. The ablation therapy device of claim 15, wherein the series of electrical pulses are sufficient to change the tumor type from a cold tumor to either an immunosuppressive tumor or a hot tumor, or from an immunosuppressive tumor to a hot tumor.

17. The ablation therapy device of claim 16, wherein the memory device further storing instructions, which when executed cause the control circuitry to:
determine a complementary therapy to be applied to the changed tumor; and
generate the treatment program based on the complementary therapy and the series of electrical pulses.

18. The ablation therapy device of claim 17, wherein the complementary therapy includes at least one of: immunotherapy, chemotherapy, radiation, vaccination, and surgical intervention.

19. An ablation therapy device, comprising:
a generator, the generator to operatively couple to a plurality of electrodes;
control circuitry coupled to the generator; and
a memory device storing instructions that when executed by the control circuitry cause the control circuitry to:
receive an indication of an immunoscore of a target tissue;
determine an ablation modality of a plurality of ablation modalities based on the immunoscore;
identify a one of a plurality of predefined intensity levels based on the immunoscore;
generate a treatment program based on the one of the plurality of predefined intensity levels and the ablation modality of the plurality of ablation modalities, the treatment program comprising indications of a series of electrical pulses to be applied through the electrodes to the target tissue; and
send a control signal to the generator to cause the generator to generate the series of electrical pulses.

20. An ablation therapy device, comprising:
a generator, the generator to operatively couple to a plurality of electrodes;
control circuitry coupled to the generator;
a sensor, the sensor arranged to measure a current produced responsive to application of electrical pulses of a series of electrical pulses to a target tissue;
a memory device storing instructions that when executed by the control circuitry cause the control circuitry to:
identify a one of a plurality of predefined intensity levels;
generate a treatment program based on the one of the plurality of predefined intensity levels, the treatment program comprising indications of the series of electrical pulses to be applied through the electrodes to the target tissue;
send a control signal to the generator to cause the generator to generate the series of electrical pulses;
receive from the sensor an indication of the current produced responsive to application of the electrical pulses of the series of electrical pulses to the target tissue; and
determine whether the one of the plurality of predefined intensity levels is met or exceeded based on the indication of the current received from the sensor.
